(19) United States
(12) Patent Application Publication
(10) Pub. No.: US 2026/0240452 A1
(43) Pub. Date: Aug. 20, 2026

(54) SYSTEMS AND METHODS FOR MAPPING OF GASTROINTESTINAL TRACT

(71) Applicants: Massachusetts Institute of Technology, Cambridge, MA (US); THE BRIGHAM AND WOMEN'S HOSPITAL, INC., Boston, MA (US)

(72) Inventors: Zixun JIA, Cambridge, MA (US); Carlo Giovanni TRAVERSO, Cambridge, MA (US)

(21) Appl. No.: 19/152,715

(22) PCT Filed: Jan. 29, 2024
(86) PCT No.: PCT/US2024/013380

§ 371 Date: Jul. 31, 2025

Related U.S. Application Data
(60) Provisional application No. 63/442,732, filed on Feb. 1, 2023.

(51) Int. Cl. A61B 5/107; A61B 5/00; A61M 25/00; A61M 25/01; A61M 25/06

(52) U.S. Cl. CPC A61B 5/1076; A61B 5/42; A61B 5/6852; A61B 2562/0247; A61B 2562/04; A61B 2562/12; A61B 2562/164; A61B 2562/166; A61M 25/0009; A61M 25/0158; A61M 2025/0681

(57) ABSTRACT

A catheter system for obtaining a pressure profile of a gastrointestinal (GI) tract includes a catheter configured to be inserted endoluminally into the GI tract. The catheter includes an outer sheath defining a first lumen and sensing unit enclosed within the first lumen. The sensing unit includes a plurality of pressure sensors spaced apart along the flexible printed circuit board that is supported on a support structure. The support structure is made of a shape memory material that is configured to transition from a first configuration to a second configuration following insertion of the catheter into the GI tract. The second configuration includes a first region shaped to conform to a first portion of the GI tract and a second region shaped to conform to a second portion of the GI tract that is distinctly shaped from the first portion of the GI tract.

### CROSS-REFERENCE TO RELATED APPLICATIONS

The present application is based on and claims the benefit of U.S. Provisional Patent Application Ser. No. 63/442,732, filed Feb. 1, 2023, which is incorporated herein by reference in its entirety.

### STATEMENT REGARDING FEDERALLY SPONSORED RESEARCH

N/A

### BACKGROUND

The present disclosure relates generally to pressure sensors and pressure sensing systems for use in gastrointestinal (GI) mapping, and more specifically, for the diagnosis and monitoring of GI motility disorders. GI dysmotility can affect any part of the alimentary tract and may manifest in or contribute to digestive conditions including gastroesophageal reflux disease, gastroparesis, intestinal pseudo-obstruction, irritable bowel syndrome, chronic constipation, and fecal incontinence. Not only do these symptoms rank among the most common patient presentations, but they are also associated with significant morbidity, including malnutrition, feeding tube dependency, need for invasive surgery, frequent hospitalizations, and death.

The current evaluation of patients with these symptoms involves multiple diagnostic elements, including, for example, enterography, scintigraphy, and manometry. In particular, the evaluation of tone and contractile patterns of the GI tract is an essential aspect in the diagnosis of GI motility disorders, with manometry playing one of the most important roles. Manometry involves placing a manometer, which is a catheter-like device containing a series of pressure transducers located in the catheter, endoluminally into the GI tract of a patient to measure real-time pressure changes along the length of the device. The pressure changes that are measured result from the peristaltic contractions of the patient's GI tract and can be used to identify regions with impaired motility and other types of GI motility disorders.

Several forms of tools for GI mapping have been developed to evaluate the specific segments of the alimentary tract, including esophageal, antroduodenal, colonic, and anorectal portions of the GI tract. For example, wireless motility capsules, impedance planimetry, enterography, high resolution manometry, and scintigraphy can be used to map various portions of a GI tract. However, the existing diagnosis intervention and tools have limitations in terms of sensing ability. In particular, impedance planimetry, high resolution manometry, and anorectal manometry all use catheter-like sensing probes and, thus, are only suitable for tubular organs like the esophagus and rectum. Wireless motility capsules can measure the pH, pressure, and temperature inside the GI tract and transfer the data back to a PC, and can only provide that data with respect to transit time in the GI tract. Enterography and scintigraphy can provide 2D videos/pictures of the GI tract but cannot provide spatial pressure distribution. Also, in scintigraphy, patients are exposed to radiation.

Further, current systems for assessing and evaluating GI motility disorders, particularly those for mapping and characterizing GI motility in an in vivo manner, generally provide only zero-, one-, or two-dimensional mapping of select portions of the GI tract. Moreover, current systems are typically only suitable for assessing GI motility within substantially tubular portions of the GI tract, for example, the esophagus or rectum, and not for non-tubular regions such as the stomach. Further, current systems also suffer from high cost, complexity, and bulkiness that limit their use in less developed regions or non-hospital settings, which can limit a physician's ability to assess and diagnose GI dysmotility conditions in resource-constrained settings.

In view of the above, a need exists for an improved GI mapping system that can provide for high-resolution and high density pressure measurements to provide three-dimensional mapping of an entire gastrointestinal tract, or portions thereof (e.g., the esophagus, stomach, and rectum), while also being simple and cheap to manufacture from easily accessible and economical materials. Additionally, it is desirable to provide a GI manometer system that can either be easily disinfected or disposable, and which can measure pressure along a substantial or entire length of a patient's GI tract. The discussion above is merely provided for general background information and is not intended to unduly limit the scope of the claimed subject matter.

### SUMMARY

Aspects of the present disclosure, as generally disclosed herein, can provide for an economical and easy to manufacture GI mapping or other monitoring system that can provide three-dimensional pressure measurements of a GI tract of a patient (e.g., an entirety of a GI tract or select portions thereof). Such a system can include a sensor configured as a pressure-sensing catheter (e.g., a sensing probe) having a flexible printed circuit board (e.g., a sensing unit) that supports a plurality of pressure sensors. The flexible printed circuit board can be supported on a base structure that is made of a shape-memory material (e.g., an alloy, polymer, or other material). Correspondingly, the catheter can have a first configuration (e.g., a linear configuration) at a first stimulus condition (e.g., a first temperature condition) for insertion into a GI tract. Following insertion into the GI tract, the catheter, via the base structure, can transition to a second configuration (e.g., a nonlinear configuration) at a second stimulus condition (e.g., a second temperature condition). The second configuration of the catheter can be configured to conform to the shape of a specific organ or section of the GI tract, thereby orienting the pressure sensors to be compressed by the GI tract. Accordingly, the sensor system can be configured to conform to the specific anatomic environment, including tubular structures like the esophagus, as well as non-tubular structures like the stomach.

In accordance with one aspect of the present disclosure, a catheter system for obtaining a pressure profile of a gastrointestinal (GI) tract can include a catheter configured to be inserted endoluminally into the GI tract. The catheter can include an outer sheath defining a first lumen and sensing unit enclosed within the first lumen The sensing unit can include a support structure, a flexible printed circuit board supported on the support structure, and a plurality of pressure sensors spaced apart along the flexible printed circuit board. The support structure can be made of a shape memory material that can be configured to transition from a first configuration to a second configuration. The second configuration can include a first region shaped to conform to a first portion of the GI tract and a second region shaped to conform to a second portion of the GI tract. The second portion can be distinctly shaped from the first portion of the GI tract. Following insertion of the catheter into the GI tract, the support structure can cause the sensing unit to transition from the first configuration to the second configuration to position the plurality of pressure sensors to acquire pressure readings in three dimensions from the first portion of the GI tract and the second portion of the GI tract.

In accordance with another aspect of the present disclosure, a pressure sensor can include a flexible printed circuit board having at least one u-shaped section defining a first arm and a second arm. A compression member can be disposed between the first arm and the second arm so that a first end of the compression member is coupled to the first arm and a second end of the compression member is coupled to the second arm. The compression member can be configured to receive a compression of the first and second arm to induce a change in an electrical resistance of the compression member correlated to a measure of the compression.

In accordance with yet another aspect of the present disclosure, a method of manufacturing a catheter system for obtaining a pressure profile of a gastrointestinal (GI) tract can include securing a first end of a compression member to a first arm of a flexible printed circuit board. A second arm of the flexible printed circuit board can be folded relative to the first arm so that first and second arms form a u-shape. A second end of the compression member can be secured to the second arm so that the compression member extends between the first and second arms. The compression member can be configured to be resiliently compressed by the first and second arms to induce a change in an electrical resistance of the compression member.

This Summary and the Abstract are provided to introduce a selection of concepts in a simplified form that are further described below in the Detailed Description. This Summary is not intended to identify key features or essential features of the claimed subject matter, nor are they intended to be used as an aid in determining the scope of the claimed subject matter.

### BRIEF DESCRIPTION OF THE DRAWINGS

The following drawings are provided to help illustrate various features of non-limiting examples of the disclosure and are not intended to limit the scope of the disclosure or to exclude alternative implementations.

FIG. 1 is a perspective view of an exemplary catheter system having a pressure sensing catheter according to aspects of the present disclosure.

FIG. 2 is an exploded view of the catheter of FIG. 1.

FIG. 3 is a schematic view of various regions of the catheter of FIG. 1 inserted into and conforming with a GI tract.

FIG. 4 is a perspective view of a pressure sensor of a sensing unit of the catheter of FIG. 1.

FIG. 5 is a schematic view of the pressure sensor of FIG. 4.

FIG. 6 depicts images of a foam cell structure for a compression member that can be used in a pressure sensor.

FIG. 7 is plot comparing the electric conductivity of various foams for a compression member that can be used in a pressure sensor.

FIG. 8 is a plot of stress-strain curves of the foams of FIG. 7.

FIG. 9 is a plot comparing the Young's Modulus of the foams of FIG. 7.

FIG. 10 is a plot of the gauge factor of 16-1231 foam under various stroke speeds.

FIG. 11 is a plot comparing changes in sensor height for various applied pressures and material thicknesses.

FIG. 12 is another plot comparing changes in sensor height for various applied pressures and material thicknesses.

FIG. 13 is a plot showing the mechanical deformation-force relationship of a single sensor cell.

FIG. 14 is a schematic view of a channel resistance scanning board for a measurement device of a catheter system.

FIG. 15 is a schematic view of a single channel switch gate structure for a measurement device of a catheter system.

FIG. 16 is a schematic view of a column-rows scanning structure for a measurement device of a catheter system.

FIG. 17 is a plot showing an axial stiffness of a bolus structure of a catheter.

FIG. 18A is perspective view of a curved bolus portion of a catheter formed by a plurality of coils.

FIG. 18B is perspective view of a curved bolus portion of a FIG. 18A with arrows indicating axial stiffness.

FIG. 18C is perspective view of a curved bolus portion of a FIG. 18A with arrows indicating radial stiffness.

FIG. 19 is a plot showing model predicted radial stiffness of a bolus portion of a catheter.

FIG. 20 is a plot showing experimental data of radial stiffness of a bolus portion of a catheter.

FIG. 21 is a plot showing frictional forces on a catheter during retrieval from a GI tract.

FIG. 22 is a plot showing frictional forces on a catheter during insertion into a GI tract.

FIG. 23 is a plot showing temperature of a support structure as a function of time in an overtube.

FIG. 24 is a schematic view a fabrication process for a bolus supporting structure fabrication and integration of sensors/supporting structure.

FIG. 25 is an image showing an ex vivo test of a catheter system and the resultant pressure maps, with arrows indicating areas of applied pressure.

FIG. 26 is a plot showing recorded pressure measurements in an esophagus stimulated by a bolus balloon.

FIG. 27 is a plot showing recorded pressure measurements in an esophagus stimulated by a dilation catheter.

FIG. 28 is a plot showing recorded pressure measurements in a rectum stimulated by a bolus balloon.

FIG. 29 is a schematic view illustrating an in vivo test in a swine model, wherein external pressure applied to an abdomen is mapped by a catheter system according to the disclosure.

FIG. 30 is a schematic view illustrating an in vivo test in a swine model, wherein an internal force applied by inflating and deflating a stomach with an endoscope is mapped by a catheter system according to the disclosure.

FIGS. 31A-C is a schematic view of a deployment and retrieval process for a catheter system according to the disclosure.

FIG. 32 is a schematic view illustrating a setup for a swine model motility mapping test.

FIG. 33 is an image of a catheter according to the disclosure being inserted into an esophagus of a swine model.

FIG. 34 is an image of a catheter according to the disclosure being inserted into a rectum of a swine model.

FIG. 35 is an image of a catheter according to the disclosure being inserted into a stomach of a swine model.

FIG. 36 is another image of the catheter of FIG. 33.

FIG. 37 is an image of a mixture of THE, MWCNT, and SEBS polymer cast on a glass sheet, shown in both an uncured state (left) and cured state (right).

FIG. 38 is plot showing a resistance-force curve for a SEBS film.

FIG. 39 is schematic view of a fabrication process for a SEBS and MWCNT porous foam.

FIG. 40 is an image of a SEBS and MWCNT porous foam made using the process of FIG. 39.

FIG. 41 is another image showing a cellular structure of the SEBS and MWCNT porous foam of FIG. 40.

FIG. 42 is a plot of a resistance-pressure curve of a SEBS and MWCNT porous foam.

FIG. 43 is an image of Eco-flex and MWCNT foam made using the process of FIG. 39.

FIG. 44 is a plot showing the conductivity of the Eco-flex and MWCNT foam of FIG. 43 as a function of the percent weight of MWCNT.

FIG. 45 is a plot showing stress-strain curves for various Eco-flex and MWCNT foam samples with different percent weights of MWCNT.

FIG. 46 is plot showing the leftover carbon residue that is left after curing for various Eco-flex and MWCNT foam samples with different percent weights of MWCNT.

FIG. 47 is a plot showing the degradation rate for various Eco-flex and MWCNT foam samples with different percent weights of MWCNT.

FIG. 48 is plot showing a thermogravimetric analysis for various Eco-flex and MWCNT foam samples with different percent weights of MWCNT.

FIG. 49 is an image of a foam adhesion test for an Eco-flex and MWCNT foam.

FIG. 50 is an image of a foam adhesion test for 16-1231 foam.

FIG. 51 is a schematic view of a single pressure sensor according to the disclosure.

FIG. 52 is a schematic view illustrating a twin spring model of the pressure sensor of FIG. 51.

FIG. 53 is an image showing the compression of the pressure sensor of FIG. 51.

FIG. 54 is an image showing the cellular structure of four types of foams.

FIG. 55 is a plot showing the porosity of each of the four types of foams of FIG. 54.

FIG. 56 is a plot showing the conductivity of each of the four types of foams of FIG. 54.

FIG. 57 is a plot showing the mechano-electrical performance of the pressure sensor of FIG. 51.

FIG. 58 is plot showing the change in resistance over time for various load cycles of the pressure sensor of FIG. 51.

FIG. 59 is schematic view of a row part of a scanning board row part according to the disclosure.

FIG. 60 is a schematic view of a column part of the scanning board of FIG. 59.

FIG. 61 is a schematic view of a printed circuit board of the scanning board of FIG. 59.

FIG. 62 is a schematic view of a resistance reference board according to the disclosure.

FIG. 63 is a schematic view of a printed circuit board of the resistance reference board of FIG. 62.

FIG. 64 is a schematic view of the resistor array according to the disclosure.

FIG. 65 is an image of a front of the resistor array of FIG. 64.

FIG. 66 is an image of a back of the resistor array of FIG. 64.

FIG. 67 is an image of voltage measurements taken with the scanning board of FIG. 59 with the resistor array of FIG. 64 being connected to rows 1-5 and columns 1-8.

FIG. 68 is an image of resistance measurements taken with the scanning board of FIG. 59 with the resistor array of FIG. 64 being connected to rows 1-5 and columns 1-8.

FIG. 69 is an image of voltage measurements taken with the scanning board of FIG. 59 with the resistor array of FIG. 64 being connected to rows 10-14 and columns 9-16.

FIG. 70 is an image of resistance measurements taken with the scanning board of FIG. 59 with the resistor array of FIG. 64 being connected to rows 10-14 and columns 9-16.

FIG. 71 is a plot showing a resistance calculation curve.

FIG. 72 is an image of a compression test setup for a catheter according to the disclosure.

FIG. 73 is an image of a compression pattern used in the compression test setup of FIG. 72.

FIG. 74 is plot showing a pressure map obtained with a catheter according to the disclosure, during a compression test using the compression pattern of FIG. 73.

FIG. 75 in an image showing a tubular sensor band, i.e., catheter, calibration setup according to the disclosure.

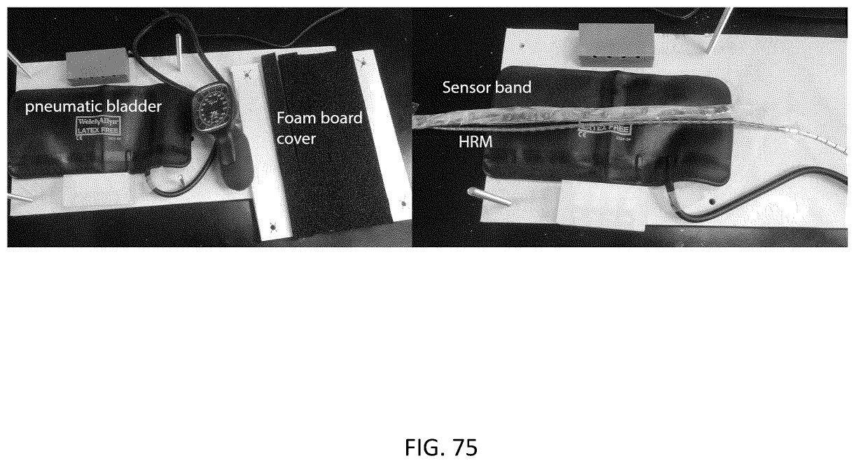

FIG. 76 is an image comparing the tubular sensor band of FIG. 75 with a conventional HRM device.

FIG. 77 is a plot showing calibration curves for multiple sensors of the tubular sensor band of FIG. 75.

FIG. 78 is plot showing a measured pressure as a function of time and location for the tubular sensor band of FIG. 75.

FIG. 79 is plot showing a measured pressure as a function of time and location for the HRM device of FIG. 76.

FIG. 80 is an image of a stomach of swine model according to the disclosure.

FIG. 81 is a schematic view of an axial stiffness model of a bolus portion of a catheter according to the disclosure.

FIG. 82 is a schematic view of a radial stiffness model of a bolus portion of a catheter according to the disclosure.

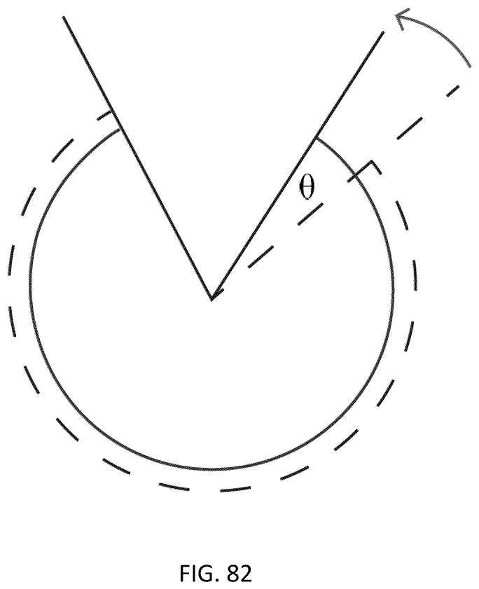

FIG. 83 is a schematic of view a friction model of a bolus portion of a catheter according to the disclosure.

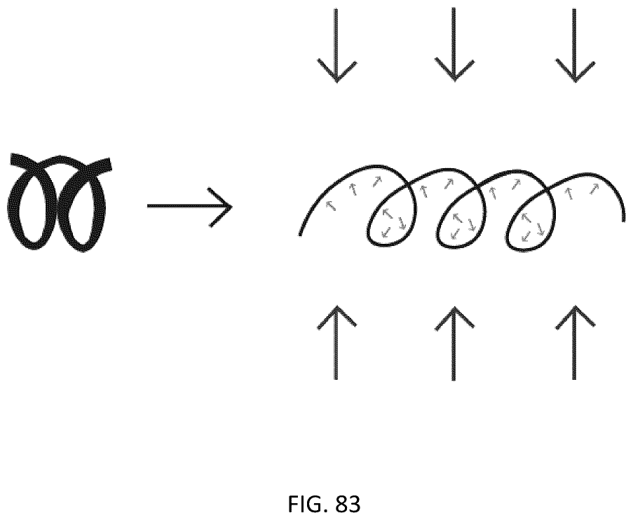

FIG. 84 is a schematic view showing a cross section of a single pressure sensor according to the disclosure.

FIG. 85 is a schematic view of a heat transfer model for the pressure sensor of FIG. 84.

FIG. 86 is a schematic view of a relaxed and final open angle of a bolus portion.

FIG. 87 is a schematic of an open angle of a bolus portion at 100 g/m for various thicknesses of nitinol support structures.

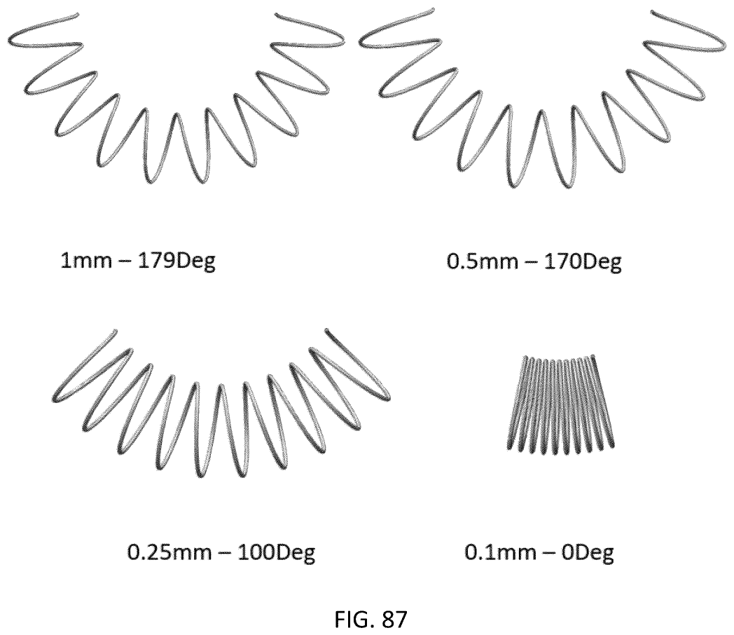

FIG. 88 is perspective view of a heat treatment fixture for a nitinol support structure for use in making a bolus portion of catheter according to the disclosure.

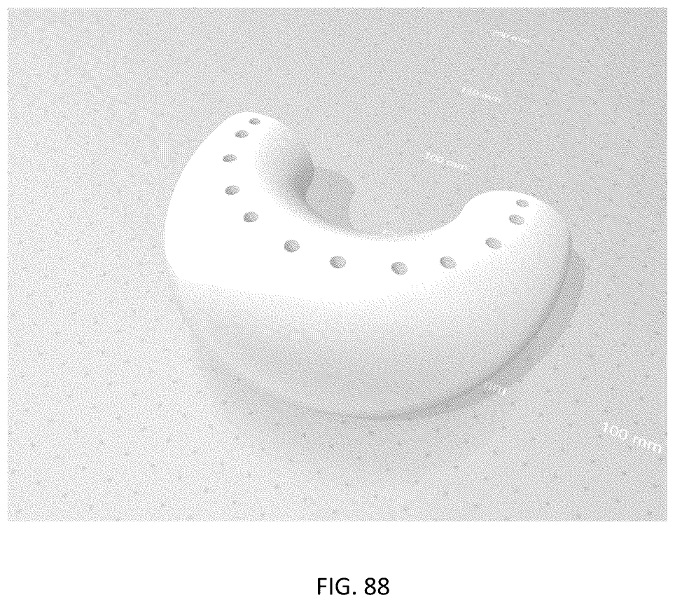

FIG. 89 is an image of a nitinol support structure being heat treated on the heat treatment fixture of FIG. 88.

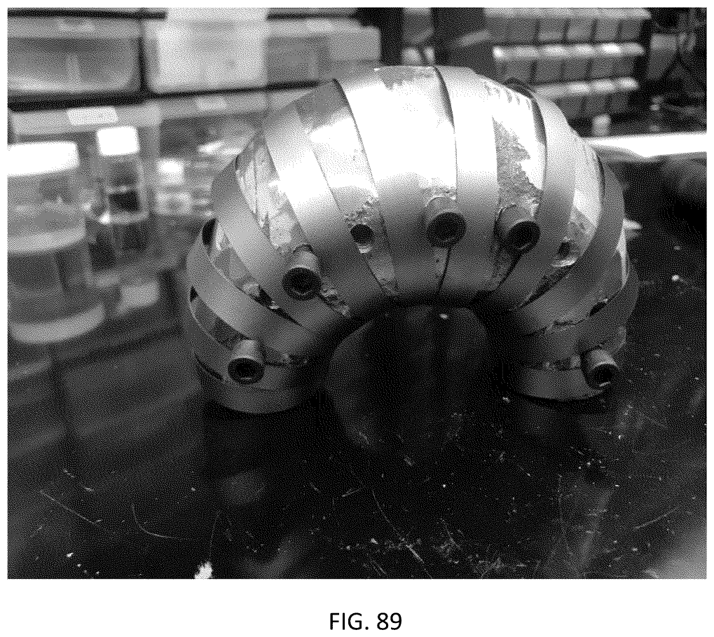

FIG. 90 is an image of a pressure measuring device for a radial stiffness measuring setup according to the disclosure.

FIG. 91 is an image of the radial stiffness measuring setup of FIG. 90.

FIG. 92 is a plot showing an applied pressure curve of a pressure applied a catheter according to the disclosure, using the radial stiffness measuring setup of FIG. 90.

FIG. 93 is a plot showing pressure response curves for three of the pressure sensors of the catheter of FIG. 92.

FIG. 94 is a plot showing calibration data of the all ninety pressure sensors of the catheter of FIG. 92, including the gains and offsets for the conductance-pressure correlation.

FIG. 95 is a plot showing the spectral power of signals in the esophagus where a dilation catheter is used to stimulate a pressure mapping catheter according to the disclosure.

FIG. 96 is a plot showing the spectral power of signals in the esophagus where a bolus balloon is used to stimulate a pressure mapping catheter according to the disclosure.

FIG. 97 is a plot showing a calibration curve of the force sensor for the palpation on a swine abdomen, according to the disclosure.

FIG. 98 is a plot showing an average pressure reading from a catheter according to the disclosure, which is place in a stomach of a swine model during an infusion of Azithromycin.

FIG. 99 is a plot showing pressure measurements taken with the catheter of FIG. 98 during a twenty-five minute infusion of Azithromycin.

FIG. 100 is a plot showing of the pressure measurements of FIG. 99 with a moving average filter applied.

FIG. 101 is another plot showing the pressure measurements of FIG. 99.

FIG. 102 is another plot showing the pressure measurements of FIG. 100.

FIG. 103 is a plot showing a motility map of an esophagus of a swine model that was obtained with a catheter according to the disclosure, where the esophagus was stimulated using a bolus balloon.

FIG. 104 is a plot showing a motility map of an esophagus of a swine model that was obtained with a conventional HRM device, where the esophagus was stimulated using a bolus balloon.

FIG. 105 is a plot showing a motility map of an esophagus of a swine model that was obtained with a catheter according to the disclosure, where the esophagus was stimulated using a dilation catheter.

FIG. 106 is a plot showing a motility map of an esophagus of a swine model that was obtained with a conventional HRM device, where the esophagus was stimulated using a dilation catheter.

FIG. 107 is a plot showing a reflex map of a rectum of a swine model that was obtained with a catheter according to the disclosure, where the rectum was stimulated using a bolus balloon.

FIG. 108 is a plot showing a reflex map of a rectum of a swine model that was obtained with a conventional HRM device, where the rectum was stimulated using a bolus balloon.

FIG. 109 is a plot showing a motility map of a stomach of a swing model that was obtained with a catheter according to the disclosure, where the stomach was stimulated using an infusion of Azithromycin.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The term “about,” as used herein, refers to variations in the numerical quantity that may occur, for example, through typical measuring and manufacturing procedures. Throughout the disclosure, the terms “about” and “approximately” refer to a range of values±5% of the numeric value that the term precedes.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As mentioned above, pressure sensors and transducers can be incorporated into a pressure sensing catheter, (i.e., a GI manometer) that can be used to obtain a pressure profile of a GI tract, or a portion thereof. For example, a manometer can be inserted into endoluminally into an esophageal tract of a patient to measure contractions at various positions along the esophageal tract to produce a pressure profile. The pressure profile can be used by a physician to observe and diagnose GI motility disorders.

Conventional high-resolution manometers generally use anywhere between 21 and 36 solid state pressure transducers that can be inserted into a GI tract of a patient to measure a pressure profile of the GI tract. Each of these pressure transducers typically measure pressure using a Wheatstone bridge, which requires a multiplicity of electrical connections to each individual transducer. Because of the large number of transducers and associated electrical connections, conventional high-resolution manometers are complex and sensitive diagnostic tools that are expensive, difficult to manufacture, and difficult to disinfect. Additionally, because of the large number of electrical connections, conventional high-resolution manometers are generally limited to lengths ranging between 15 centimeters and 80 centimeters, as greater lengths become too bulky to be used in many patients. Accordingly, while conventional systems can provide high resolution and high accuracy pressure profiles, they can only obtain a pressure profile for a limited portion of a gastrointestinal tract at any one time. And further, measurements are typically limited to two-dimensional mapping, limiting use current high-resolution manometry systems to generally tubular portions of a GI tract, such as the esophagus and rectum.

Aspects of the present disclosure can provide for improvements over conventional high resolution manometer systems and other GI mapping systems by providing for a cost effective catheter system for obtaining a pressure profile (e.g., a pressure map) of a gastrointestinal (GI) tract in three dimensions. Additionally, aspects of the disclosure provide for a highly customizable catheter system with performance characteristics that are similar to conventional high-resolution manometry system, while also allowing for increased lengths that permit pressure profiles to be obtained for comparatively large portions of a GI tract, and in some cases an entirety (e.g., an entire length) of a GI tract. That is, catheter systems according to the disclosure can provide a universal motility mapping platform for the GI tract, which can map a three-dimensional pressure distribution inside any of the esophagus, stomach, and rectum.

More specifically, aspects of the invention utilize a catheter (e.g., a flexible piezoelectric device) configured to be inserted endoluminally into a GI tract in order to map a three-dimensional pressure profile of the GI tract, or portions thereof. The catheter can include a sensing unit having a plurality of pressure sensors, which can be supported on a support structure made of a shape memory material (e.g., nitinol or another shape memory alloy, polymer, or composite). The catheter, via the support structure, can have a first configuration at a first stimulus condition (e.g., a first temperature, electrical current, etc.). The first configuration can be selected to ease insertion of the catheter into a GI tract, and may for example be a generally linear configuration for insertion into an esophagus or rectum.

Upon insertion into the GI tract, the catheter, via a shape transition of the support structure, can assume a second configuration at a second stimulus condition (e.g., a second temperature, electrical current, etc.) to conform the sensing unit to the GI tract, thereby positioning the sensing unit and the associated pressure sensors for obtaining a three-dimensional pressure mapping of the GI tract (e.g., for universal mapping of the entire GI tract, or one or more portions thereof). In particular, in some cases, a change in temperature due to body heat from the GI tract can cause the catheter to automatically assume the second configuration. Correspondingly, in the second configuration, the catheter can define one or more regions that are shaped to conform with a specific portion of the GI tract. For example, in a second configuration, the catheter can include a first region that is shaped to conform to a first portion of the GI tract (e.g., an esophagus) and a second region that is shaped to conform to a second portion of the GI tract (e.g., a stomach). The second portion can be distinctly shaped from the first portion of the GI tract. For example, the first region can be linear region or a coiled region with coils of a first diameter and the second region can be a coiled region with coils of a second, different diameter or of a variable diameter. The specific shape(s) attained in the second configuration can be based on the dimensions of the corresponding organs of the GI tract being investigated.

In general, the sensing unit and the support structure can be coupled with one another within an outer sheath (e.g., a flexible overtube made from silicone, silicone-based rubber, latex, polyvinyl chloride, polyurethane, fluoropolymers, thermoplastic elastomers, other types of elastomeric materials, and other materials, including biocompatible and non-biocompatible materials). The outer sheath can define a lumen into which the sensing unit and the support structure can be positioned to maintain a desired spatial relationship therebetween, as well as protecting the sensitive electrical components of the sensing unit. In some cases, the outer sheath can preferably be an electrically-insulative and liquid-tight elastomeric tube, which is biocompatible and has low mechanical hysteresis and a low elastic modulus.

In some cases, the outer sheath can first be inserted into the GI tract, after which the sensing unit and support structure can be inserted into the outer sheath, which can aid in allowing the sensing unit and support structure to be positioned in the GI tract more quickly (e.g., prior to the support structure changing shape at a second temperature condition). Within the outer sheath, the sensing unit can be disposed within a first inner sheath (e.g., a flexible tube) and the support structure can be disposed within a second inner sheath. The inner sheaths can help to ease the passing of the sensing unit and support structure into the outer sheath, as well as protecting the sensing unit and support structure during the insertion process.

A sensing unit of a catheter can include a plurality of pressure sensors that are spaced apart along a flexible printed circuit board (e.g., to be arranged along a length of the catheter). The spacing between the individual sensors can vary depending on the specific application, but can generally have a spacing ranging from about 0.5 cm to about 5 cm or more, and more specifically, about 2 cm. Each pressure sensor is generally formed by a u-shaped portion of the flexible printed circuit board having a first arm portion and a second arm portion, with a compression member disposed between the first and second arm portions. Where multiple pressure sensors are provided, the first arm portions can be connected with one another to form a base portion of the flexible printed circuit board, which can be positioned along a support structure.

The compression member can be made of a resilient material that can be resiliently compressed between the first and second arm portions, which can resiliently move together in response to a force caused by a contraction of a GI tract (e.g., a peristaltic contraction). The compression member can be made of an electrically conductive material, in particular, a porous, anti-static foam material. Consequently, compression of the compression member between the arm portions can induce a change in an electrical property of the compression member (e.g., an increase or decrease). The electrical property may be resistance (or conductance), however, other electrical properties, or inductance or capacitance may be used. In one non-limiting example, a sensor, such as a multimeter or multi-channel measurement device, can be coupled (e.g., electrically coupled) to the tube to measure an electrical property of the compression member (e.g., an electrical resistance of the compression member). The electrical property can be interpreted and correlated with a pressure exerted by the GI tract by the sensor or another a processor, which can then be used to determine a pressure profile of the GI tract.

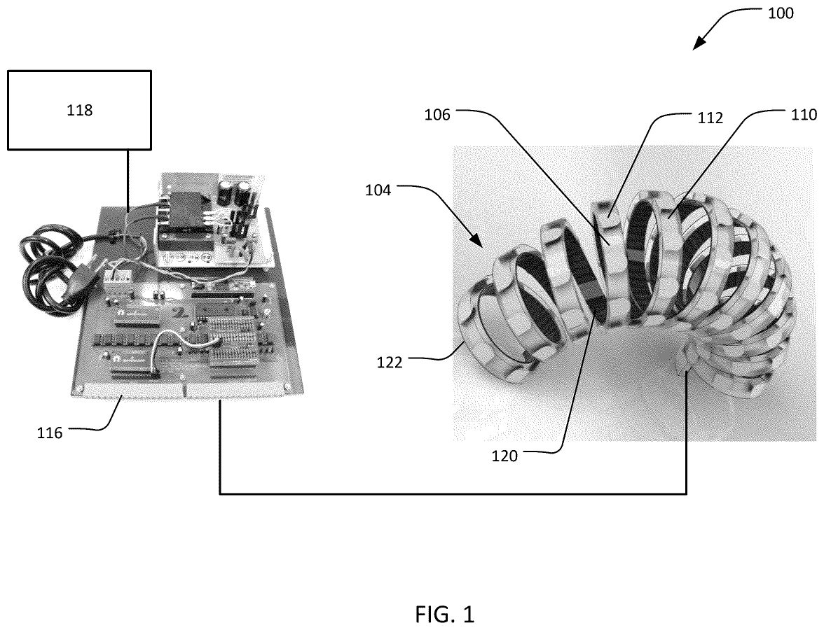

FIG. 1 depicts a non-limiting example of a system 100 (e.g., a catheter system) for obtaining a three-dimensional pressure map of a GI tract, according to aspects of the disclosure. The system 100 is configured as a pressure sensing catheter system having a pressure sensing catheter that can be inserted (e.g., placed endoluminally) into a GI tract to measure one or more pressures within (e.g., produced by) the GI tract, which correspond with a pressure profile of the GI tract. In that regard, the system 100 can be used by a physician to obtain a pressure profile along any portion of a GI tract in order to observe and diagnose GI motility disorders. Thus, the system 100 may have a length that corresponds with the portion of the GI tract to be observed and measured. For example, a length of the system 100 can be configured to correspond with a length of an esophageal tract, colon, or other portion of a GI tract and any combinations thereof, including the GI tract in its entirety. In some cases, as described in greater detail below, the length of the system 100 can be selected to allow for a desired folding or bending of the system 100 within the GI tract to allow the system 100 to be conformal with specific portions of the GI tract.

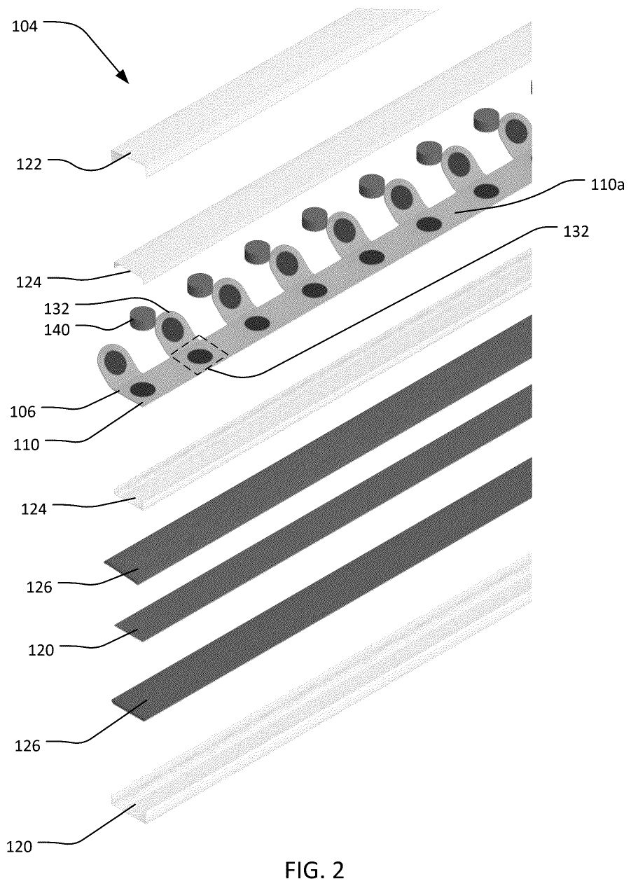

A catheter system can include a pressure sensing catheter that can be configured to conform to a GI tract to measure a pressure or pressure profile. As illustrated in FIGS. 1 and 2, the system 100 includes a catheter 104 having a sensing unit 106 configured as a flexible printed circuit board 110 with a plurality of pressure sensors 112 spaced along a length of the flexible printed circuit board 110. The sensing unit 106 can be in communication with a measurement device 116 that, as will be described in greater detail below, can be configured to can calculate a pressure being applied to the sensing unit 106. In some cases, the system 100 can further include an interface 118 coupled to the measurement device 116, which can display the pressure data or allow a user to interact with the system 100. The sensing unit 106 is supported on a support structure 120 made of a shape memory material, for example an alloy (e.g., nitinol), polymer, or composite shape memory material. As will be described in greater detail below, the support structure 120 can vary in shape to conform to a shape of a GI tract based on stimulus condition.

Referring to FIG. 2, the sensing unit 106 and the support structure 120 can be disposed within a lumen of an outer sheath 122 (e.g., a unitary or two piece outer sheath) that can help to secure the sensing unit 106 and the support structure 120 in a desired spatial relationship with one another. Correspondingly, in some cases, the sensing unit 106 and the support structure 120 can be disposed within the outer sheath 122 to be inserted into a GI tract as a single unit, or the outer sheath 122 can first be inserted into the GI tract and then the sensing unit 106 and the support structure 120 can be positioned in the outer sheath 122, as may help to reduce the amount of time required to place the sensing unit in the GI tract. In some cases, the sensing unit 106 can be positioned within a first inner sheath 124 (e.g., a unitary or two piece sheath) and the support structure 120 can be positioned within a second inner sheath 126 (e.g., a unitary or two piece sheath).

A shape memory support structure can be configured to be in a first configuration at a first stimulus condition to aid in insertion into a GI tract (e.g., a substantially linear configuration) and to transition to a second configuration at a second stimulus condition to be conformal to the GI tract (e.g., to position pressure sensors along the GI tract for pressure mapping). For example, a nitinol support structure can be configured to be in a first configuration for insertion into a GI tract at a first temperature condition (e.g., between approximately 0 degrees Celsius and approximately 30 degrees Celsius) and to transition to a second configuration that is conformal with the GI tract at a second temperature condition (e.g., approximately 35 degrees Celsius). The transition between the first and second configuration can occur automatically due to heat transfer from the GI tract to the support structure. Correspondingly, to reduce the rate of heat transfer and increase the amount of time that a physician has to place the catheter in the GI tract, multiple sheaths can be used (e.g., the outer sheath 122, and first and second inner sheaths 124, 126).

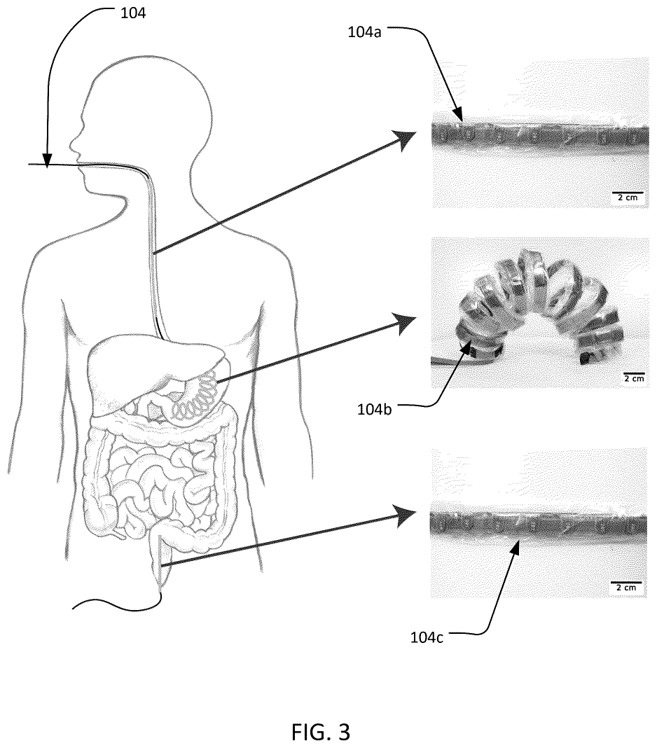

In some cases, a catheter can be configured with one or more regions that are selectively shaped to conform with a specific portion of a GI tract upon transitioning from a first configuration to a second configuration. For example, as illustrated in FIG. 3, in a second configuration that is attained following insertion into the GI tract, the catheter 104 can define differently shaped regions that are each configured to conform with a specific portion of the GI tract, thereby placing the pressure sensors 112 to acquire pressure readings in three dimensions. In particular, the catheter 104 defines a first region 104a corresponding with the esophagus, a second region 104b corresponding with the stomach, and a third region 104c corresponding with the rectum. The first region 104a and the third region 104c are configured as linear regions in accordance with the tubular nature of the esophagus and rectum. However, the second region 104b is configured having a coiled (e.g., spiral shape) to form a conformal bolus that places the pressure sensors 112 along the stomach walls. That is, the coil shape can be configured so that the pressure sensors 112 are positioned along an exterior periphery of the coil to engage with the walls of the GI tract, while the support structure 120 is positioned along an interior periphery of the coil. To account for variability in the shape of the GI tract and allow for peristaltic contractions, the flexible nature of the support structure 120 can allow for stretching, bending, and compression of the regions of the catheter 104 to conform with the GI tract. Further, regions with other types of shapes can also be used.

As mentioned above, a catheter can be a piezo-resistive catheter that is configured to be compressed by the GI tract to produce a change in one or more electrical properties of the catheter 104. This change in electrical property can be used to determine the pressure acting on the catheter 104. For example, when the catheter 104 is inserted into a GI tract, the peristaltic contractions of the GI tract compress the catheter 104, creating a change in electrical properties, such as resistance (e.g., via a piezo-resistive effect), which can be correlated with a pressure produced by the GI tract. More specifically, as illustrated in FIG. 1, the pressure sensors 112 are provided along the flexible printed circuit board 110 and the change in electrical resistance can be correlated with the pressure acting on the one or more pressure sensors 112.

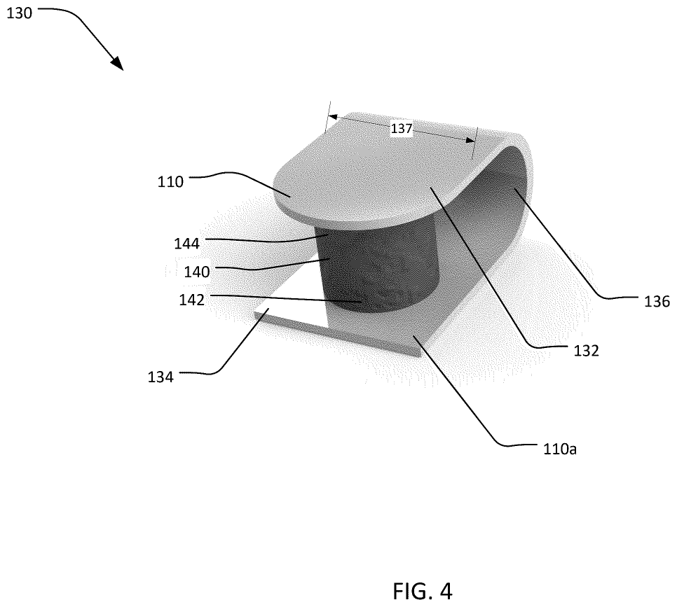

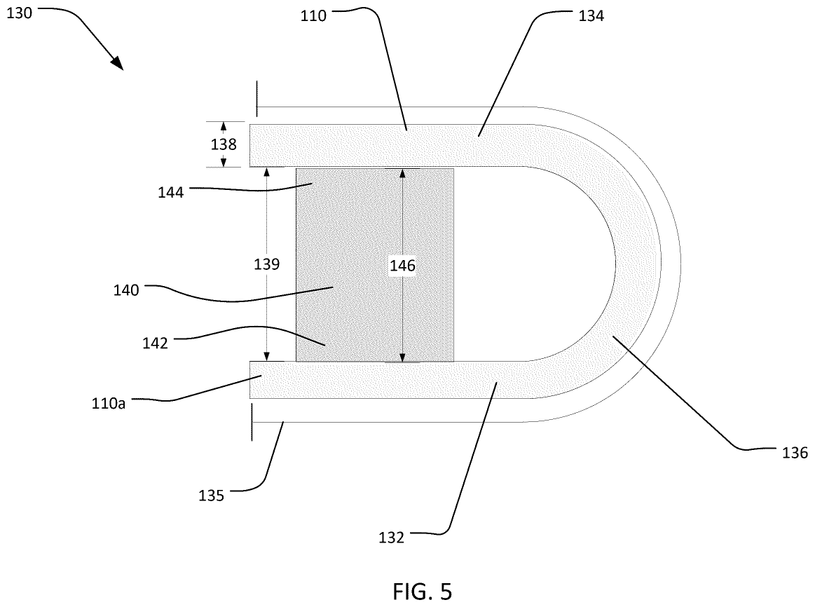

Referring now to FIGS. 4 and 5, a pressure sensor 130 of the plurality of the pressure sensors 112 is shown. Accordingly, discussion of the pressure sensor 130 applies equally to the others of the plurality of pressure sensors 112. In general, the pressure sensor 130 includes a u-shaped portion of the flexible printed circuit board 110. As used herein, unless otherwise specified or limited, the term “u-shaped” specifies a shape that includes a base portion, with at least two leg portions extending in similar directions from the base portion. In some embodiments, a “u-shaped” member can include leg portions extending from opposite ends of the base portion at substantially right angles to the base portion (i.e., deviating from right angles by less than 5 degrees), with or without curved, chamfered, or otherwise non-square connecting regions between the leg portions and the base portion. In some embodiments, a “U-shaped” member can include leg portions that extend as part of a continuous (e.g., non-angled) curve from either end of a straight or curved base portion. Thus, for example, some “u-shaped” members may have half-rectangular, semi-circular, or other similar cross-sectional profiles. Further, some “u-shaped” members may include a first leg that is longer than a second leg. In some cases, a base portion of a u-shape can extend over a circular segment.

Correspondingly, the pressure sensor 130 includes u-shaped portion having a first arm 132 and second arm 134 (e.g., first and second arm portions of the flexible printed circuit board 110) that extend from a base portion 136. Where multiple pressure sensors are provided on a single circuit board, the first arm portions can be portions of a main body of the flexible printed circuit board. For example, as illustrated in FIG. 2 the first arm 132 can be a portion of a main body 110a of the flexible circuit board 110 and the second arm 134 and the base portion 136 can extend from the main body 110a. The main body 110a is configured to be supported on the support structure 120.

Due to the flexible nature of the flexible printed circuit board 110 material, the first and second arms 132, 134 can be resiliently compressed to move close to one another when acted upon by an external force, for example, a contraction of a GI tract. Correspondingly, the u-shaped portion can define a length (l) 135 extending along the arms 132, 134 and base portion 136 between the distal ends of the arms 132, 134, a width (w) 137 taken perpendicular to the length 135, a thickness (t) 138 corresponding to the thickness of the flexible printed circuit board 110 material, and a height (h) 139 corresponding the gap between the distal ends of the arms 132, 134.

Still referring to FIGS. 4 and 5, a compressible member 140 is disposed between the first and second arms 132, 134 and can be resiliently compressed by the arms 132, 134 as they move closed to one another due to an external force. More specifically, a first end 142 of the compressible member 140 can be coupled to the first arm 132 at a first contact area and a second end 144 of the compressible member 140 can be coupled to the second arm 134 at a second contact area. The compressible member 140 can be secured to the arms 132, 134 by an adhesive (e.g., double sided-tape, not shown). As illustrated, the compressible member 140 has a cylindrical shape and defines a height (h_{f}) 146 and a cross-sectional area (A_{f}) taken perpendicular to the height. In other cases, the compressible member can have other shapes.

The compressible member 140 can be formed from an electrically conductive material, in particular and an anti-static foam or other porous, resiliently compressible material. An electrical property (e.g., electrical resistance) of the compressible member 140 can change in response to an external force acting to move the arms 132, 134 together, thereby compressing the compressible member 140. This effectively reduces the height 146 of the compressible member 140, which can cause the electrical property of the compressible member 140 to change (e.g., to increase or decrease). The change in electrical property of the compressible member 140, as measured across the first and second arms 132, 134, can then be correlated with the pressure acting on the pressure sensor 130.

Because the u-shaped portion of the circuit board 110 and compressible member 140 are both resilient members that are compressed together, they act as two springs connected in parallel. As described in greater detail below, the compression characteristics of the pressure sensor 130 can be modeled using a twin spring mathematical model.

### Experimental Results

A universal motility mapping system for the GI tract was developed (e.g., the system 100), which can map 3D pressure distribution inside the GI tract, including the esophagus, stomach, and rectum. The system is configured to be used in pressure mapping of different parts of the GI tract, which can vary significantly in size and shape. That is, the system is able conform to anatomic and dimensional variation across subjects, and demonstrates the ability to evaluate changes in pressure within the stomach, esophagus, and anorectal canal. To achieve measurements of pressure in these distinct areas we employed a shape memory metal (Nitinol)-support coupled to sensing arrays. The motility sensing probes (e.g., catheter 104) uses a Nitinol base structure, a shape memory alloy, for its conformal and adaptive supporting structures, which is designed according to the anatomic shape/dimension of the GI tract. The probes can be stretched into a long (linearized) belt and delivered into the body via the esophagus or rectum. Once the probe is deployed, body heat triggers the phase transition of the Nitinol supporting structure; it then turns into the pre-programmed shape.

The design of the supporting structures is based on the dimensions of the organs from the target cohort of subjects. In the examples below, some dimensions used are based on the size of the stomach of the swine model. More generally, the Nitinol support can be personalized for a particular application depending on body habitus of the subject. An array of pressure sensors (e.g., the pressure sensor 112 of the sensing unit 106) is embedded into the probe, with a spatial resolution of 2 cm. Once the phase transition is finished, the pressure sensors are delivered to the wall of the GI tract. After the testing, the probes can be pulled out from the subject without surgical procedure. During an in vivo test in an adult swine model, the total delivery/retrieval process took around 10 min. The platform has been tested to record motilities/reflexes in the esophagus, rectum, and stomach in adult swine. A clinically used and FDA-approved, high resolution manometry (HRM) system was used to compare the 2D data obtained from the esophagus and rectum. Esophageal peristalsis data from the platform and the HRM exhibited significant similarity. The raw signals recorded from the probes and the HRM have shown a mean correlation coefficient of 0.55 and a p-value of 1E-5. The power spectra of the motility signals have shown a correlation coefficient of 0.62. The platform was able to detect a signal of the rectoanal inhibitory reflex in the rectum. The platform's three-dimensional pressure mapping ability was validated by the close correlations on timing, spatial distribution features between the forces applied on the animal and the data recorded from the probe, in the esophagus, stomach and rectum.

### Universal Motility Mapping System

The system developed in this work had three different probes that are designed to map the mechanical pressure in stomach, esophagus, and rectum, respectfully. It is appreciated that, in other non-limiting examples, the probes can be combined into a single probe with each probe corresponding to a region of the single probe that is adapted to pressure map a particular section of a GI tract (see e.g., FIG. 3). For example, linear sensor belts are used for tubular organs like esophagus and rectum. To map the 3D distribution of the mechanical pressure in the stomach, a conformal bolus supporting structure was envisaged and built (region 104b in FIG. 3). The probes have a balanced stiffness and flexibility which allow them to stand, be stretched, be crushed, or be extended for deployment purposes (see e.g., regions 104a, 104c in FIG. 3). Each of the probes has a linear pressure sensor system composed of a flexible printed circuit (FPC), a nitinol strip as the supporting structure, and two layers of sealing to make the devices liquid tight (FIG. 2). In particular, to deploy the stomach probe, it can be stretched into a linear belt and pushed through the overtube (e.g., outer sheath 122) placed inside the esophagus.

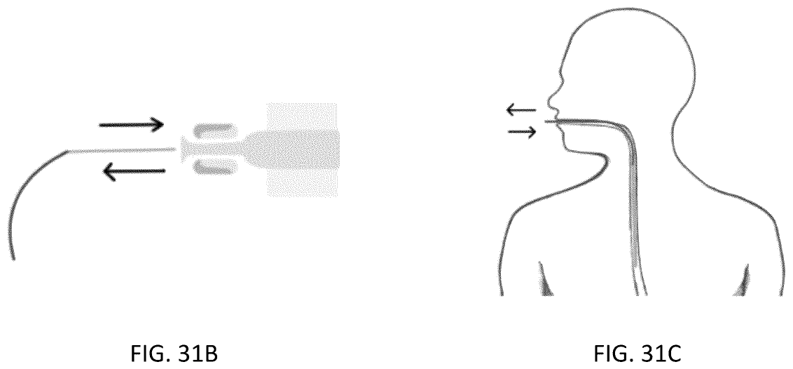

Once the device is inside the body, the body heat from the subject triggers the nitinol phase transition. The probe returns to the programmed shape and becomes conformal to the stomach. The protocol to program the nitinol is discussed, below. In the retrieval process, a cable attached to the probe can be pulled, and the probe will come out via the overtube in the esophagus. These steps are shown in detail in FIGS. 31A-C.

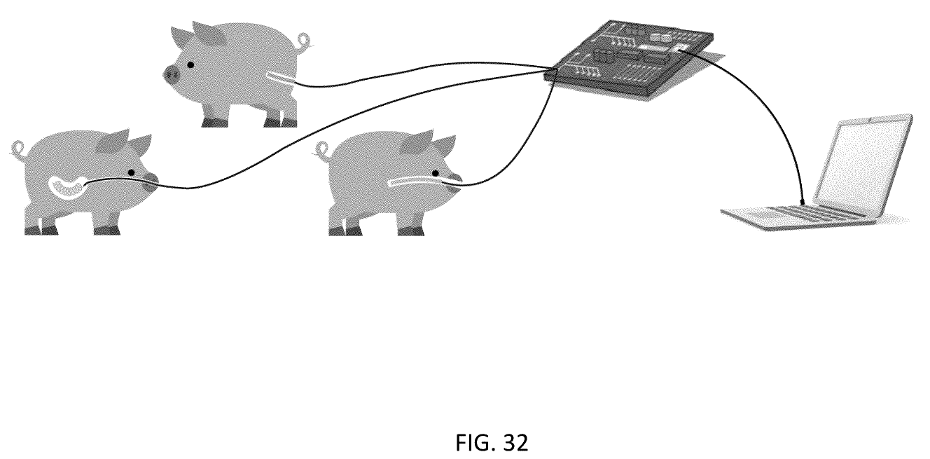

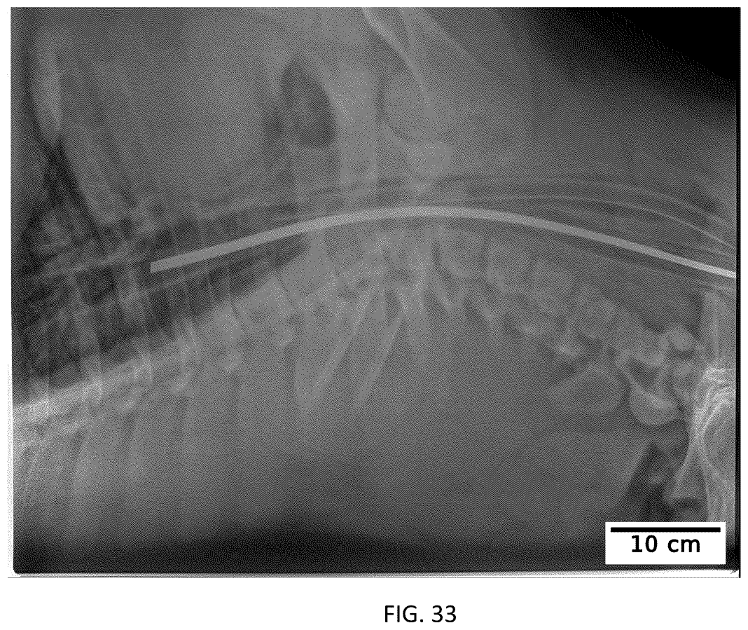

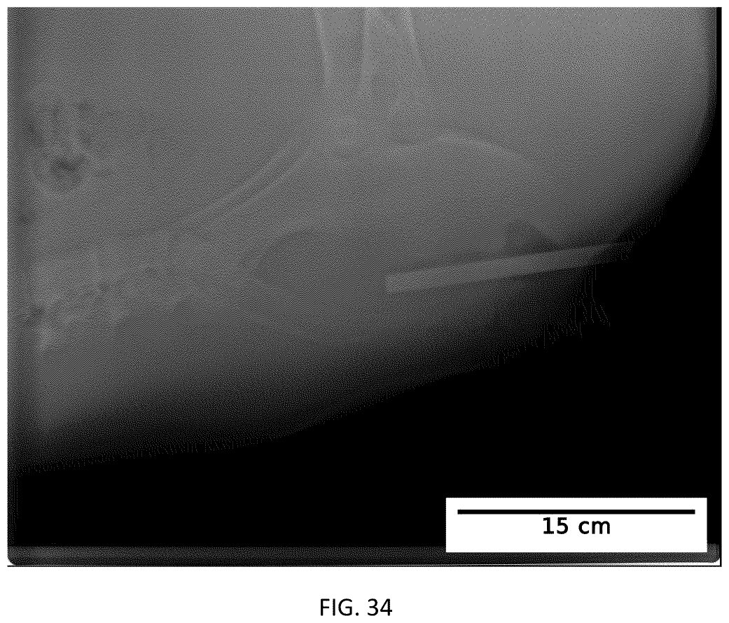

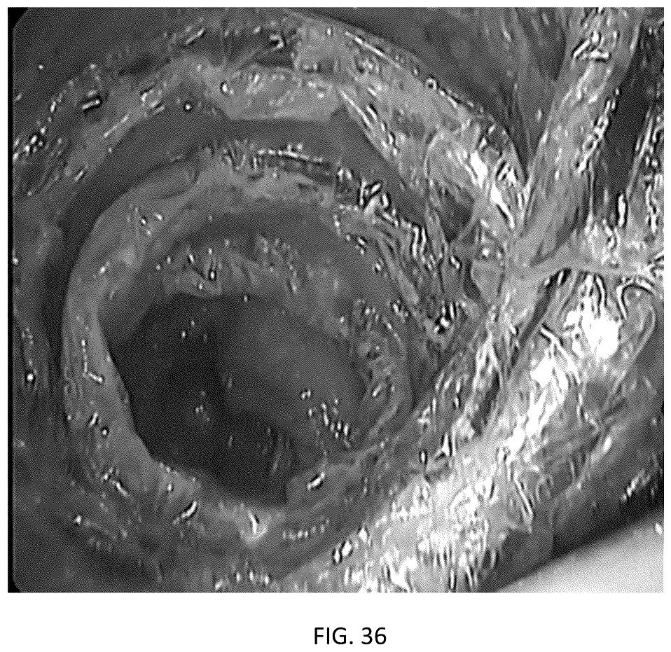

In accordance with the disclosure, rectal and esophageal probes can be directly inserted into the rectum or via an overtube into the esophagus, respectively. Cables attached to either rectal or esophageal probe can be withdrawn; the probes then exit the subject. To validate the proposed concept, Yorkshire swine weighing 75-100 kg was used as the platform. During the experiments, all three probes are deployed into the swine, and an electronic connection cable transmits the resistance change of the individual pressure sensors to the scanning interface. The interface, followed by data streaming into a computer (see FIG. 32). In FIGS. 33-35, it is illustrated with the X-rays of the probes in the esophagus, rectum, and stomach, respectively, and the endoscope image of the stomach device deployed to the swine model is shown in FIG. 36.

### Sensing Material

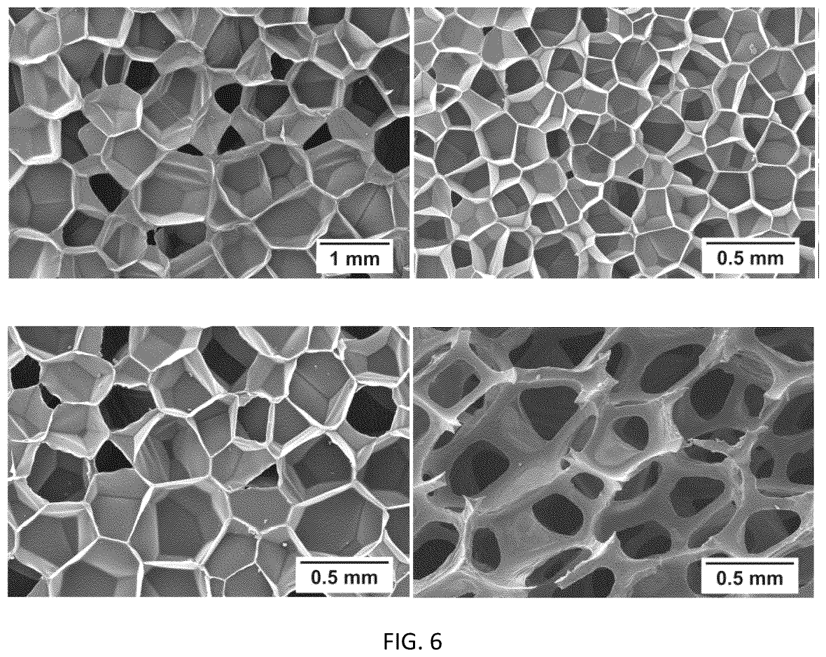

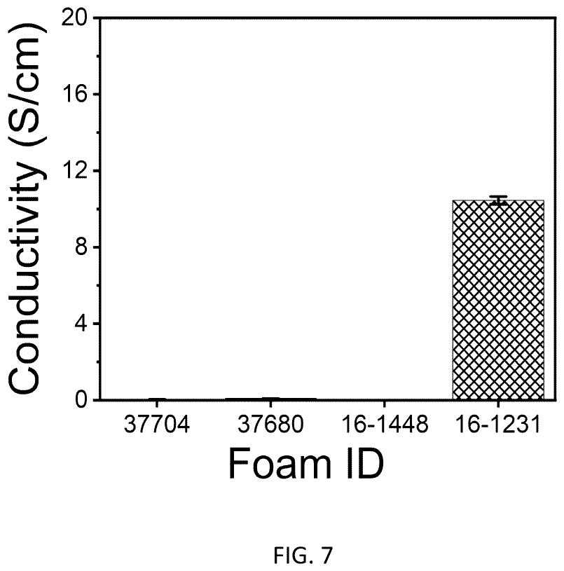

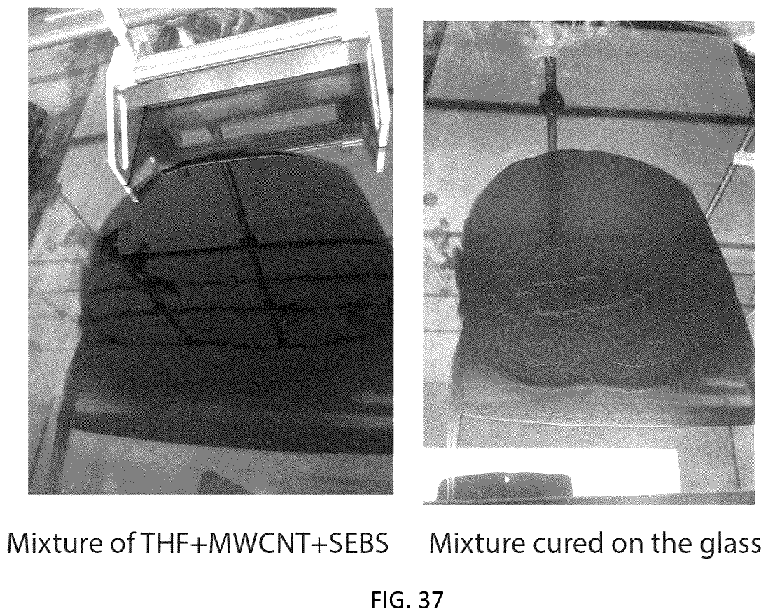

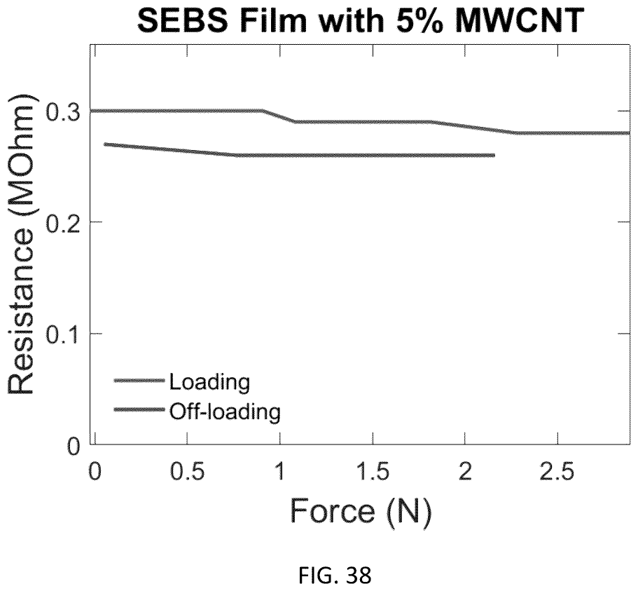

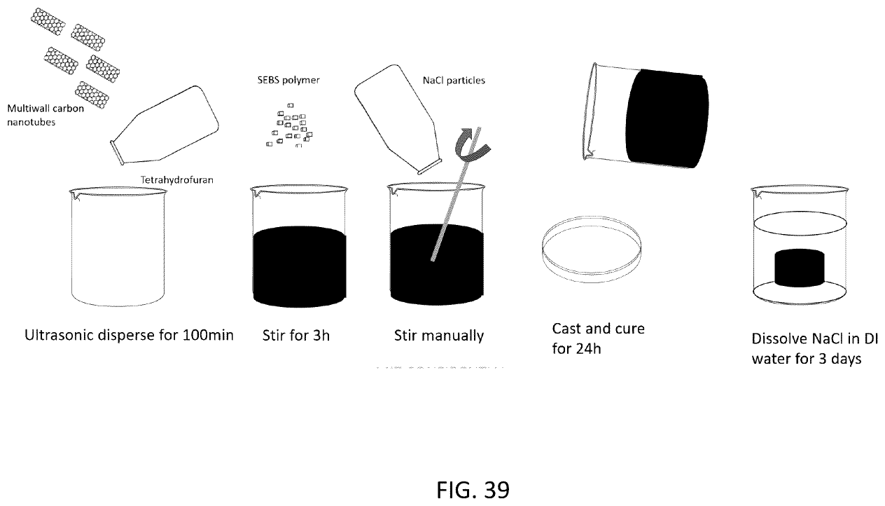

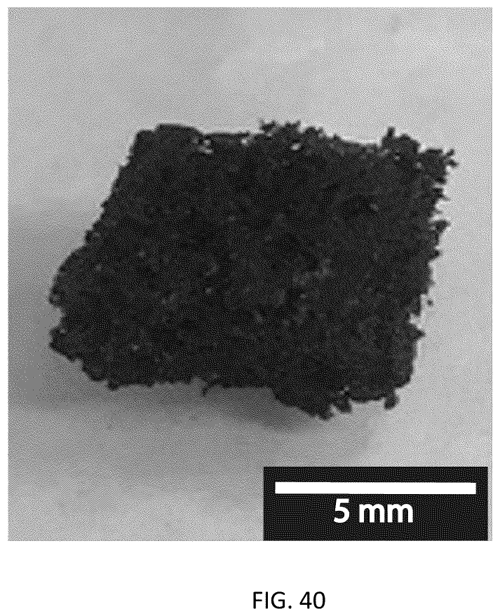

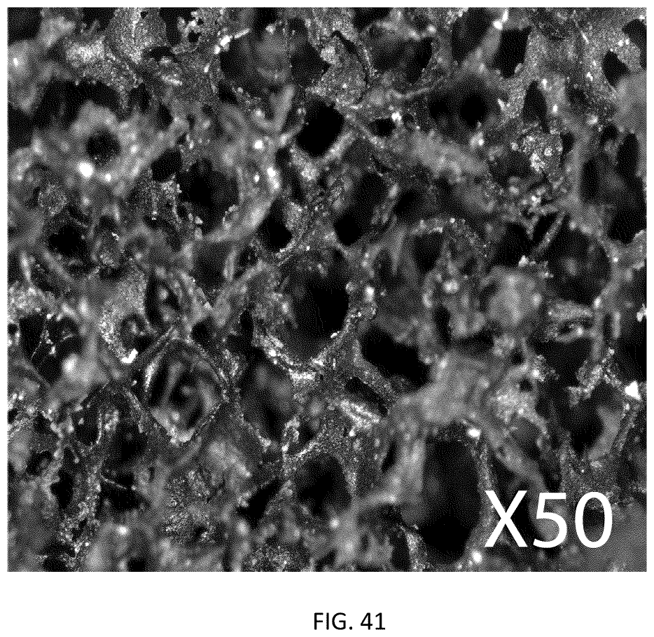

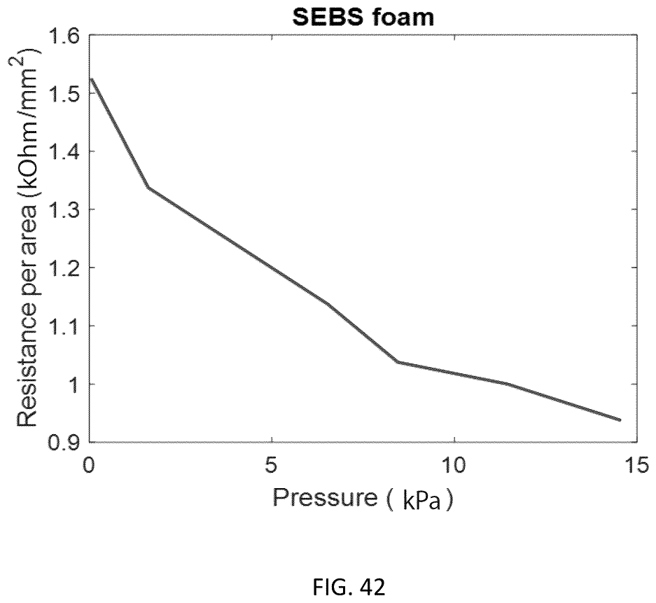

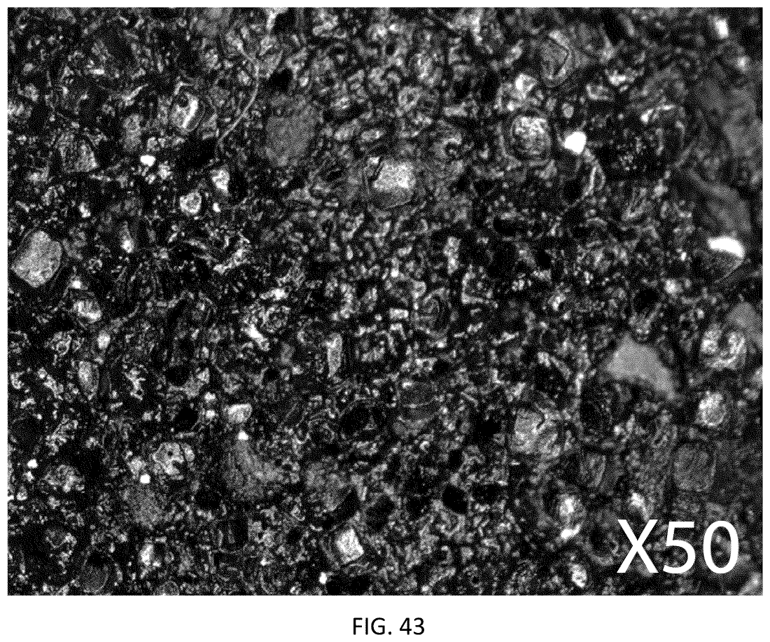

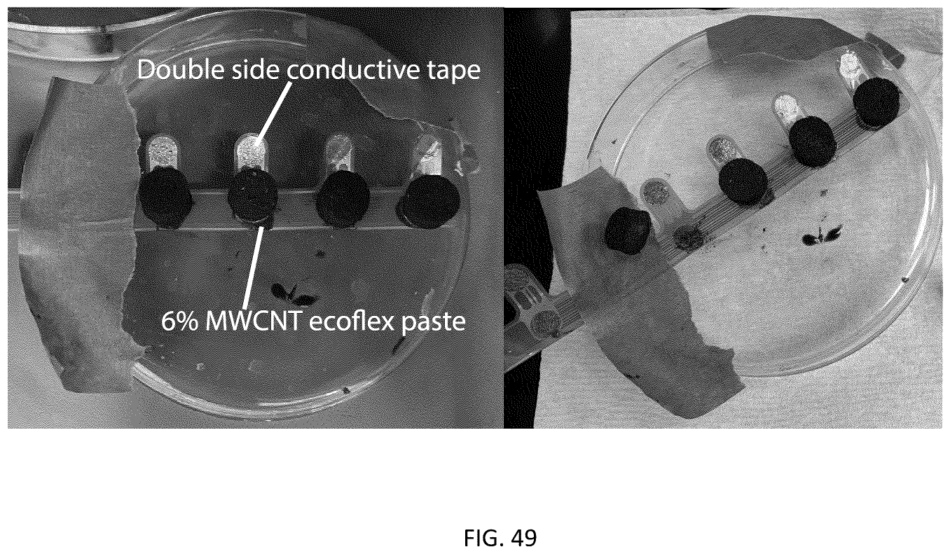

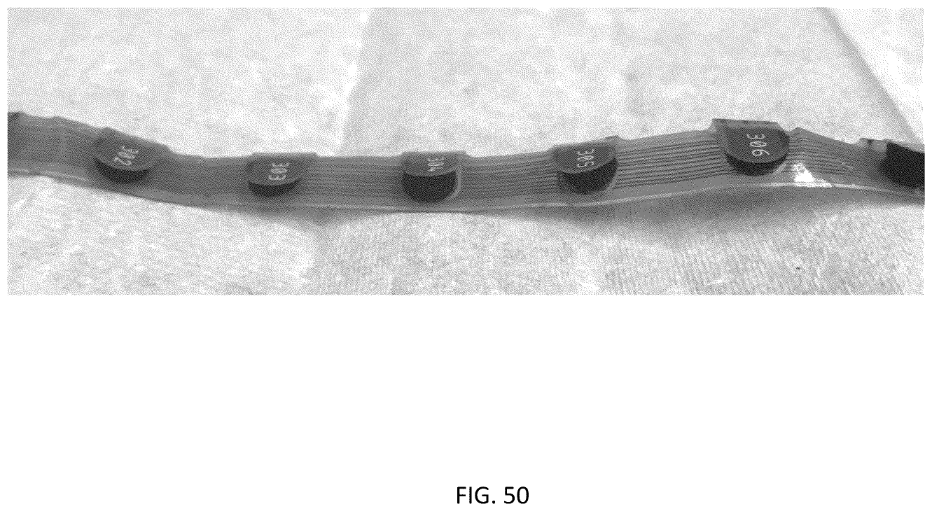

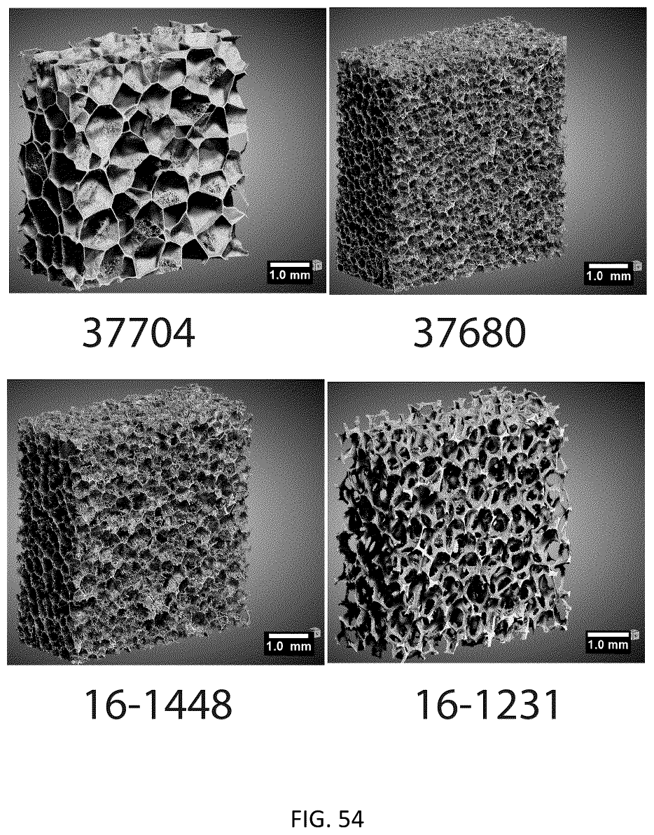

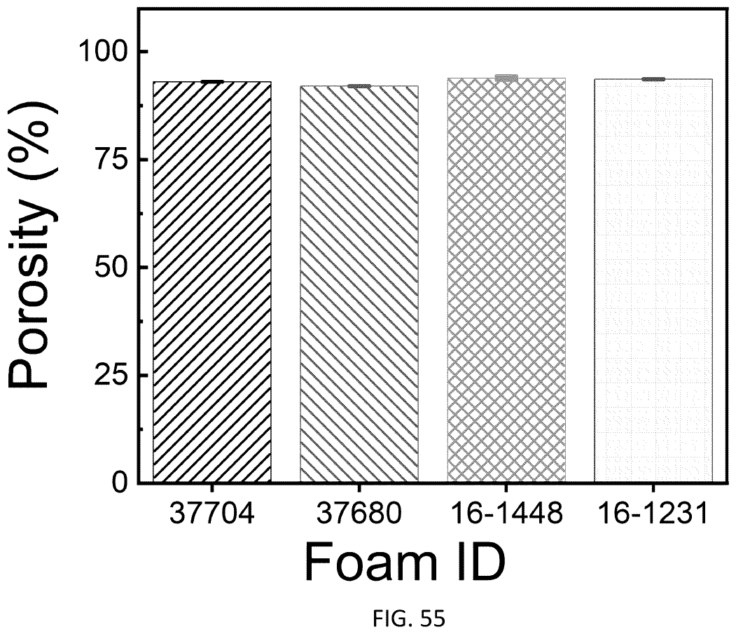

A pressure sensor was built using commercially available anti-static foams and fabricating Multiwall Carbon Nanotubes (MWCNT) doped polymers (see FIGS. 37-50 illustrating a process of fabricating MWCNT doped polymers). An anti-static foam was ultimately used as the sensing material due to its combination of mechanical, electrical, and electromechanical performance, and good adhesion with the FPC circuit board, as well as these foams being readily available and inexpensive to procure. Four kinds of off-shelf foams were used in the screening process (Digikey. Inc., part number: 37704-ND, 37680-ND, 16-1448-ND, and 16-1231-ND). For each foam sheet, three pieces each with a diameter of ½ inch were punched out. As can be seen from the scanning electron microscopy (SEM) images in FIG. 6, all four foams exhibit porous structure with the pores' diameter being approximately 1 mm to 0.5 mm. The 3D morphology was evaluated by using a 3 μm resolution CT-scan 3D file. (FIG. 54) It revealed for all the tested foams, a 3D structure with over 90% porosity. (FIG. 55) Furthermore, the conductivity of the sample 16-1231 was higher than the other foam samples, presenting a value of 10 S/cm (FIG. 7). As seen in FIG. 7, foam 16-1231 has a conductivity of 10 S/cm, while other foams have significantly smaller conductivity.

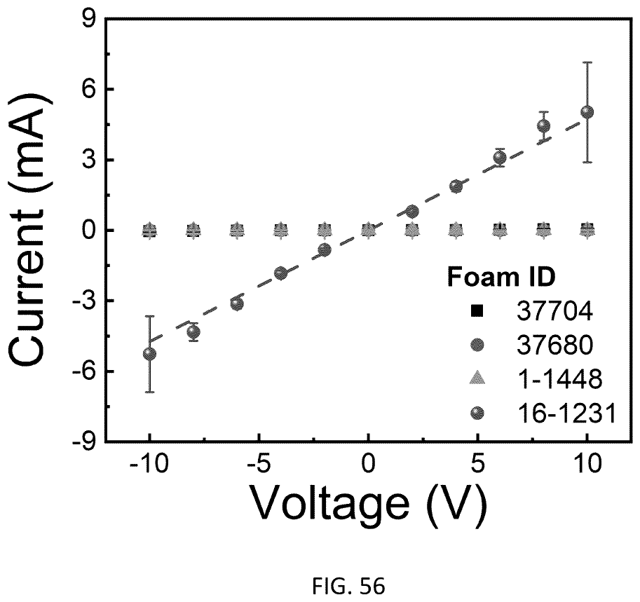

A sourcemeter (Keithley 2450) was used to apply DC voltages across the foams and measure the currents. The I-V curves of four foams are shown in FIG. 56. Foam 16-1231 shows a linear I-V relationship. Its current changes from −5 to +5 mA when the voltage changes from −10 to +10 V. The other three foams show comparatively little variation of currents with −10 to +10 V applied on. By utilizing the data from I-V curves, the conductivity of the foams was obtained.

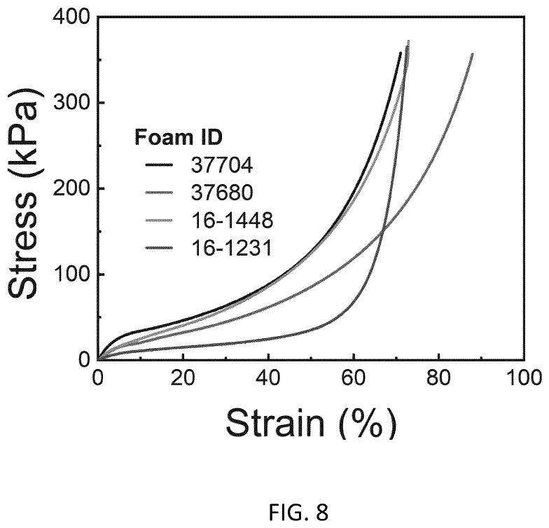

The foams were loaded on an Instron (Universal Testing Machines) to study their mechanical properties (FIG. 8). A maximum pressure of 350 kPa was applied to the foams. All foams exhibited three phases of deformation (e.g., a stress-strain relationship). In the first phase, we observed a linear strain change with stress increase. In the second phase, a plateau was observed with a large strain change along with relatively small stress increase. While in the third phase, the stress increases exponentially with very little strain change. This is due to the pore collapse and foam densification that occurs at higher strains.

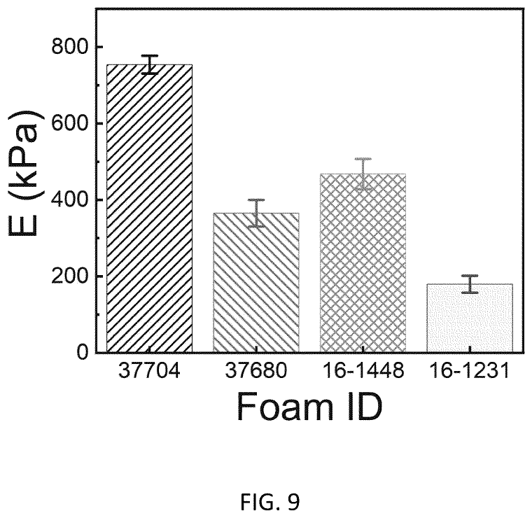

The foam 37704-ND presents the highest value for the Young's modulus (E=754±24 MPa), while the softer foam (16-1231-ND) shows a E=180±22 MPa (FIG. 9). To design the universal GI tract motility mapping system device based on the piezoresistive mechanism, samples with low elastic modulus and higher electrical conductivity were used. The results gathered for the different foams, showed that foams with lower E (FIG. 9) and higher electrical conductivity (FIG. 7) achieved higher performance.

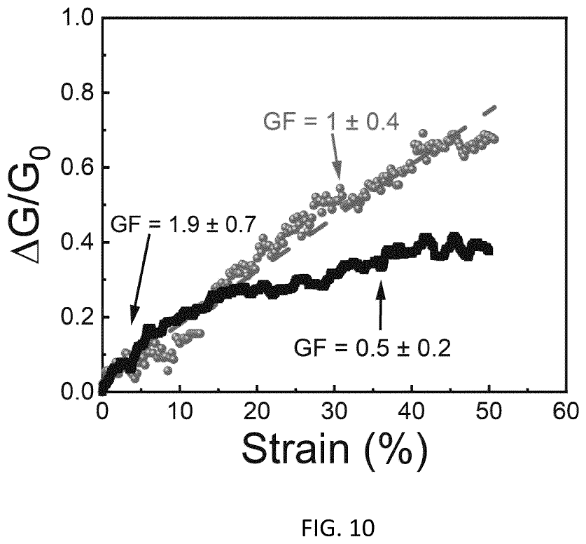

To evaluate the conductive foam electromechanical properties, two electrodes were connected to the top and bottom part of the sample and the change in resistance was recorded when a mechanical compressive stress was applied to the porous material (FIG. 10). During the anti-static foam compression, the walls of the pores and possibly the distance between electrically conductive fillers decrease, creating more pathways for electrons to migrate, leading to an overall increase in sample conductance with applied mechanical strain. The Gauge Factor

$$( GF = \frac{\frac{Δ G}{{G}_{0}}}{ε} ,$$

where

$$\frac{Δ G}{{G}_{0}}$$

is the normalized change in electrical conductance, and ε is the applied mechanical strain). The electromechanical behavior of the foam 16-1231-ND was assessed by applying a mechanical input at different stroke speeds, followed by monitoring the change in electrical conductance. It was observed that the samples presented a two linear regions for the experiment performed at a stroke speed below 20 mm/min, with a GF value of 1.9±0.7 for the first linear region that goes up to 10% strain, followed by a drop down to 0.9±0.2 up to 50% deformation. On the other hand, when the stroke speed is 50 mm/min, a linear relationship between the foam deformation and the

$$\frac{Δ G}{{G}_{0}}$$

was observed, and a GF value or 1.0±0.4 was calculated (FIG. 10).

### Single Sensor Cell Design

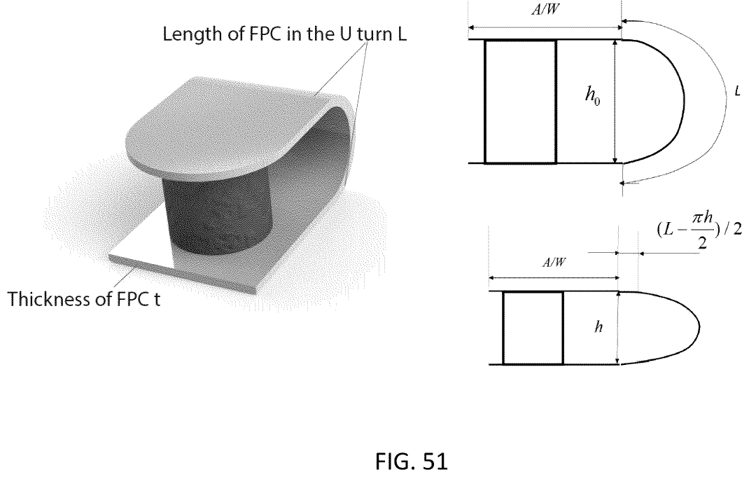

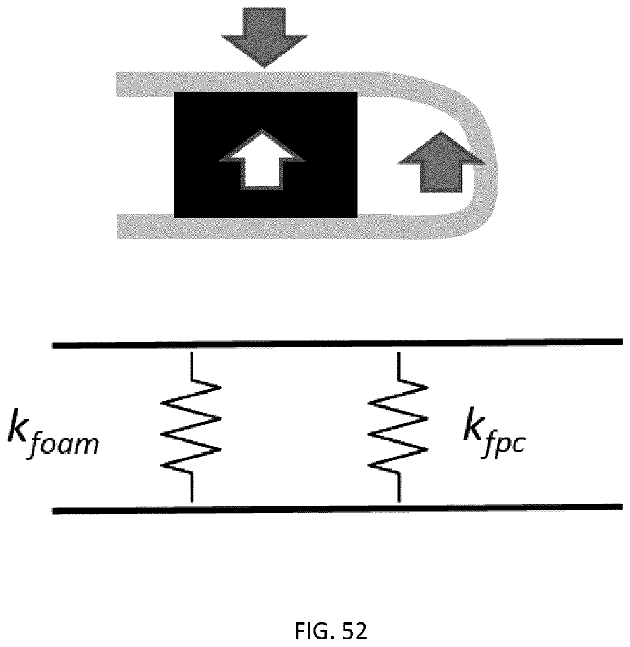

To design the pressure sensor, two variable parameters were investigated according to manufacturer's capacity, thickness of FPC, t, and length of the U-turn, L. (FIG. 51) A twin spring model was developed to understand the mechanical performance of the sensor (FIG. 52). Each turn in the supporting structure is modeled as a helical spring. Each turn is compressed because of the weight of the sensing probe. Further details of the model are described below.

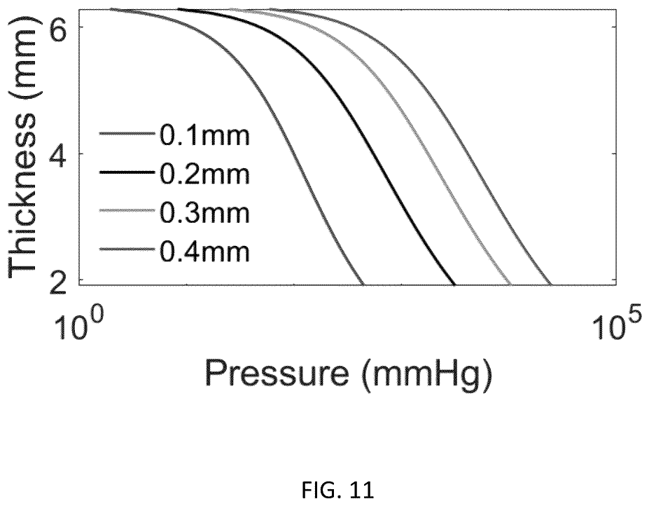

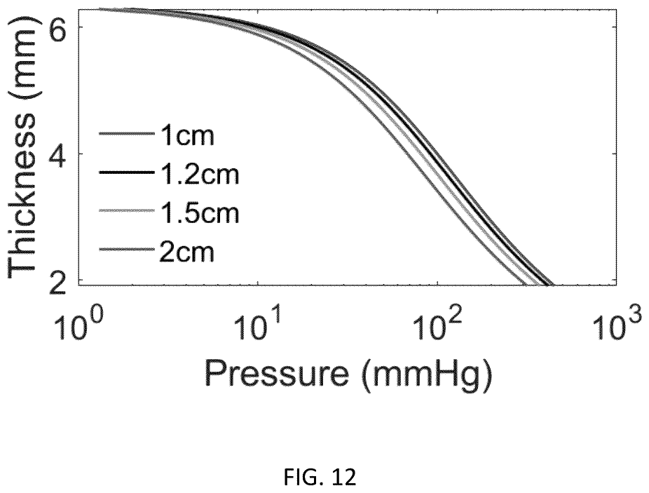

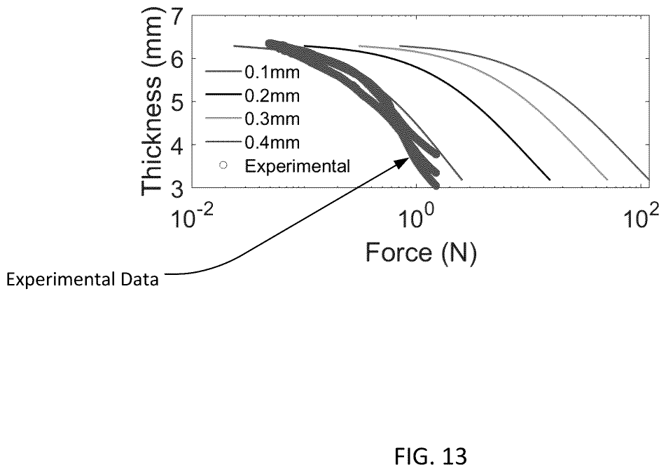

The manometry measurement shows that the motility may result in a pressure change of 250 mmHg, thus a soft material with a large thickness change of the sensor for a pressure difference in the range of 1-250 mmHg was desired. As shown in FIG. 11, within the pressure range of 1-250 mmHg, only the t=0.1 mm case shows a large sensor thickness change as of 2 mm. The other three cases of t=0.2, 0.3, and 0.4 mm have little to no thickness change, suggesting that the sensors are too mechanically stiff to reflect the pressure change. Using t=0.1 mm while changing L, the tested cases had similar pressure-thickness relations, as can be seen in FIG. 12. This indicates the stiffness of the sensor is in-sensitive to U turn length L. To verify this model, the pressure applied on the sensor was converted into force (N) and plotted sensor thickness under different forces in FIG. 13). The thick line is made up from the experimental data where a sensor cell with t=0.1 mm and L=1 cm was compressed with three strokes on a mechanical tester apparatus.

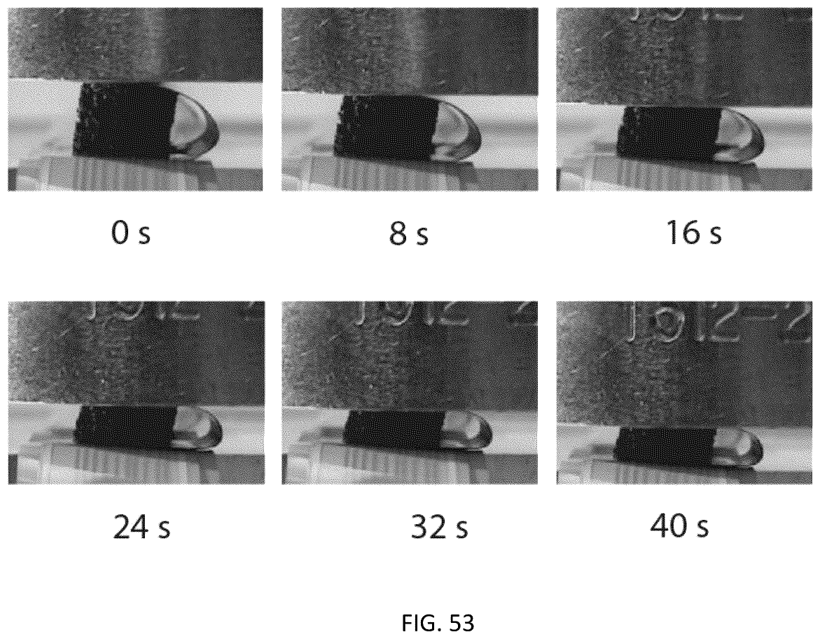

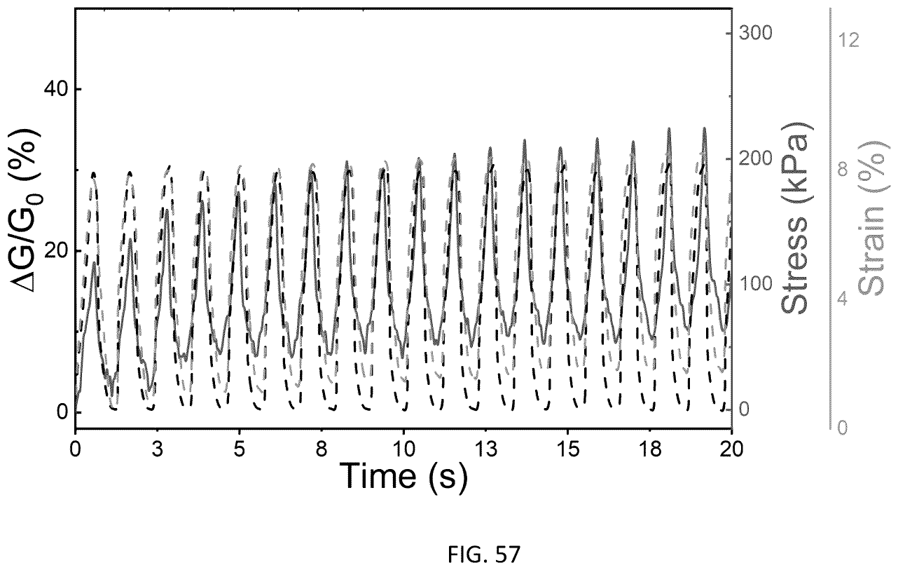

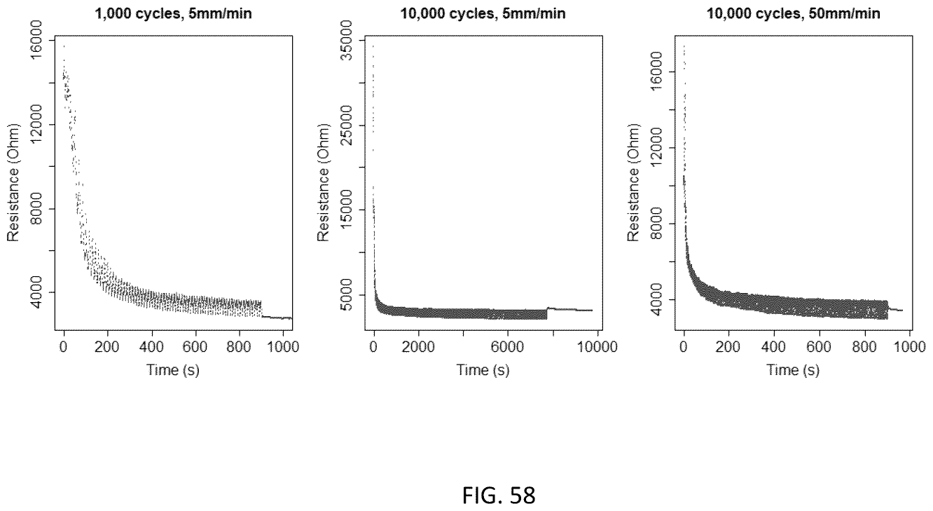

The model and experimental data showed close alignment in the t=0.1 mm case. Based on these tests, optimal parameters were found to be foam 16-1231, t=0.1 mm and L=1 cm. The model was further verified by observing the mechanical behavior of the sensor cell (FIG. 53). When the sample is compressed, the foam cell shrinks in height and behaves like a linear spring, and the diameter of the U turn gets smaller. This behavior is in accordance with the twin model prediction. The dynamic response of the sensor cell of a cyclic compression was studied with a period of 1 s. As can be seen in FIG. 57, conductivity, stress, and strain show synchronized variations. Given the period of peristalsis/reflex in the GI tract is approximately 10 seconds, the sensor showed adequate response speed. A life-time tests of sensor cells was also conducted with a stroke speed of 5 mm/min for 1000 cycles, 5 mm/min for 10,000 cycles, and 50 mm/min for 10,000 cycles (FIG. 58). The life-time test suggest the sensor will be able to operate for at least 24 hours at an in vivo setting, given it survived 10,000 cycles and the period of contract in the GI tract is approximately 10 seconds.

### Electronic Scanning Board

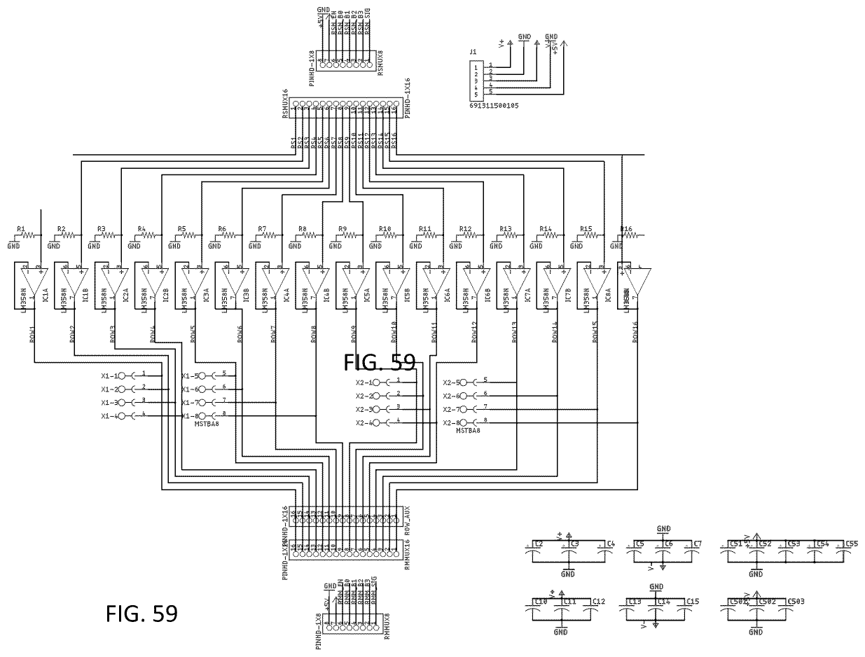

Pressure sensing foam and reference resistors are electrically arranged in an array with row and column connections. A driving voltage is applied to each row sequentially, and the resulting current is converted to a measurable voltage by a bank of transimpedance amplifiers, sampled, and sent to a host system via USB Serial connection. The printed circuit board of the pressure mapping system and its power supply is mounted on an acrylic board (see e.g., measurement device 116 in FIG. 1). The printed circuit board consists of a motherboard, which hosts the microcontroller, amplifiers and multiplexers, and interchangeable daughterboards bearing reference resistors and transimpedance feedback resistors (see FIGS. 59-64).

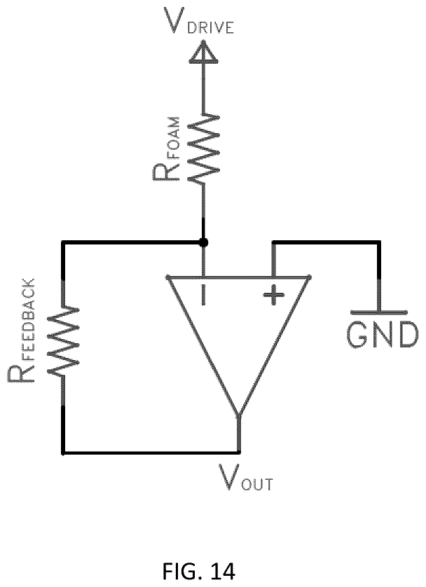

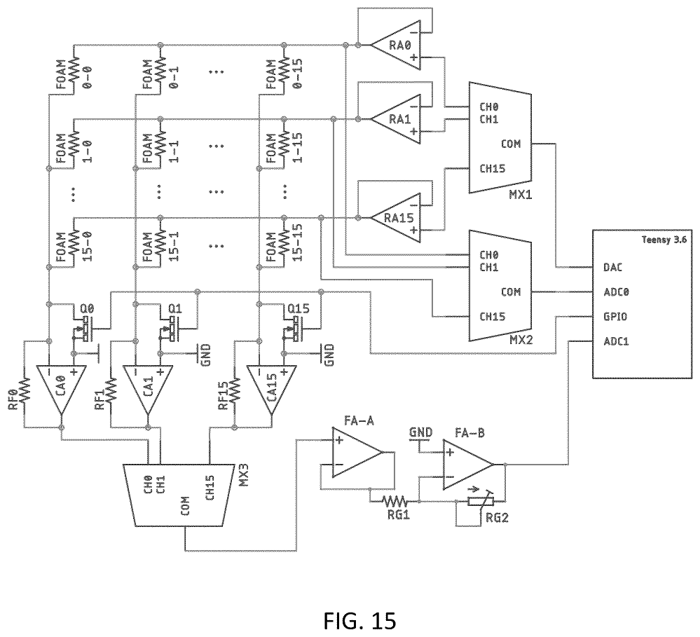

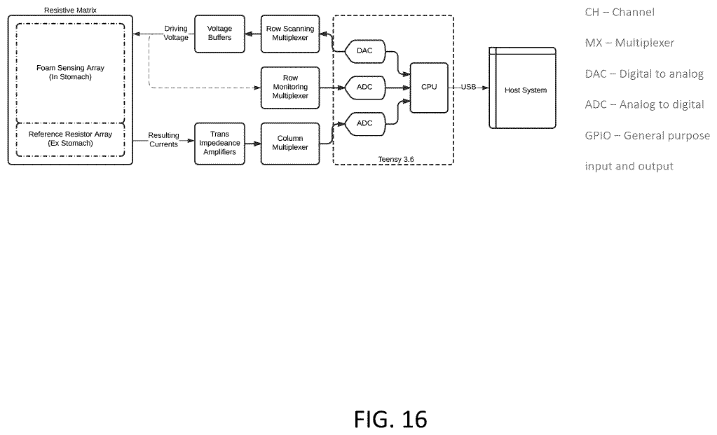

The governing principle of resistance measurement through a transimpedance amplifier is illustrated in FIGS. 14 and 15. The output voltage of the transimpedance amplifier is described by Equation 1, below. To measure the resistance of all sensing elements in the array, “zero potential scanning” was used. As shown in FIG. 16, a voltage buffer is connected to each row, and a transimpedance amplifier is connected to each column. By controlling the multiplexer, a known driving voltage is applied to one row connection while leaving other row connections at the ground potential. The transimpedance amplifiers on each column ensure all columns are also at ground potential. Thus, sensing elements on the row to which driving voltage is applied have current through them, while sensing elements on other rows are at the ground potential. This method ensures that the resistance calculated from driving voltage and resulting current is not affected by bypass current through sensing elements on non-driven rows, known as “cross-talk”.

$${V}_{out} = - \frac{{R}_{feedback}}{{R}_{foam}} {V}_{drive} \qquad Equation 1$$

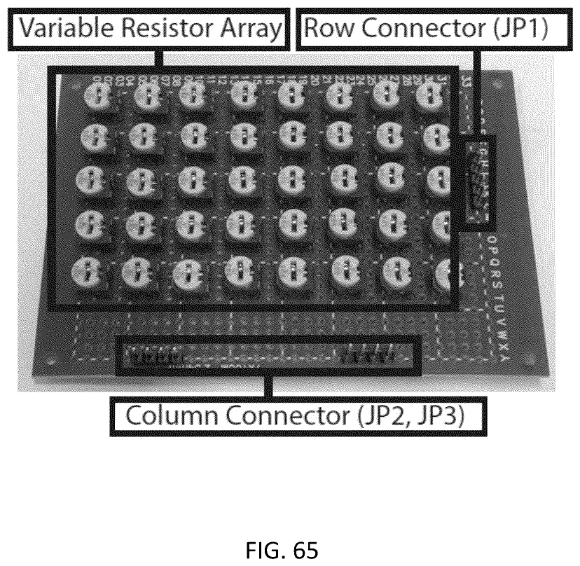

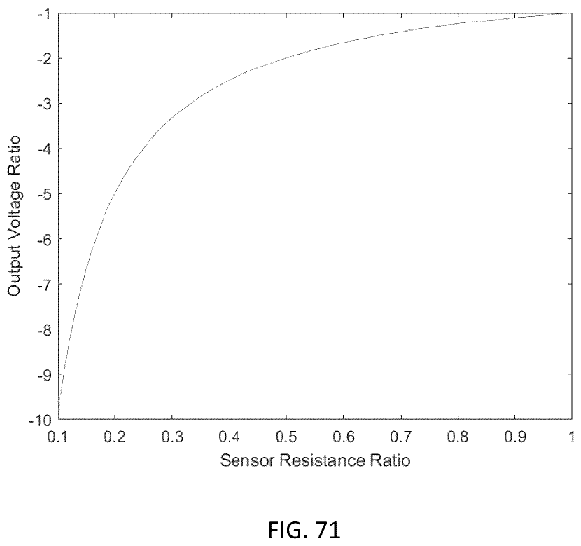

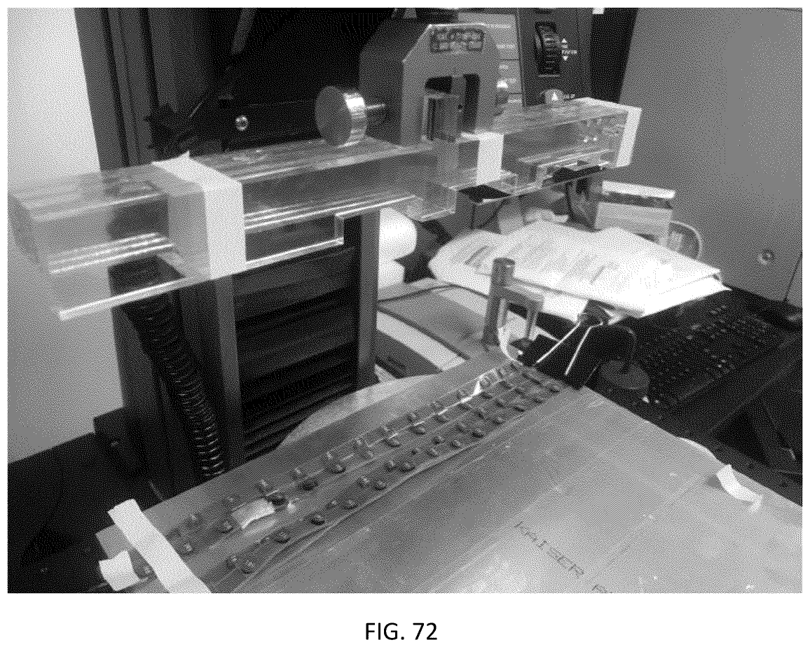

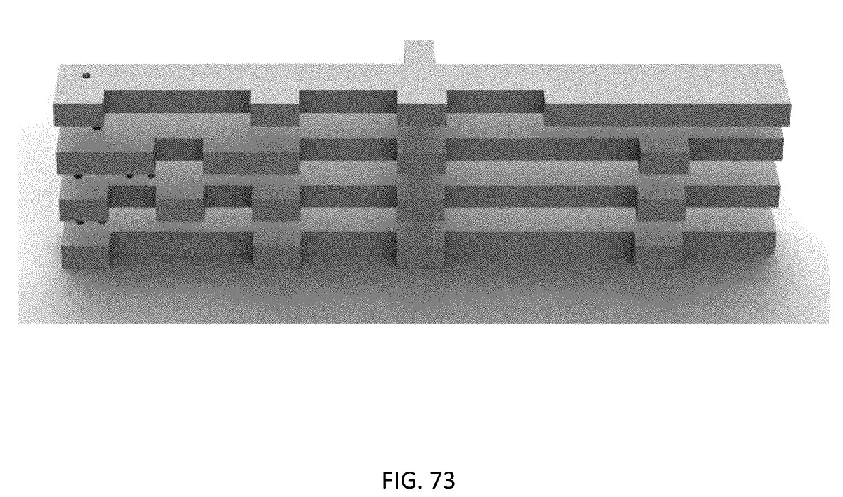

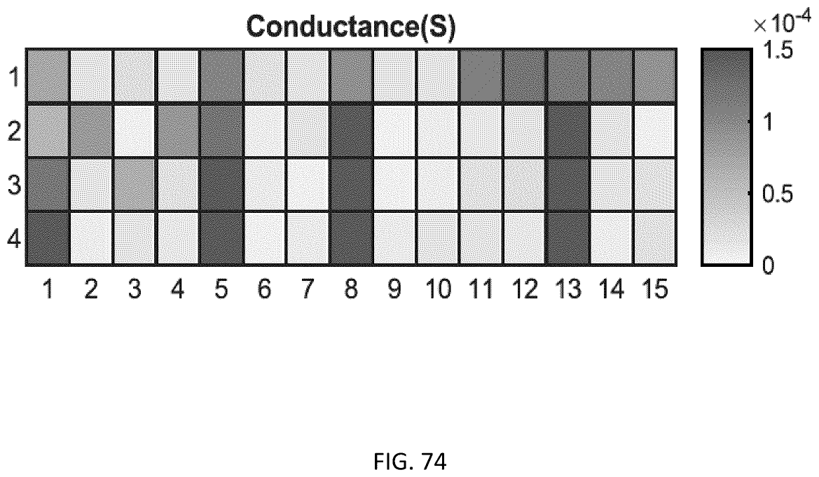

The performance of the system was evaluated by a variable-resistor array and by a physical test pattern pressed against the sensing elements (FIGS. 65-70), which was used to calculate the value of the sensing element resistance from voltage readings (see FIG. 71). A long belt of sensors was placed into a serpent-like shape under the “MIT” pattern (FIG. 72). As seen in FIG. 73, the test pattern consists of 4 presser plates that form the letters “MIT” when stacked. Such a test pattern causes alternating high-pressure and low-pressure points on the sensor, showing the MIT letters on the pressure map, but also highlighting the absence of cross-talk between sensing elements as observed in both evaluations (FIG. 74).

### Bolus Supporting Structure Design

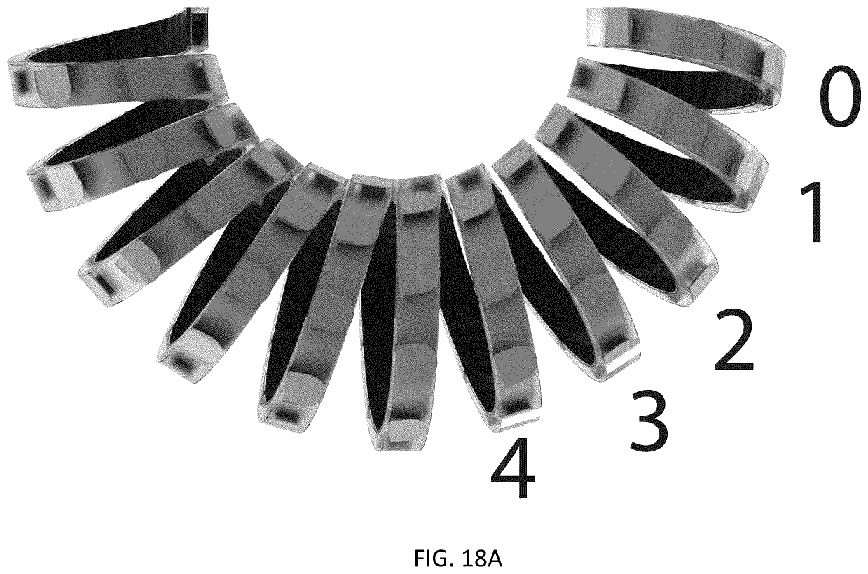

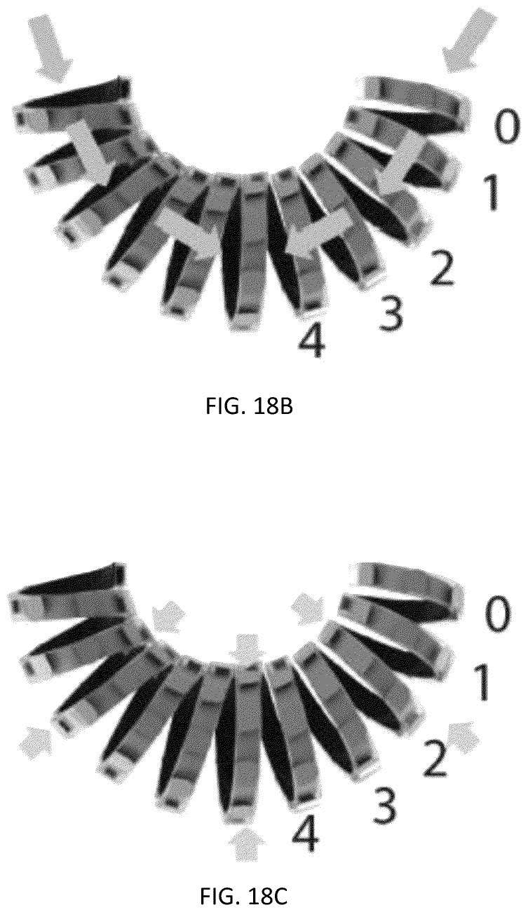

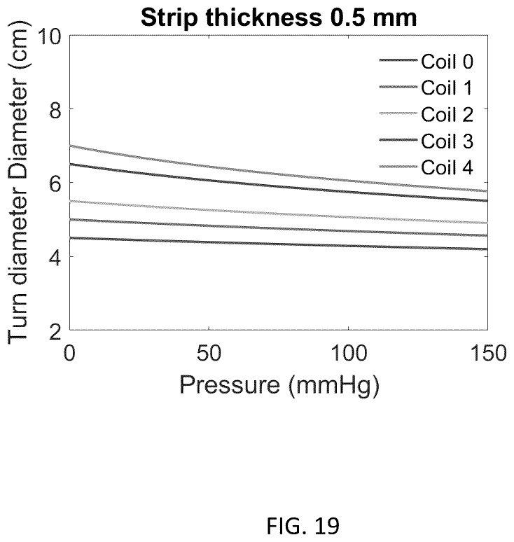

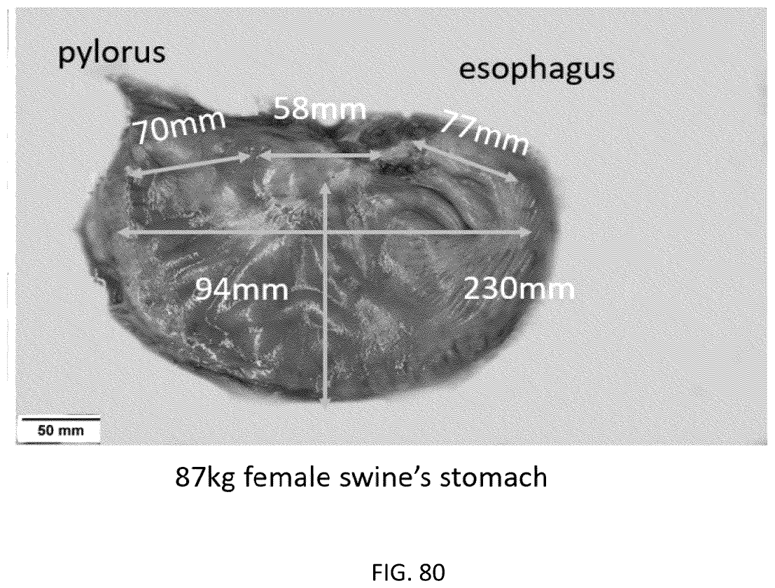

As mentioned above, the probes use the shape memory alloy Nitinol as the supporting structure material, allowing the probe to be a self-aligning in the GI tract. The Nitinol strips used for the esophagus and rectum are 1 mm thick, 54 cm wide; and 1 mm thick, 12 cm long. Using the dimensions measured from a swine stomach (FIG. 80), it was found that, to have a 2 cm resolution of pressure mapping, there should be approximately 90 pressure sensors. Thus, a slinky-like structure was used, which was 180 cm long. (see e.g., catheter 104 in FIG. 1) The challenge of building a supporting structure is to achieve a sufficiently large axial stiffness, while minimizing the thickness. In this way, the probe has a radial stiffness sufficiently small to be flexible and adaptable to the stomach, while being stiff enough to expand against its weight in the stomach. As shown in FIG. 18B, axial stiffness is the structure's ability to expand inside stomach against its weight. The probe is designed to be stiff enough axially to expand across the stomach. As shown in FIG. 18C, radial stiffness is the structure's ability to push against the surrounding stomach tissue. The probe's radial stiffness is designed to maintain sufficient flexibility so the probe can adapt to subjects with different stomach shapes/sizes.

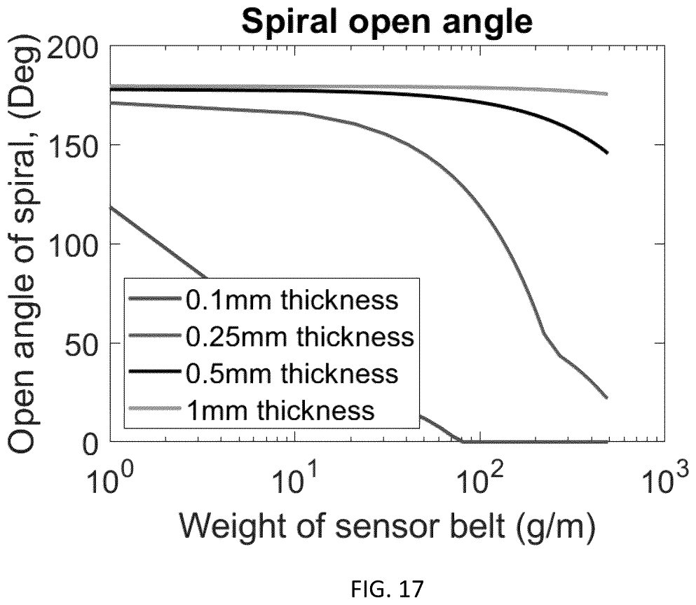

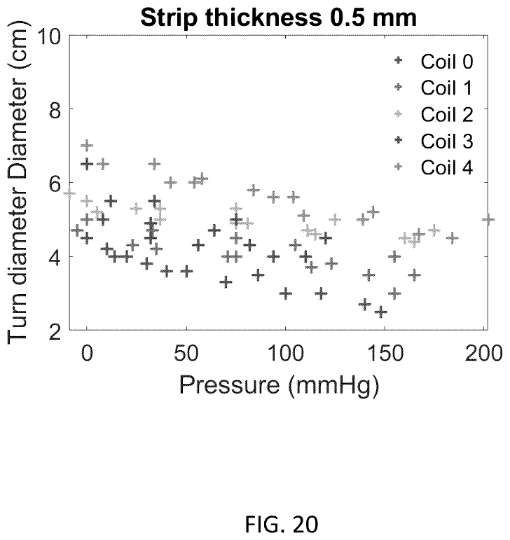

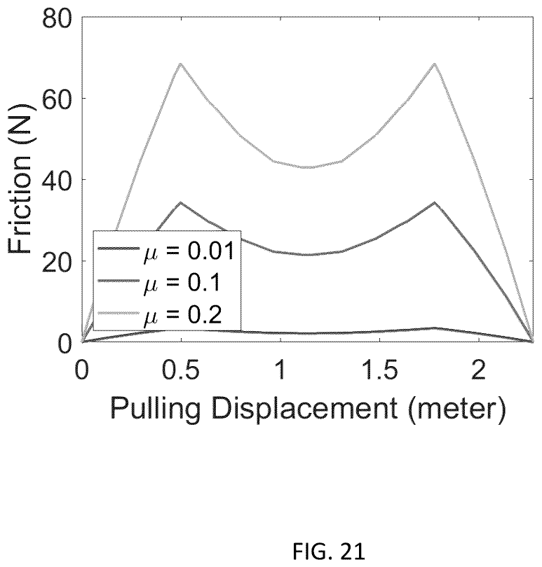

An axial stiffness model was built to study the influence of Nitinol thickness. The Nitinol sheets with a transition temperature of 35° C. with thicknesses of 0.1, 0.25, 0.5, and 1 mm were selected. The width of the strip was set as 1 cm considering the diameter of the overtube of 1.5 cm. As seen in FIG. 17, when the device has a weight of 100 g/m, a 0.1 mm thick device fully collapses, a 0.25 mm thick device has a 100 deg open angle, a 0.5 mm thick device has a 175 degree open angle, and a 1 mm thick version keeps an open angle of 179 degrees. 0.5 mm was found to be an optimal thickness since it allows a large open angle (expansion across the stomach) with the smallest thickness. In the study of radial stiffness, the diameter of each turn (see e.g., FIG. 18A) is a function of the pressure uniformly applied on the coil, and the result is shown in FIG. 19 In FIG. 20 each cross refers to a data point obtained from the radial stiffness testing experiment. In the experimental data, one can see from 0 to 150 mmHg, each turn is able to reduce the diameter by approximately 2 cm. Considering the diameter of a 75-100 kg swine's stomach is 4-7 cm, the supporting structure is sufficiently flexible and adaptable for intended application. For example, in the swine model, the supporting structure is able to reduce total diameter by 50-30% when the maximum possible pressure is applied.

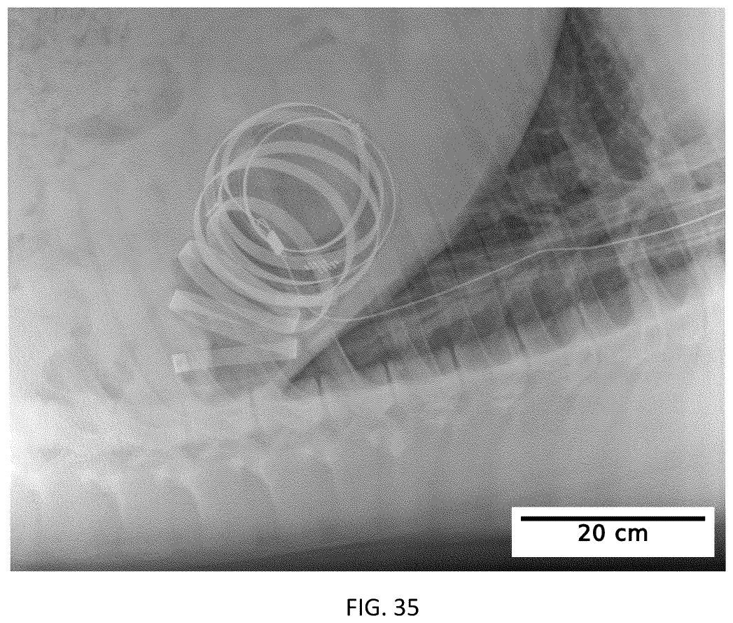

The supporting structure with 0.5 mm thickness is then fabricated and integrated with the sensor belt. The probe is stiff but flexible, which allows it to be stretched, or compressed for adaptation purposes. The probe was deployed into animals with different body weights. As shown in FIG. 35, the supporting structure can automatically align by itself by adapting the shape of the stomach.

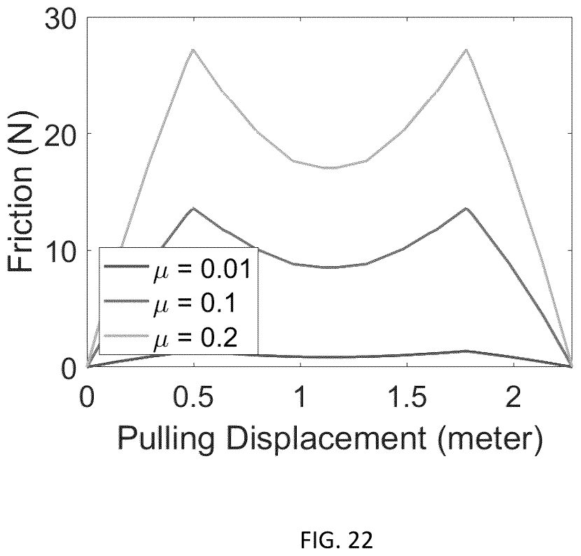

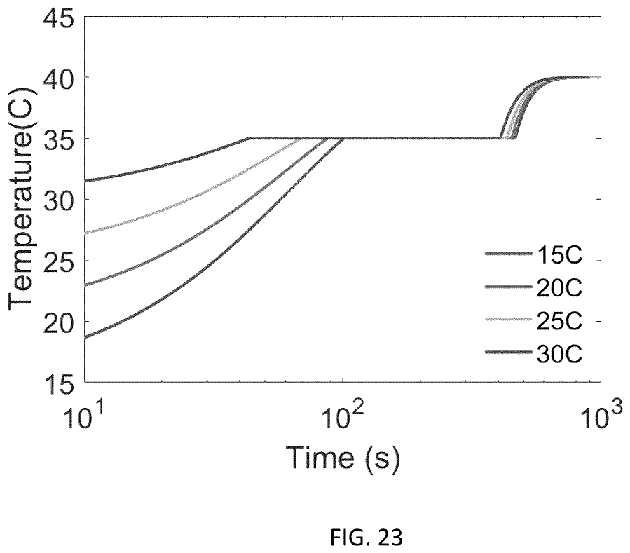

Correspondingly, the friction forces during the deploying and retrieval processes were also investigated. FIGS. 21 and 22 show the friction on the supporting structure in the overtube as a function of how far it has been pulled/pushed out/into the overtube. FIG. 22 shows the friction when the supporting structure is in the austenite phase (above transition temperature, during retrieval), and FIG. 21 shows the friction when the structure is in the martensite phase (below transition temperature, during insertion). In the austenite phase, the maximum friction reaches 70N with a friction coefficient of 0.2. With the same friction coefficient, in the martensite phase, the friction has a maximum value of 27 N with a friction coefficient of 0.2. In both cases, the maximum friction is proportional to the friction coefficient. Accordingly, to minimize friction, the probes should be well lubricated during both the deploying and the retrieval processes. In the in vivo experiment, an edible spray oil can (PAM® Cooking Spray) was used to create an oil-rich environment in the overtube.

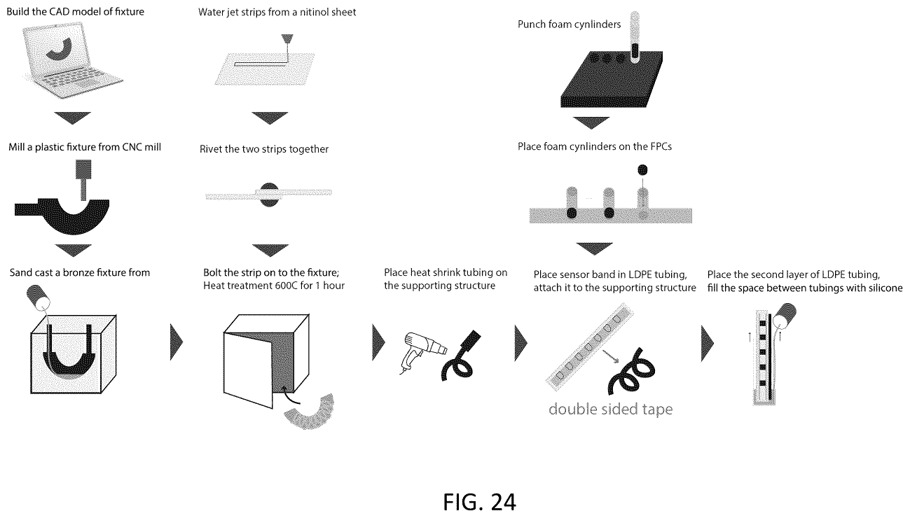

Additionally, during the deploying process, lubrication can help to reduce the time for insertion, thereby ensuring that the phase transition of the Nitinol from the martensite to austenite phase will not finish until the probe reaches the stomach. Accordingly, a heat transfer model was developed to study the temperature of the device as a function of the time in the body of the swine (FIG. 23). The plateau in the graph shown the period when the device reaches the transition temperature. The heat is absorbed into the material as latent heat to drive the phase transition. Before the plateau, the device's temperature rises from the room temperature, 15, 20, 25, and 30° C. respectively. To minimize the friction and prevent the phase transition from occurring in the overtube, the probe should reach the stomach before tₚ, when the phase transition starts. Given the length of the overtube is 0.5 m, total delivery time with different room temperatures are shown in Table 1.

TABLE 1
Maximum time to prevent in-overtube phase
transition in the in vivo experiments.

| Room temperature | | Maximum allowed |
|---|---|---|
| (Celsius) | tₚ (s) | delivery time (min) |
| 15 | 100 | 4.7 |
| 20 | 90 | 4.2 |
| 25 | 85 | 4 |
| 30 | 40 | 2 |

Sensor Probe Prototyping and Integration with Supporting Structure

Each of the probes has a linear pressure sensor system composed of a flexible printed circuit (FPC), a nitinol strip as the supporting structure, and two layers of sealing for impermeability (FIG. 2). The probes require no microfabrication nor clean room process, although such processes may be used. As discussed above, to design the conformal GI tract motility mapping probes with piezoresistive sensors, materials with low elastic modulus and high electrical conductivity are desirable. The foam's electrical resistance decreases when the pressure is applied uniaxially onto it. To understand the mechanism of this phenomenon, the microstructure and electromechanical characteristics of the foams was characterized. As can be seen from the scanning electron microscopy (SEM) images in FIG. 6, all four foams exhibit a porous structure, with the pore diameter of 0.5-1 mm. The 3D morphology was evaluated using a 3D model from CT scan imaging data with 3 μm resolution (FIG. 54). All foams had a porosity of over 90% (FIG. 55). In the neutral state, where there is no pressure applied on the foam, the electrons flow through the skeleton of the foam. Once the foam experiences stress, the porous structure collapses and contacting points are created inside the foam. With a larger stress, more contacting points are created, thus the electrical resistance of the foam decreases. The conductivity of the sample 16-1231 was superior to the other foams (FIG. 7). Sample 16-1231 had a conductivity of 10 S/cm, while other foams have significantly smaller conductivity. The foam 37704-ND had the highest Young's modulus value (E=754±24 kPa), while the softer foam (16-1231-ND) had a value of E=180±22 kPa (FIG. 9). In this application, a desirable value for E is ~100 kPa because with a motility compression pressure ~40 kPa the material will have a significant deformation and thus a change of electrical resistance. The desired conductivity of the material is ~10 S/m. With this conductivity, the measured current would be ~1 mA because ~10 V of bias voltage is applied onto the sensing material. This amount of current is large enough for an accurate electrical resistance scanning (the resolution of current measurement is ~1 uA in our system) while maintaining the voltage below the dangerous threshold of 30V(36).

Based on our results, we concluded that the best candidate was the sample 16-1231-ND, with its low Young's modulus E (FIG. 9) and high electrical conductivity (FIG. 7). The piezoresistive behavior of the foam 16-1231-ND exhibited good linearity up to 50% deformation, with a gauge factor (GF) value of 1.0±0.4 (FIG. 10). A twin spring model was developed, as described below, to optimize the geometric parameters of the sensor cell, where the foam is modeled as a linear spring connected in parallel with the FPC modeled as a U-turn spring. The model is verified by observing the mechanical behavior of the sensor cell (FIG. 53). When the sample is compressed, the foam cell shrinks in height and behaves like a linear spring, and the diameter of the U turn gets smaller. The sensor is applied to 18 cycles of compression to verify its response linearity and further 1000 cycles to confirm its durability. (See FIGS. 8 and 57) The experimental data are in accordance with the twin spring model prediction.

### In Vivo Validation of Motility Mapping Probe on a Swine Model

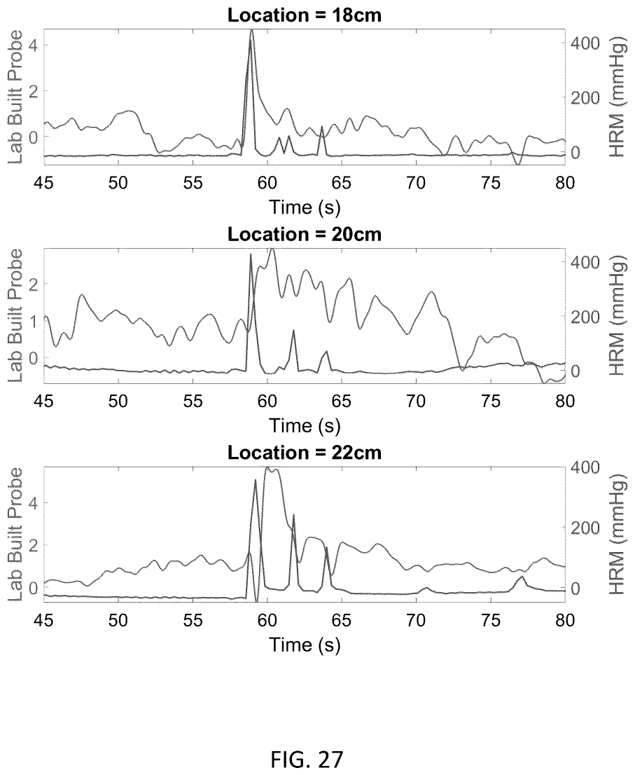

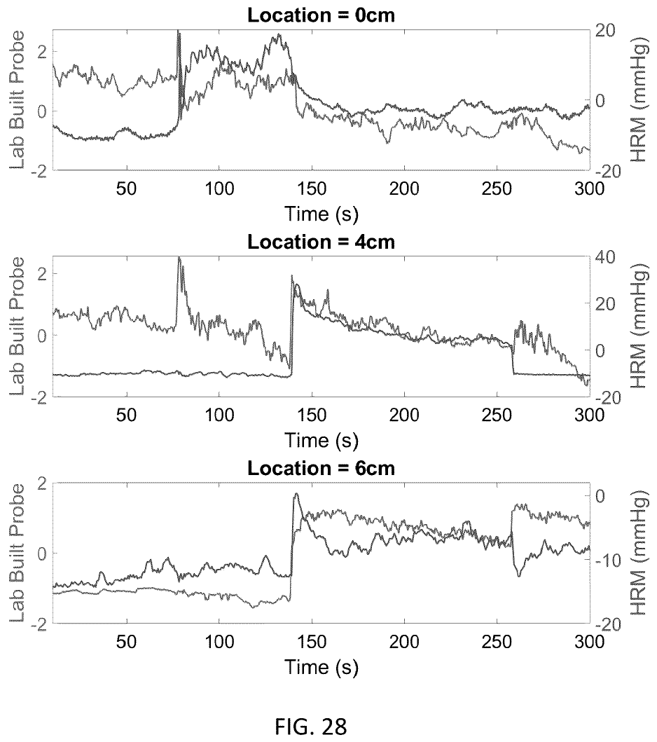

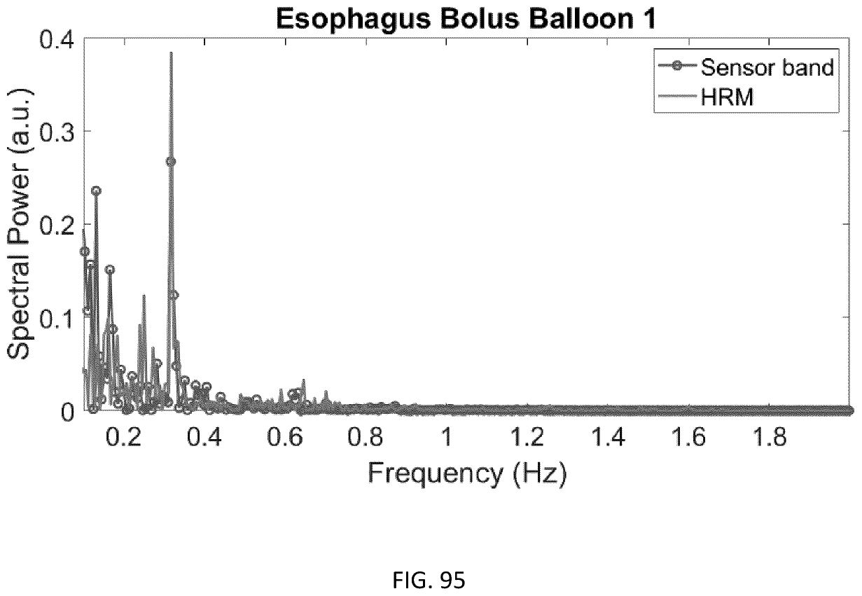

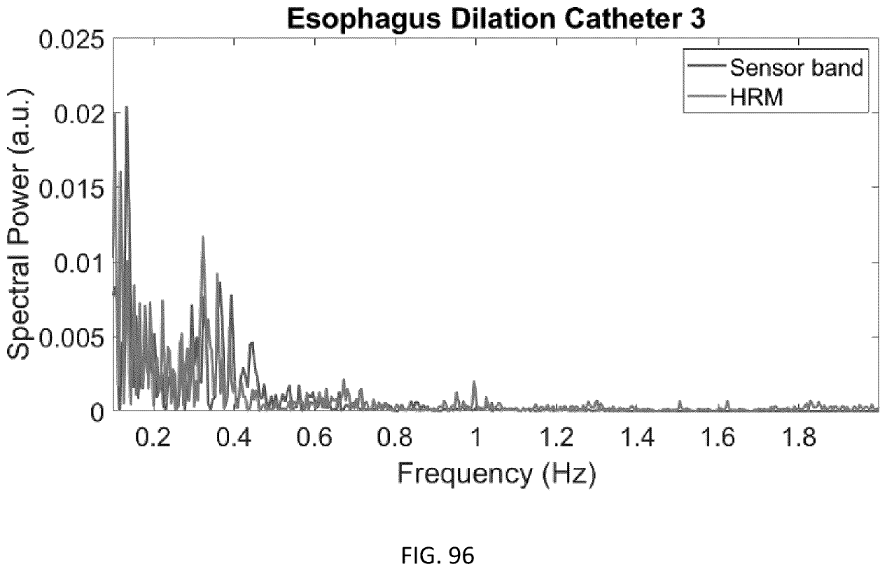

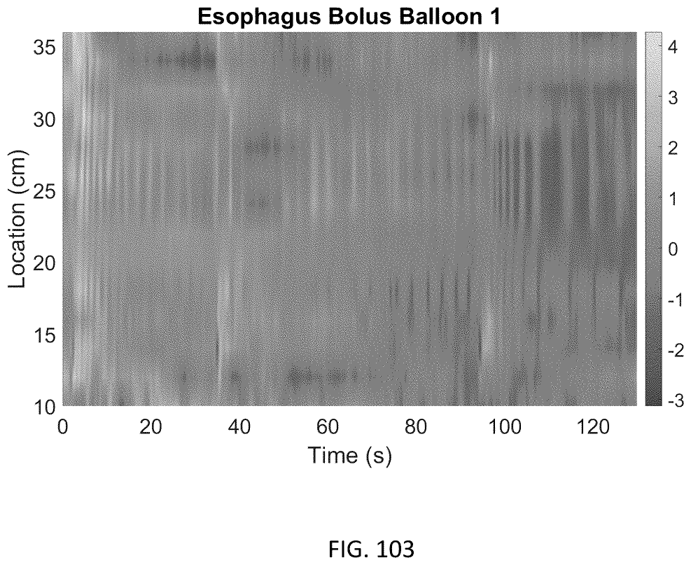

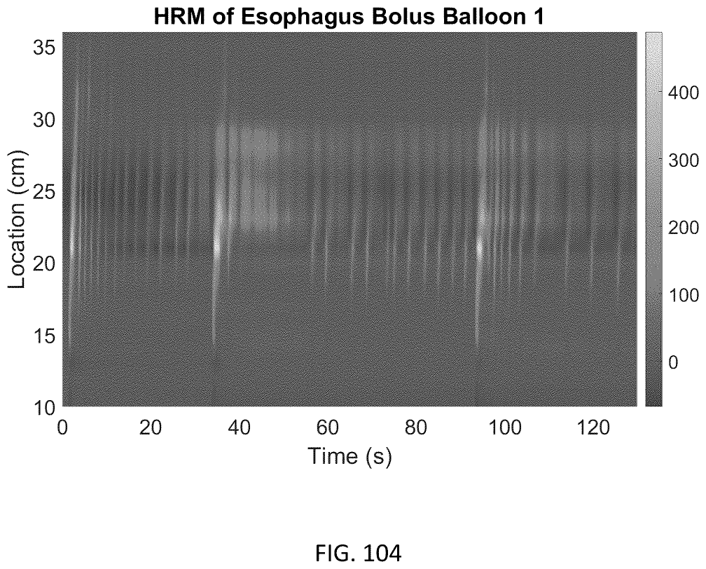

A Yorkshire female swine with body weights of 75-100 kg was used as the large mammal model to validate the platform. A clinical HRM was used as the tool of calibration and comparison. The swine was sedated before the experiment and several stimulation methods were used to generate motility/reflex. A bolus balloon was first used to stimulate the peristalsis in the esophagus. As can be seen in FIG. 26, three points of 18, 20, and 22 cm from the mouth of the swine were quantitatively compared. The peristalsis peaks were captured by both tubular sensing probe and HRM, with correlation factors of 0.581, 0.486, and 0.487. The p-values of the Wilcoxon rank sum test are 4E-17, 5E-8, and 2E-8, respectively. The spectral intensity graph of the two sensing readings is shown in FIG. 95. The correlation between the two spectra is 0.6645. The pressure-time-location graphs from 10 to 36 cm are shown in FIGS. 103 and 104. A relationship can be seen in the two graphs, where three strong pulses are observed at t=0, 35, and 90 s with following periodic contractions. A dilation catheter was then used to stimulate a single pulse of contraction in the esophagus. FIG. 27 shows the strong single pulse captured by both devices, with correlation factors of 0.5578, 0.5597, and 0.6120 at 18, 20, 22 cm sensing points, and the p-values of the Wilcoxon rank sum test are 5E-6, 1E-5, and 2E-6 in this case study. The spectral intensity graph of the two devices is shown in FIG. 96, with a correlation factor 0.6202 for the two signals.

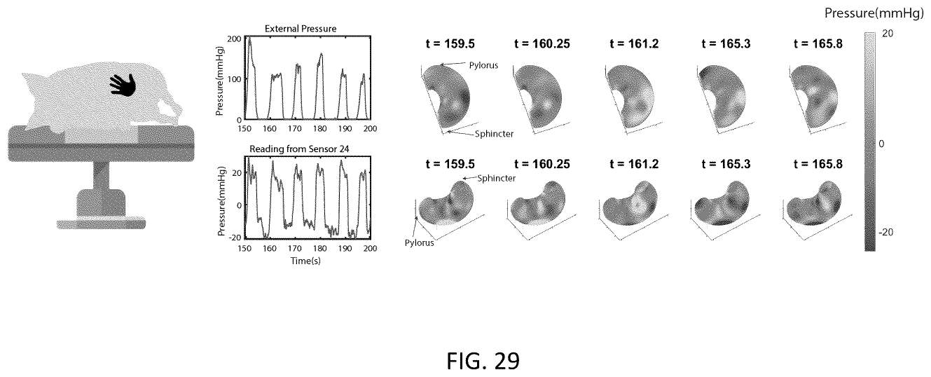

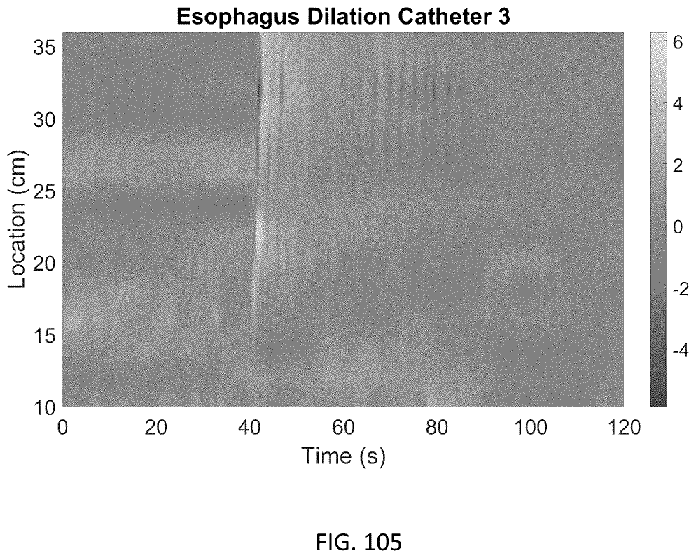

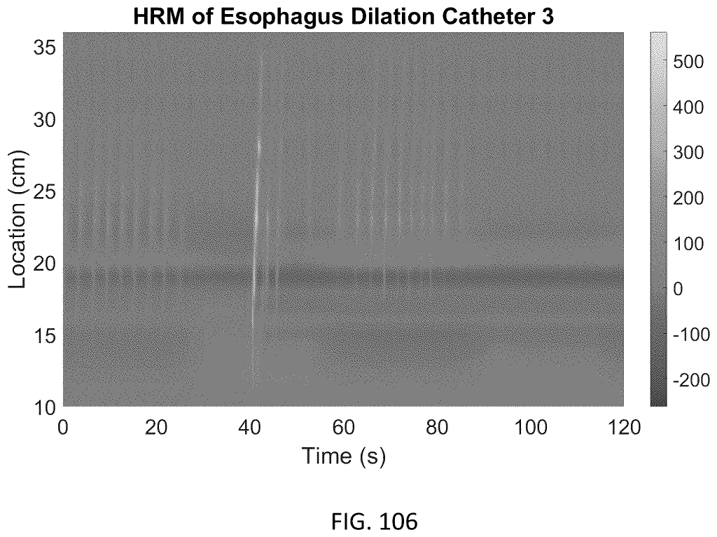

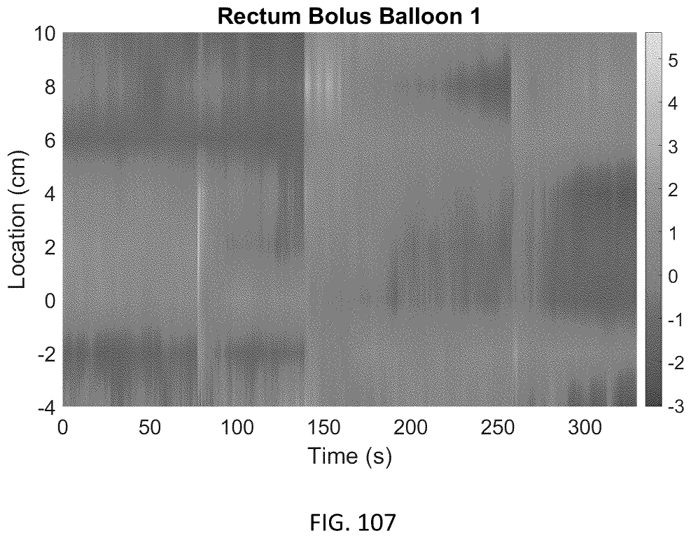

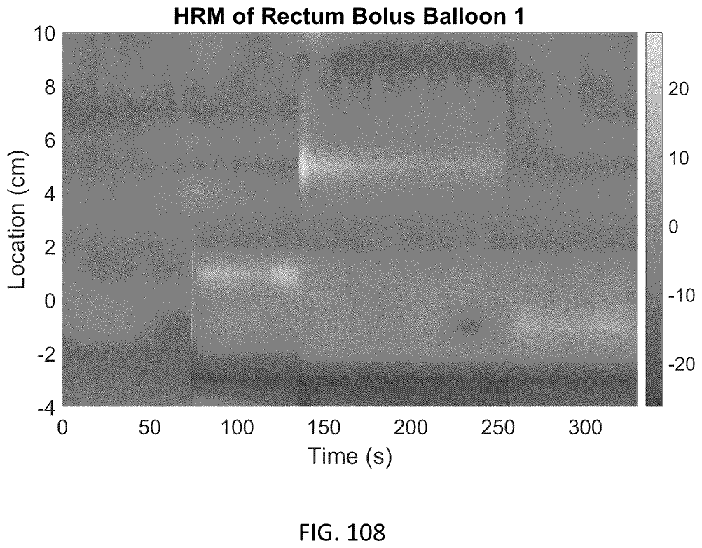

The pressure-time-location graphs of the dilation catheter case are shown in FIGS. 105 and 106. A single strong pulse was recorded in both devices, followed by the subsequent smaller pulses. A bolus balloon was placed into the rectum of the swine along with the sensing probe and the HRM catheter (FIG. 28) to evaluate an endogenous reflex in the rectum called the rectoanal inhibitory reflex, which involves relaxation of the internal anal sphincter in response to rectal distention to facilitate the passage of stool. We placed a balloon into the rectum of the swine along with the sensing probe and the HRM catheter. We inserted the balloon at 80s (see FIG. 27) and thus at the anus there was a significant increase of pressure at around that time. This insertion was also captured by the sensing probe at location=4 cm shown in the blue line. At 140 seconds, the bolus balloon was inflated. From the two pressure sensors in the rectum, a pressure increase was observed. From the anus pressure sensors, a gradual decrease of the pressure was observed due to the rectoanal inhibitory reflex, where the anus is forced to relax involuntarily due to the expansion of rectum. The data from the rectum pressure mapping is shown in the pressure-time-location graphs of FIGS. 107 and 108. The inflation event can be observed with an increase of pressure at locations of 4 cm and 6 cm at 140 seconds, and the relaxation event in the anus at location of 0 cm occurs at about the same time point.

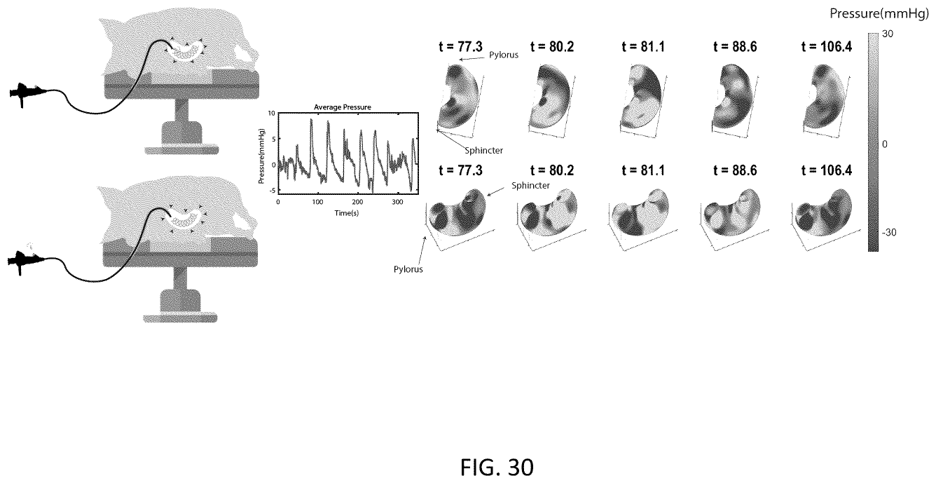

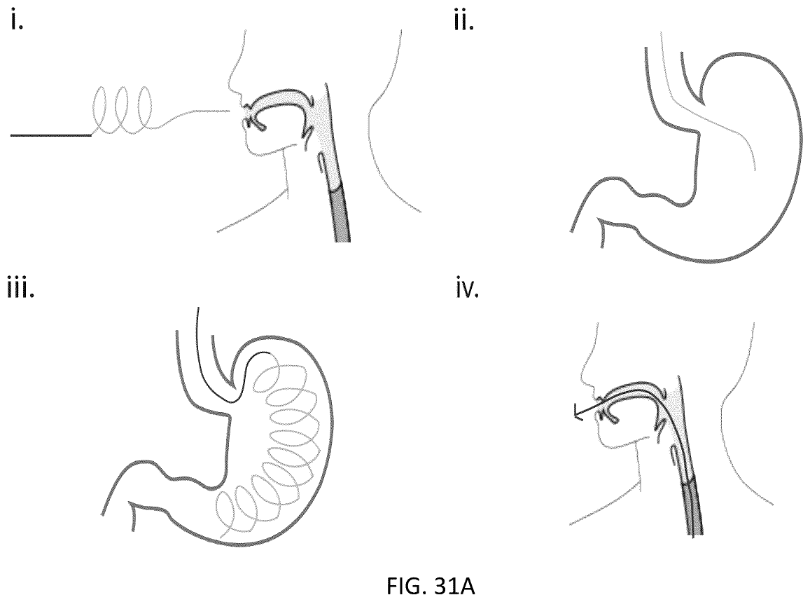

To test the ability of the stomach sensing probe to map the pressure distribution in vivo, palpation was applied by hand onto the abdomen of the swine. A force sensor was placed under the hand to record the pressure applied from the hand. Pressure sensor #24 on the probe was picked in comparison with the external pressure. As shown in FIG. 29, the timings of the pressure peaks on the pressure sensor #24 match with the reading from the external force sensor. The pressure distribution of the device as a function of time is shown as well. As can be seen, the external compression causes pressure changes in an area on the small curvature close to the pylorus, and an area on the bigger curvature close to the sphincter. An endoscope was then used to inflate and deflate the stomach with a period around 30 s (FIG. 30). In the average pressure curves, each peak relates to a deflation event. The pressure distribution on the device shows a large area of pressure increase when the deflation occurs.

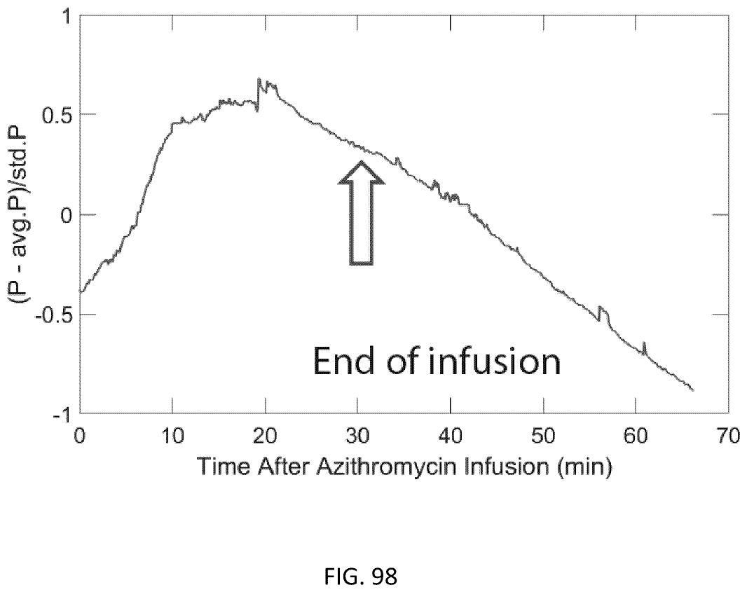

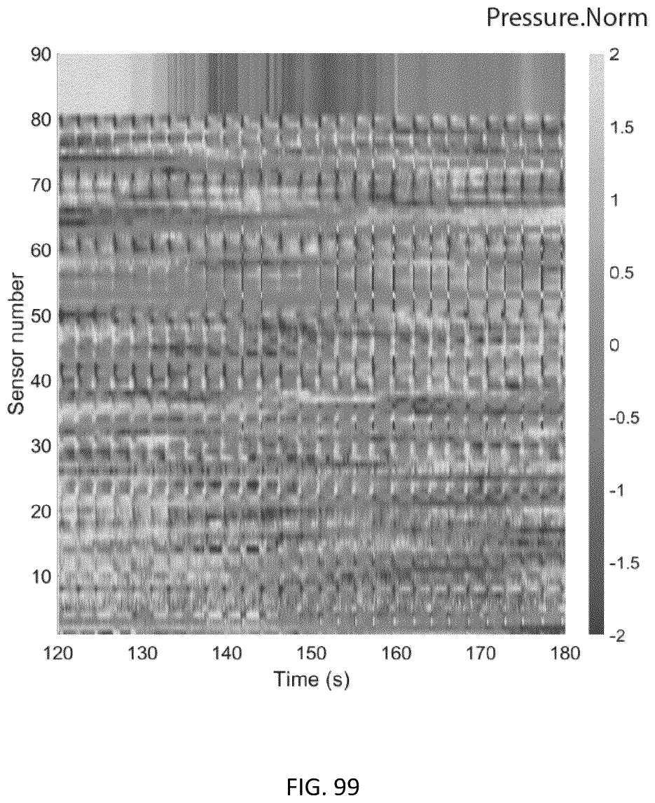

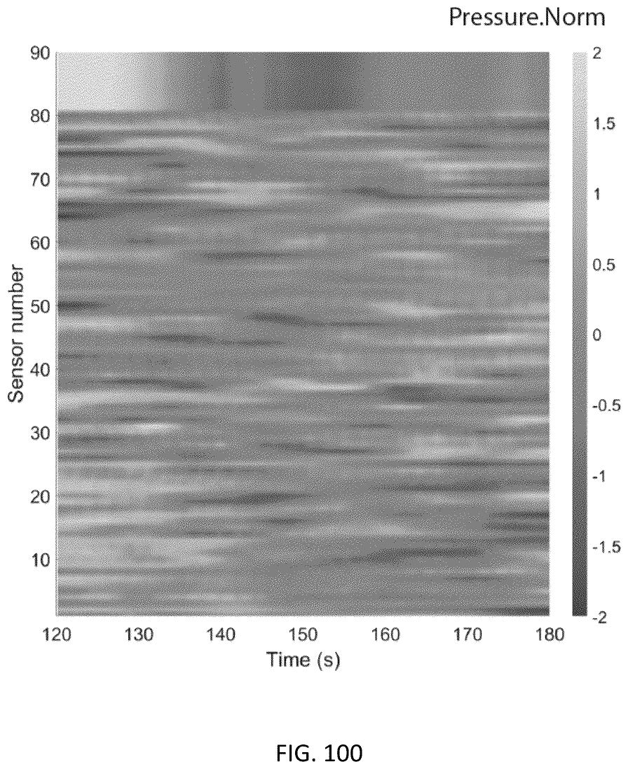

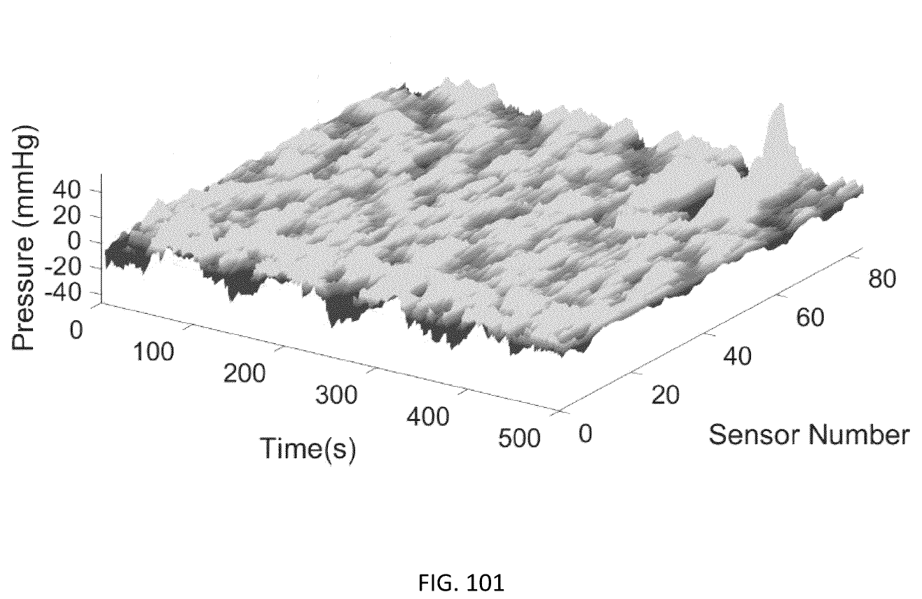

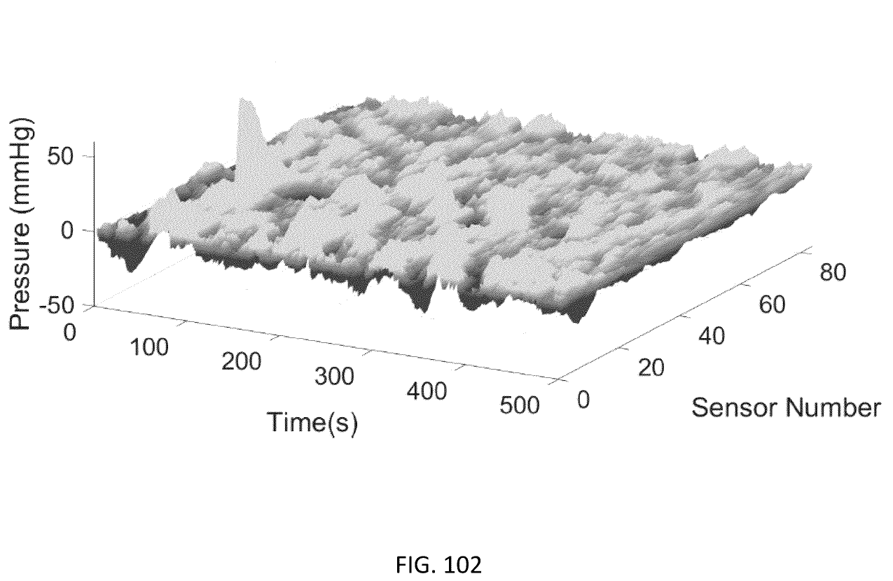

When the sensing probe was in the stomach of a pig, 500 mg Azithromycin (USP) into was infused into the swine for 30 mins using a syringe pump. The average pressure reading from the stomach sensing probe is shown in FIG. 98. The increase of pressure was observed in the first 20 mins of infusion and a relaxation afterward. The readings of the 90 sensors are shown in FIG. 99 as raw data and in FIG. 100 after applying a moving average filter. In FIGS. 95 and 96 a respiration pattern was observed in the recording. In FIGS. 101 and 102, the height plot of the data from FIGS. 99 and 100, respectively, and the 3D interface of the pressure distribution shows a migration pattern.

### Methods

Three-dimensional (3D) models of the platform were designed in Solidworks (Dassault Systemes, Velizy-Villacoublay, France). The manufacturing G-code was generated from Fusion 360 (Autodesk, San Rafael, USA). The Tormach 440 PCNC (Tormach LLC, Waunakee, USA) was used to mill a plastic fixture. The raw material of the fixture was purchased from McMaster-carr (McMaster-carr, Elmhurst, USA). The circuits were designed on Eagle PCB (Autodesk, San Rafael, USA) and manufactured by Bittele Electronics (Markham, Canada). Other relevant electronic components were purchased from Digi-Key Electronics (Thief River Falls, USA). LDPE tubing were purchased from Uline (Pleasant Prairie, USA). Eco-flex silicone was purchased from Smooth-On (Macungie, USA). Tetrahydrofuran was purchased from Sigma-Aldrich (St. Louis, USA). Styrene-ethylene-butylene-styrene polymer was supplied by Kraton (Houston, USA). Multiwall carbon nanotubes were purchased from US Research Nanomaterials (Houston, USA). The Nitinol alloy was purchased from Kellogg's Research Labs (Salem, USA) and water jetting was done on an OMAX water jetting machine (Kent, USA). Female swine used for in vivo studies were acquired from Tufts University (Grafton, USA). The data visualization and processing were performed on MATLAB (Matlab, Natick, USA). The Universal Testing Machines was purchased from Instron (Instron, Norwood, USA).

### Fabrication and Testing of Three Pressure-Sensitive Resistive Polymers

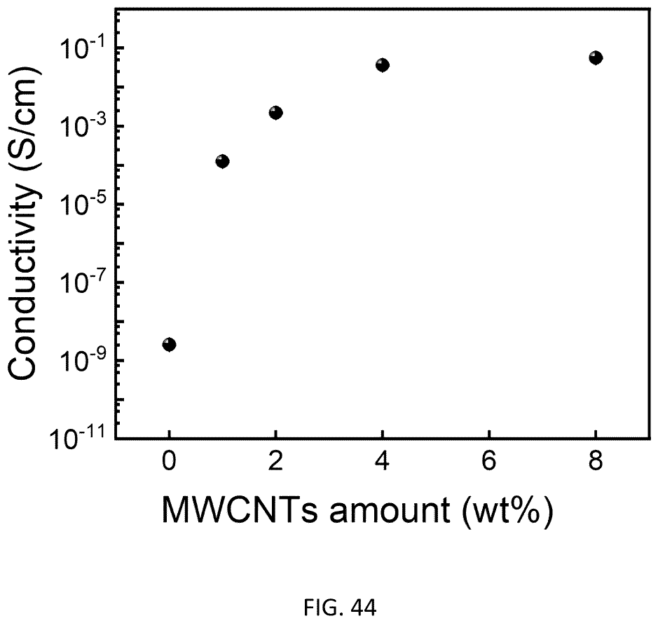

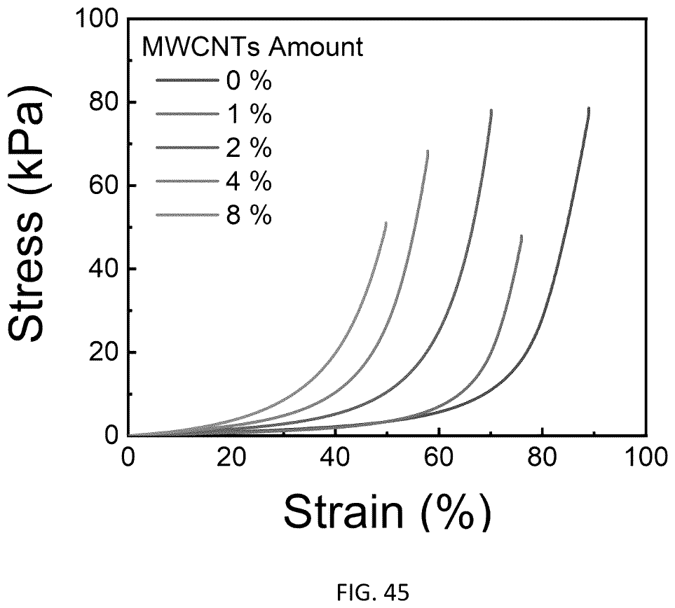

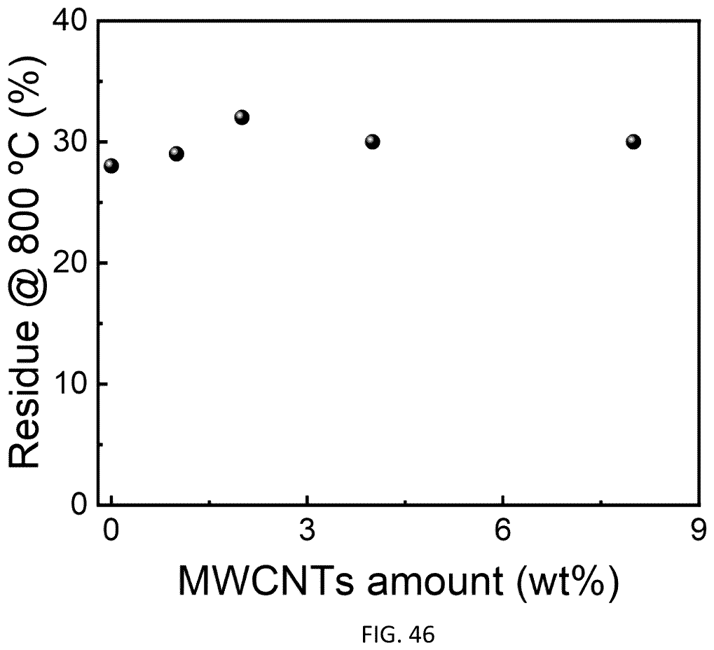

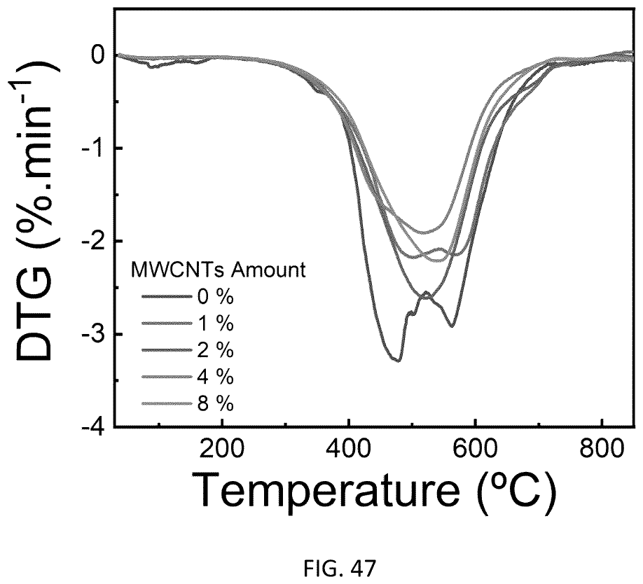

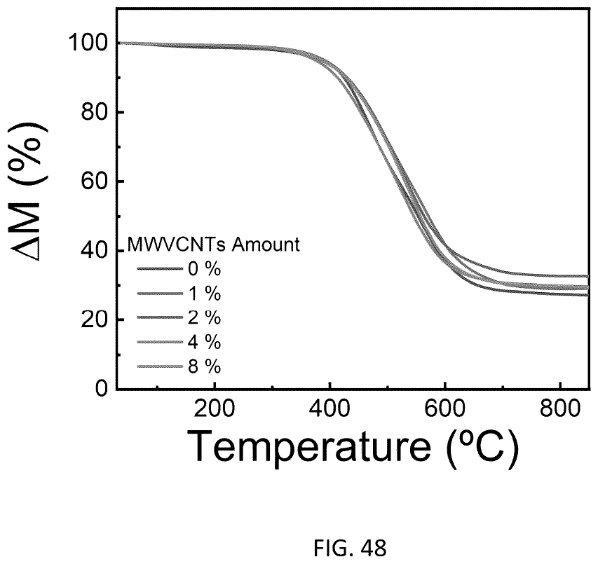

Eco-flex 00-30 was used as the base material for porous pressure-sensitive foam. Approximately 2.5 g of Part A Eco-flex, 2.5 g of Part B Eco-flex, and different amounts of MWCNTs were then added (0-8 wt %) and manually stirred with a spatula. After, 25 g of NaCl particles were added to the previous blend, stirred manually, and cast the mixture on a glass petri dish and left it to cure overnight. The cured polymer was placed in a DI water bath for 3 days and then left in a fume hood to dry at room temperature for 2 days. The final product is shown in FIG. 57. The foam has formed a porous structure with a conductivity that was dependent on the amount of conductive filler added to the elastomeric matrix. A maximum of 5.6 S/m was achieved for the sample with 8 wt % MWCNTs (FIG. 44). It was observed that increasing the amount of conductive filler led to an increase on the mechanical stiffness of the Eco-flex foams (FIG. 45). Furthermore, the prepared nanocomposites are thermally stable up to 300° C., followed by decrease in the sample weight that is due to the volatilization of the Eco-Flex matrix (FIG. 48). It was found for materials doped with 0-8 wt % MWCNTs, that they all had 28±3% residue at 800° C. (FIG. 46), suggesting that the MWCNTs could chemically bond to the polymer chains during the elastomer curing. FIG. 47 shows the rate of weight loss during the thermal weight loss experiment.

To adhere the foams onto FPC, double sided conductive tape was used on 16-1231 foam, which showed good adhesion on FPC boards.

### Single Sensor Cell Twin Spring Model

To optimize the design of a single sensor cell, a twin spring model is developed in the work. As can be seen from FIGS. 51-53, when an external force is applied onto the sensor cell, the foam and the U turn both resist the compression. When an external force is applied to the sensor cell, the foam and the U turn both resist compression. Thus, they be modeled as two springs connected in parallel. Assuming the foam is a homogenous material with a constant cross-sectional area, its spring constant can be calculated as a continuous material. Correspondingly, the foam (e.g., the compressible member 140) has an equivalent spring constant (k_{f}) defined by Equation 2, where E_{f} is the Young's modulus of the foam:

$${k}_{f} = \frac{{E}_{f} {A}_{f}}{{h}_{f}} \qquad Equation 2$$

Correspondingly, the u-shaped portion of the circuit board 110 can be modeled as a flat U-turn spring model, whose equivalent spring constant k_{fpc} is defined by Equations 3 and 4, where E_{fpc} and μ are Young's modulus and Poisson Ratio of the FPC material, respectively:

$${k}_{fpc} = \frac{8 ( B )}{{h}^{3}} \qquad Equation 3$$

$$B = \frac{{wt}^{3} {E}_{fpc}}{12 ( 1 - {v}^{2} )} \qquad Equation 4$$

The force F applied on the sensor is related to the pressure the sensor experiences and the area where the pressure is applied. When the sensor is compressed to have a smaller height h, more area is delivered under the compressor. This is illustrated at the left panel of FIG. 51. Thus, the force F applied to the pressure sensor 130 is related to the pressure acting on the pressure sensor 130 as shown by Equation 5:

$$F = P ( A + \frac{w}{2} ( L - \frac{π h}{2} ) ) \qquad Equation 5$$

In Equation 5, P is the pressure, A is the area of the FPC top pad, W is the width of the FPC, (L−πh/2)/2 is the length of the U-turn in FIG. 51 on side of facing the pressure. Thus, for a given height h, the increase of force F and pressure P can be written as Equation 6. In the twin spring model, the total spring constant of the sensor is expressed as Equation 7 considering the foam and the U-turn are working in parallel. By taking the derivative of Fin respect to time k, we obtain Equation 8 where the height h and pressure P are related. Thus, the derivative equation of the sensor height (h) and pressure (P) can be obtained as follows:

$$d F = dP ( A + \frac{w}{2} ( L - \frac{π h}{2} ) ) \qquad Equation 6$$

$$k = {k}_{f} + {k}_{fpc} \qquad Equation 7$$

$$- dh = \frac{dF}{k} = \frac{dP ( A + \frac{w}{2} ( L - \frac{π h}{2} ) )}{\frac{8 B}{{h}^{3}} + {k}_{f}} \qquad Equation 8$$

Taking the integration of Equation 8 and applying the initial condition, the relation of height h and pressure P is expressed as follows, where h0 is the height of the sensor in an uncompressed state (i.e., with no pressure being applied to the sensor) and CST is an integration constant:

$$- \frac{( a {c}^{3} + {b}^{3} d ) log ( b - c h )}{{b}^{3} c} - \frac{a ( b + 2 c h )}{2 {b}^{2} {h}^{2}} + \frac{a {c}^{3} log ( h )}{{b}^{3}} = - P + CST \qquad Equation 9$$

$$P = ( \frac{a {c}^{3}}{{b}^{3}} + \frac{d}{c} ) log ( \frac{b - c h}{b - c {h}_{0}} ) + \frac{a ( b + c h )}{2 {b}^{2} {h}^{2}} - \frac{a ( b + 2 c {h}_{0} )}{2 {b}^{2} {h}_{0}^{2}} - \frac{a {c}^{3}}{{b}^{3}} log ( \frac{h}{{h}_{0}} ) \qquad Equation 10$$

The constants are:

$$a = 8 B$$

$$b = A + \frac{w L}{2}$$

$$c = \frac{π w}{4}$$

$$d = {k}_{f}$$

### Bolus Supporting Structure Mechanical Model

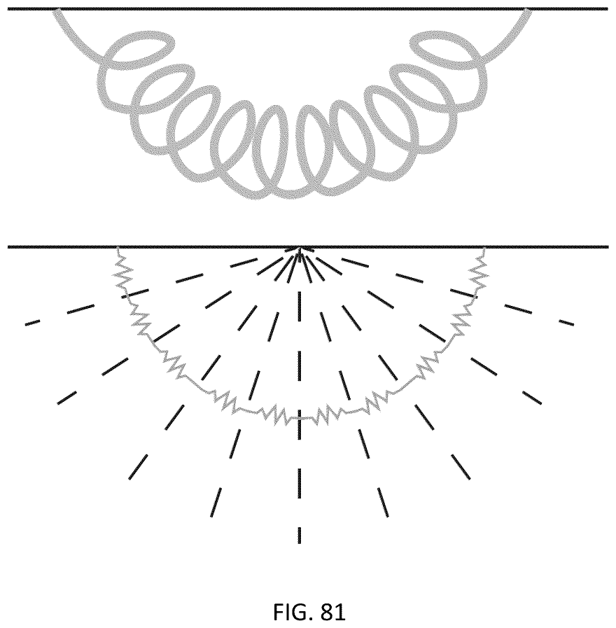

A mechanical model to describe the axial stiffness of the supporting structure is developed. Each turn in the coil is modeled as a coil spring, which will resist the weight of the sensor system above it, as can be seen from FIG. 81. The spring constant (kᵢ) of each turn is described by Equation 11, where β=0.4, E is the Modulus of Elasticity of the coil, b is the width of the spring strip that forms the coil, and D is the diameter of the coil:

$${k}_{i} = \frac{β {Ebt}^{3}}{{D}^{3}} \qquad Equation 11$$

The change of length of each turn due to the weight of sensor system is δₙ:

$${d}_{n} = \frac{{\sum }_{i}^{1} {w}_{i} g cos {θ}_{i}}{{k}_{n}} \qquad Equation 12$$

$${d}_{n} < {d}_{n}^{max} = \frac{{\sum }_{i}^{1} {w}_{i} g}{{k}_{n}} \qquad Equation 13$$

Since each turn inside the coil structure works as a helical spring, it needs to support the structure and fight against the weights of every other turns above itself. The weight of each turn is wᵢ, and cos (θᵢ) is the projection of gravity onto the axis of each turn, where θᵢ is the angle between plane of the turn relative to the gravity vector acting on the turn. Notice that in the in vivo environment, this angle is depending on the pose of the subject. Summing all the weights and dividing the axial spring constant kᵢ, the change of length of each turn n due to the weight of sensor system is δₙ. In an in vivo environment, the projection of the gravity vector onto the axis is dependent on the pose of the subject. To ensure the structure will have sufficient axial stiffness, the maximum possible compression length

$${δ}_{n}^{max}$$

was used in the calculation.

### Radial Stiffness Model of the Bolus Structure

A model to predict the radial stiffness of the bolus supporting structure is illustrated in FIG. 81. First by employing the principle of virtual work, a relationship is established between internal torque in a turn of the supporting structure and the external pressure, where M is the internal torque, P is the pressure applied on the structure (e.g., to the circumference of the coil), W is the width of turn studying, and D is the diameter of the turn, and r is the radius of the turn. When the coil turns over a small angle dθ caused by the circumference force, the work stored into the internal stress is Mdθ. This increase of energy is caused by the volume work done by the pressure P, which is PWπDdr. According to the principle of virtual work, the energy done by the volume work is the same as the energy that is stored in the coil. Further, the relationship between dθ and dr given the length of the coil does not change:

$$( 2 π + d θ ) ( r + d r ) = 2 π r \qquad Equation 14$$

$$d θ = \frac{- 2 π d r}{r} \qquad Equation 15$$

Here, we neglect dθdr in the derivation. Thus, a relationship can be established between the internal torque of a turn of the supporting structure and the external pressure from the principle of virtual work:

$$\sum d ( Work ) = M d θ + P W μ D d r = 0 \qquad Equation 16$$

$$M = \frac{{PWD}^{2}}{4} \qquad Equation 17$$

Correspondingly, each turn is modeled as a coil spring, where L is the total length of the coil spring, b is the width of the spring, t is the thickness of the spring (the FPC), E is the Young's modulus of the material, and θ is the angle the spring has turned over:

$$M = \frac{π {Ebt}^{3} θ}{6 L} \qquad Equation 18$$

The angle turned over due to external pressure can be expressed according to Equation 16, where Nₜ is the number of turns:

$$θ = 2 π ( {N}_{t} - 1 ) \qquad Equation 19$$

The total length of the turn is expressed as:

$$L = π {N}_{t} D \qquad Equation 20$$

Because total length of this turn is the same before and after it is experiencing pressure, where D₀ is the initial diameter:

$$π {N}_{t} D = {D}_{0} π \qquad Equation 21$$

Substituting the relevant expressions results in the following relationship between the diameter and the external pressure:

$$\frac{3 {PWD}_{0}^{3}}{4 π {Ebt}^{3}} ( \frac{D}{{D}_{0}} ) + ( \frac{D}{{D}_{0}} ) = 1 \qquad Equation 22$$

### Heat Transfer Model of the Supporting Structure in the Deploying Process

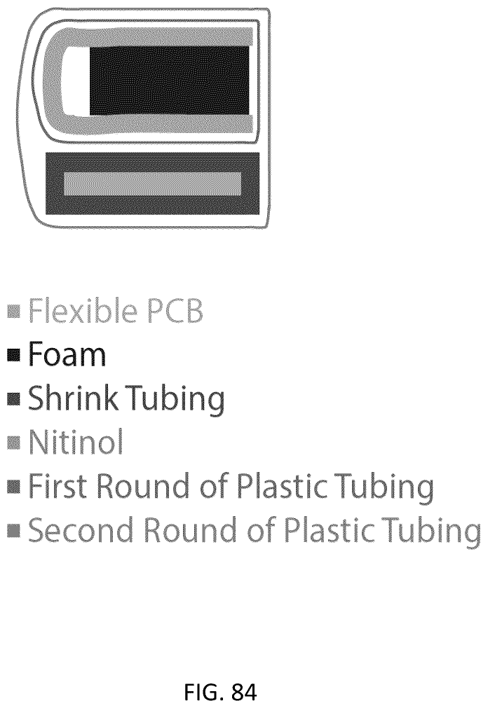

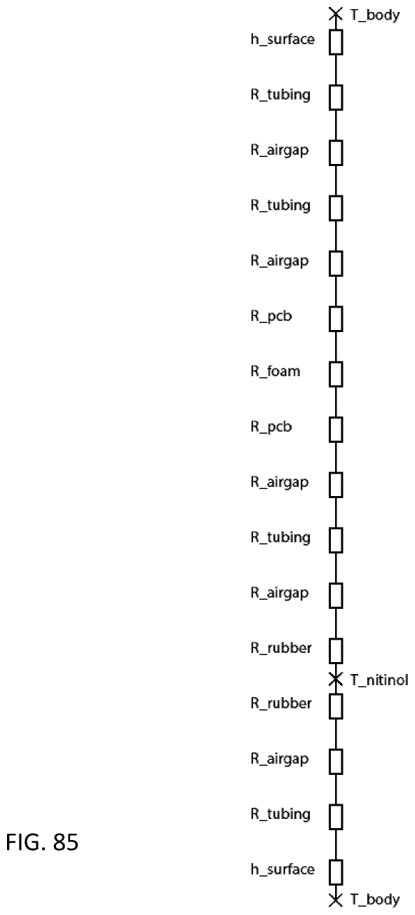

To understand the heat transfer process, a thermal resistance network was used to calculate the heat resistance in the sensing probe. A cross-section of the sensor probe is shown in FIG. 84 and its corresponding heat resistance network is shown in FIG. 85. On the sensor probe, pressure sensors of 1 cm width are equally spaced by 2 cm. On the side with pressure sensors, there are 1 cm wide air pockets sandwiched by the sensors. The heights of these air pockets are in the same order of magnitude with the sensor, so to simplify the calculation, the heat resistance of the air pocket was assumed to be the same as the sensor. The Biot number along the thickness of the strip is 0.005 while the Biot number along the length is 8.98. This means the internal heat resistance along the thickness of the nitinol strip is very small compared with the external heat resistance. On the other hand, the heat resistance along the length of the nitinol can be large compared with external heat resistance. The probe can reach a relatively uniform temperature distribution along the thickness but the temperature along the length can vary. This is because the heat resistance along the length can be an order of magnitude larger than the outer layer heat resistance. The strip may not be able to reach a relatively uniform temperature distribution along the length. With this insight, a heat transfer model of the strip was built with the following assumptions: 1) the temperature across the thickness is uniform; 2) the body of the swine is an infinite heat resource; 3) the strip is adiabatic along its length. Correspondingly, the governing equation of one piece of probe is described by Equation 20, where c is the heat capacity of nitinol in the cross section, T is the temperature of the nitinol, T_{body} is the internal temperature of the subject's GI tract, and R is the heat resistance of the nitinol:

$$C \frac{d T}{dt} = \frac{{T}_{b ody} - T}{R} \qquad Equation 23$$

The specific heat resistance Rin Km/W is calculated as follows, with R_{foamside} being the heat resistance on the side with foam and R_{foamwithout} being the heat resistance on the side without foam, which are connected in series:

$$R = \frac{1}{\frac{1}{{R}_{foamside}} + \frac{1}{{R}_{foam without}}} \qquad Equation 24$$

To simplify the equation the relative temperature Q can be used:

$$Ω = T - {T}_{body} \qquad Equation 25$$

Thus, the temperature of a piece of nitinol strip in after time t in the body is calculated as follows, where C is the heat capacity of nitinol in the cross section per unit length and Oo is the initial relative temperature with T_{body} being the room temperature:

$$Ω = {Ω}_{0} exp ( - \frac{t}{R C} ) \qquad Equation 26$$

### Friction Forces in the Overtube

To estimate the force required to insert and extract the coiled sensor belt through the over-tube, a simplified model is devised based on the helical nature of the sensor belt: the force required to extract or insert the sensor is the force required to overcome the static friction between the wall of the over-tube and the coiled sensor, which is obtained from the radial force exerted by the sensor coil to the wall of the over-tube, and a friction coefficient.

The whole coiled sensor belt, which has 10 turns, is modeled as 10 helical springs connected in series in which each spring represents one turn of the actual coil. Each modeled spring's diameter equals the major diameter of the corresponding turn of the relaxed sensor belt. The spring element has a rectangular cross section with thickness of 0.5 mm and width of 10 mm.

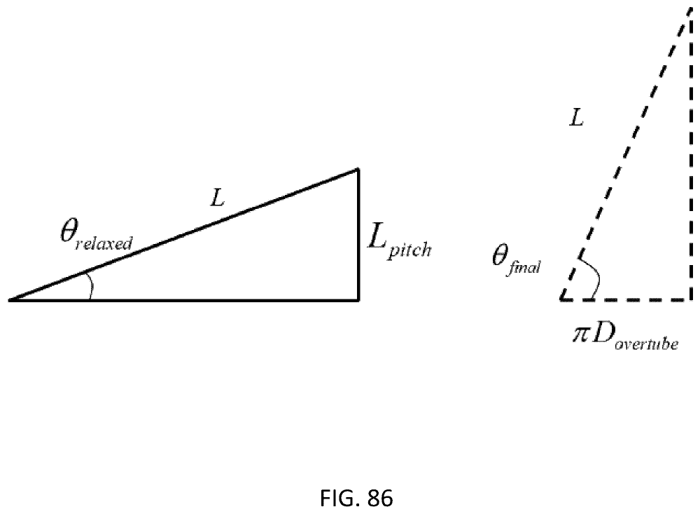

The torque required to impose a twist of angle θ can be expressed as in Equation 27. In which G is the torsion modulus, J is the area inertia of the sensor belt and L is the straightened length of the sensor-belt forming this turn. The angle of twist (see FIG. 86) is calculated from the change of pitch angle as shown in Equation 28, in which G is the torsion modulus, J is the area inertia of the sensor belt, and L is the straightened length of the sensor-belt forming this turn.

$$T = \frac{GJ θ}{L} \qquad Equation 27$$

$$θ = {θ}_{final} - {θ}_{relaxed} = {sin}^{- 1} ( \frac{{L}_{pitch}}{L} ) - {cos}^{- 1} ( \frac{π {D}_{overtube}}{L} ) \qquad Equation 28$$

In order to simplify the model, this model assumes that the tension or compression applied to the ends of the coiled sensor belt does not change the outer diameter of the coil; the force that initially deforms the sensor belt into the over-tube that is exerted at the ends of the over-tube, is ignored; and the axial twist of the whole modeled coil spring is assumed to be equivalent to the twist imposed on the spring element, since as the coil is deformed to be extracted through the over-tube, its pitch angle very closely approaches 90 degrees, i.e. the coil is almost straightened. Put another way, the model assumes that the force applied to a particular coil linearly distributes along the length of the turn.

As the coiled sensor belt is squeezed into the overtube, its diameter is forced to decrease from relaxed diameter to the diameter of the overtube. As the deformation of the spring is purely torsional per the assumptions above, the energy method is employed to relate the torque on the belt itself to the radial force applied by the over-tube onto the coil as shown in Equation 29:

$$T Δ θ = F Δ R \qquad Equation 29$$

When the coiled sensor belt is pulled into the over-tube, the coil's diameter must be smaller than the inside diameter of the over-tube, D. By modeling each turn of the spring as a loop and letting f be the force density (N/m) acting on the outer diameter, the relationship between the change in radius, dR, and the change in twist angle of each turn of the spring, dθ, can be expressed by Equation 30:

$$Td θ = f π Ddr \qquad Equation 30$$

Twisting of the coil spring decreases its diameter. Since the twist of the whole spring is assumed to be equivalent to the twist of the spring element (e.g. to be twisted uniformly), the relationship between dθ and dR can also be expressed as follows, with r₀ representing the radius of the loop prior to the imposition of the change in twist angle dθ and r₁ representing the radius of the loop after the imposition of the change in twist angle de:

$${r}_{1} ( 2 π + d θ ) = {r}_{0} 2 π \qquad Equation 31$$

$${r}_{1} \approx {r}_{0} ( 1 - \frac{d θ}{2 π} ) \qquad Equation 32$$

$$dr = {r}_{0} - {r}_{1} = {r}_{0} \frac{d θ}{2 π} \qquad Equation 33$$

By substituting Equation. 33 into Equation. 30, the relationship between the torque applied and the radial force per unit loop length can be expressed as in Equations 34 and 35 where we also use r=D/2, where D is the diameter of the turn.

$$T = \frac{{fD}^{2}}{4} \qquad Equation 34$$

$$f = \frac{4 T}{{D}^{2}} \qquad Equation 35$$

Finally, the friction between the wall of the over-tube and the coiled sensor for each coil can be expressed as in Equation 36 in which μ represents the friction coefficient between the inner wall of the over-tube and the coiled sensor belt, where the turn is confined into a tube with diameter D_{overtube}, and D is therefore equal to D_{overtube}:

$${F}_{f} = μ \frac{4 π T}{{D}_{ovetube}} \qquad Equation 36$$

In order to calculate the force required for sensor insertion and extraction as a function of the length of the sensor belt inside the stomach, a further assumption is made that the friction is constant within each turn of the coil. F_{f} is retro-fitted to f_{f}, the friction per unit length of the sensor belt, and linearly interpolated along the length of the sensor belt. f_{f} is then integrated for the length of the sensor belt within the over-tube to obtain the force required to overcome the static friction at each point of displacement and plotted as in FIGS. 21 and 22.

As the coiled sensor belt is squeezed into the over-tube, its diameter is forced to decrease from relaxed diameter to the diameter of the over-tube. As the deformation of the spring is purely torsional per the assumptions above, the energy method is employed to relate the torque on the belt itself to the radial force applied by the over-tube onto the coil as shown in Equation 37:

$$T Δ θ = F Δ R \qquad Equation 37$$

When the coiled sensor belt is pulled into the over-tube, the coil assumes the inside diameter of the over-tube, D. By modeling each turn of the spring as a loop and let f be the force density (N/m) acting on the outer diameter, the relationship between the change in radius, dR and change in twist angle, dθ of each turn of the spring can be expressed as in Equation 38:

$$T d θ = f π D d r \qquad Equation 38$$

Naturally, twisting the coiled spring decreases its diameter. Since the twist of the whole spring is assumed to be equivalent to the twist of the spring element, the relationship between dθ and dR can also be expressed as in Equations 39-41. In which r₀ represents the radius of the loop prior to the imposition of dθ and r₁ represents the radius of the loop after dθ have been imposed.

$${r}_{1} ( 2 π + d θ ) = {r}_{0} 2 π \qquad Equation 39$$

$${r}_{1} \approx {r}_{0} ( 1 - \frac{d θ}{2 π} ) \qquad Equation 40$$

$$dr = {r}_{0} - {r}_{1} = {r}_{0} \frac{d θ}{2 π} \qquad Equation 41$$

By substituting Equation 30 into Equation 27, the relationship between the torque applied and the radial force per unit loop length can be expressed as in Equations 42 and 43.

$$T = \frac{{fD}^{2}}{4} \qquad Equation 42$$

$$f = \frac{4 T}{{D}^{2}} \qquad Equation 43$$

Finally, the friction F_{f} between the wall of the over-tube and the coiled sensor for each coil can be expressed as in Equation 44 in which μ represents the friction coefficient between the inner wall of the over-tube and the coiled sensor belt.

$${F}_{f} = μ \frac{4 π T}{{D}_{overtube}} \qquad Equation 44$$

In order to calculate the force required for sensor insertion and extraction as a function of the length of the sensor belt inside the stomach, a further assumption is made that the friction is constant within each turn of the coil. F_{f} is retro-fitted to f_{f}, the friction per unit length of the sensor belt, and linearly interpolated along the length of the sensor belt. f_{f} is then integrated for the length of the sensor belt within the over-tube to obtain the force required to overcome the static friction at each point of displacement and plotted as in FIGS. 21 and 22.

### Scanning Board Design and Fabrication

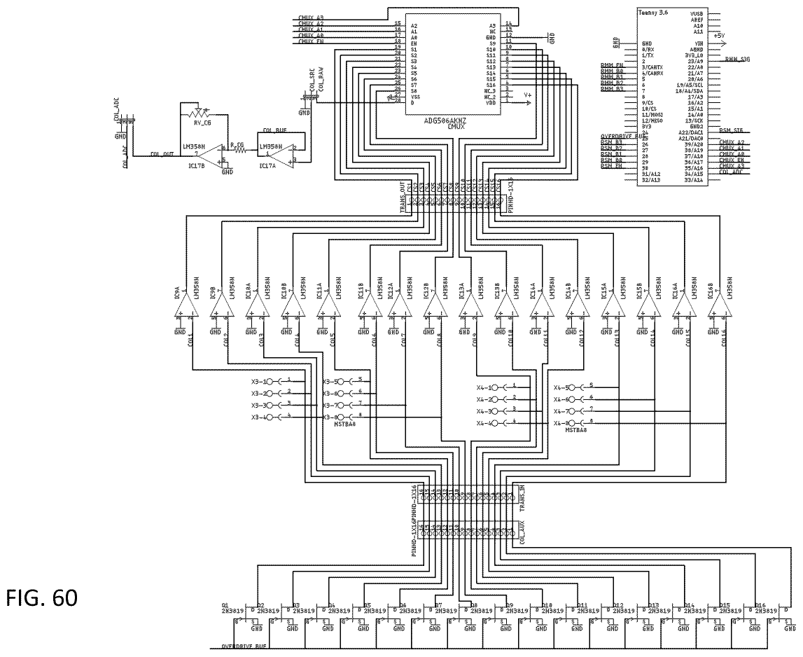

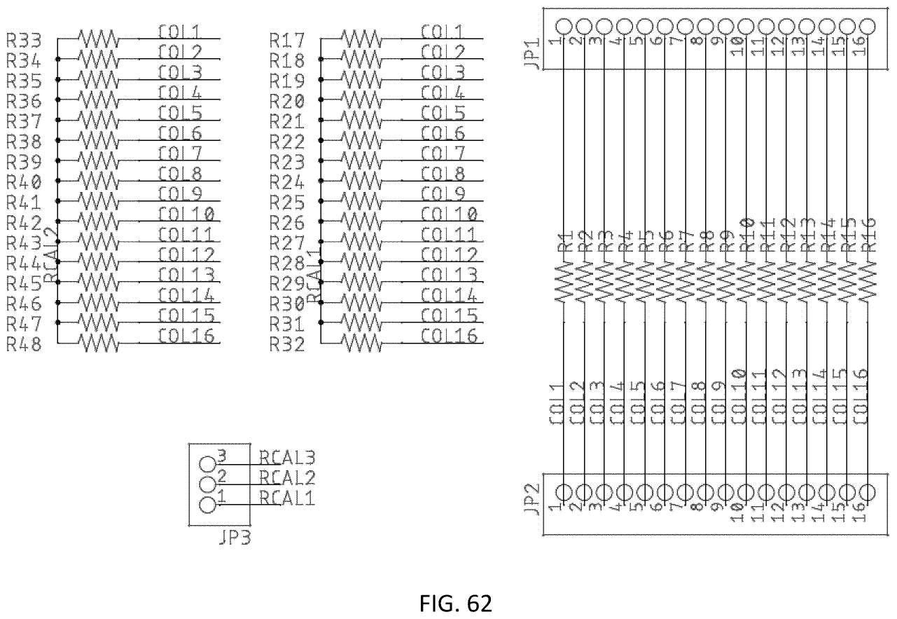

FIGS. 59 and 60 show the full circuit diagram of the motherboard of the pressure mapping system. The connector RMUX8 and RMUX16 are the sockets of CD74HC4060 multiplexers mounted on breakout boards (DigiKey P/N 1568-1181-ND). Similarly, RSMUX8 and RSMUX16 are the sockets of CD74HC4060 multiplexers mounted on breakout boards (DigiKey P/N 1568-1181-ND). Connector X1-2 holds a 16-pin Molex (DigiKey P/N 102-6418-ND) for row connections of the sensing matrix. Connector X3-4 holds a 16-pin Molex (DigiKey P/N 102-6418-ND) for column connections of the sensing matrix. FIG. 62 shows the circuit diagram of the daughterboard with all reference resistors and feedback resistors. As governed by Equation 1, the feedback resistors' must be optimized to produce a measurable voltage without saturating the amplifier or causing excessive current (FIG. 71). In this design, reference resistors are 1.00 KOhm (DigiKey P/N RNCF1206TKY1K00CT-ND) and 10.00 KOhm (DigiKey P/N 10KADCT-ND), and feedback resistors are 4.70 KOhm (DigiKey P/N 4.7KADCT-ND). Placing these resistors on a daughterboard enables the measurement of various ranges of resistances by replacing daughterboards with another.

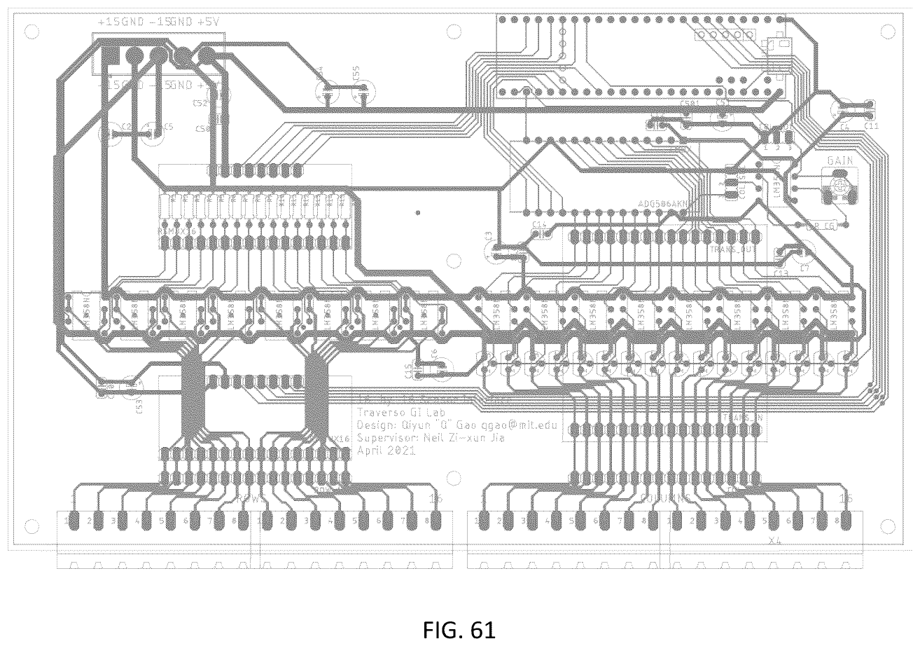

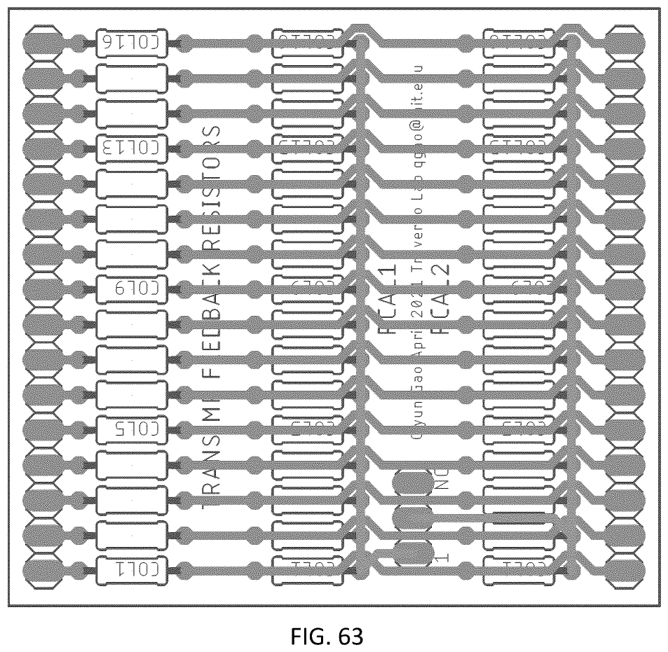

The connectors TRANS_IN and TRANS_OUT connect to the daughterboard. They connect the feedback resistors on the daughterboard to the output and negative feedback port of each operational amplifier (FIG. 60) (LM358N, DigiKey P/N 296-9554-5-ND). Since the connector TRANS_IN is connected to X3-4 (FIG. 60), reference resistor R17-R48 (FIG. 62) (DigiKey P/N 4.7KADCT-ND) shares the column connections in the sensing array. Therefore, these reference resistors become additional rows to the sensing array. The row connections of reference resistors are made through JP3 (FIG. 62) to connect ROW_AUX (FIG. 60) via jumper wires and become row 14 and 15. FIGS. 61 and 63 show the PCB layout of the motherboard and daughterboard. Boards are manufactured with FR-4 process, 1.6 mm board thickness and 1 oz copper density.

### Scanning Workflow

A simplified circuit layout is shown in FIG. 16. The microcontroller (Teensy 3.6, DigiKey P/N 1568-1442-ND) generates a driving voltage through its digital to analog converter (DAC) and selects one row to apply the driving voltage through the row scanning multiplexer (MX1) (DigiKey P/N 1568-1181-ND) while leaving other rows at ground potential (referred to as ‘selecting’ a row). The driving voltage or ground potential is then applied to each row by a bank of voltage buffers (RA0-15) (DigiKey P/N 296-9554-5-ND). Since column connections are connected to the negative port of transimpedance amplifiers (CA0-15) (DigiKey P/N 296-9554-5-ND), columns are kept at ground potential. Therefore, current only flows through sensing elements on the row with driving voltage applied. Other sensing elements have no current through them since they have ground potential on both sides. The current on each column is converted to voltage by transimpedance amplifiers according to Equation 1. As the current on each column is only through one sensing element, resistance or conductance of each sensing element can be measured individually without cross-talk.

The output voltage of the transimpedance amplifier on each column is then fed into the column selection multiplexer (MX3) (DigiKey P/N ADG506AKNZ-ND) that screens through all columns while one row is selected. The output of the column selection multiplexer is buffered by amplifier FA-A (DigiKey P/N 2156-NE5532P-ND) and then flipped to a positive voltage with an appropriate gain by inverting amplifier FA-B (DigiKey P/N 2156-NE5532P-ND), and finally sampled by an analog to digital converter (ADC). In the meantime, the row monitoring multiplexer (MX2) (DigiKey P/N 1568-1181-ND) connects the selected row to another ADC in order to accurately measure the voltage on the selected row. Therefore, with voltage across and current through the sensing element, its resistance can be accurately measured.

Furthermore, as the capacitance of the sensing element is non-trivial, the system needs a current input large enough to pull row connections to the driving voltage or back to ground potential. Row currents are directly provided by voltage buffers, but column current is limited by feedback resistors. To alleviate this, bypass transistors (Q1-Q15) (DigiKey P/N 2N7000BU-ND) are connected from column connections directly to ground in order to provide a low-resistance path to quickly charge and discharge the capacitance of sensing elements, thereby increasing the row switching speed and the frame rate of the system. The automated protocol of measuring the resistance of the whole sensing matrix is as follows: (1) turn on Q0-Q15; (2) set MX1 to the apply driving voltage to the desired row; (3) set MX2 to the row driving voltage being applied; (4) turn off Q0-Q15; (5) cycle MX3 through channel 0-15 and sample its output voltage; and (6) repeat steps 1-5 until all rows are measured.

Self-Calibration and Interface

As shown in FIG. 16, output voltage of each column is fed into the column multiplexer, followed by a buffer and an inverting amplifier with a gain of K. Therefore, voltage fed into ADC can be expressed as Equation 45, or simplified to Equation 45, in which K is a known constant. If driving voltage, feedback resistance, and gain of the inverting amplifier are held constants, then c, the zero-point drift of op-amp, is also a constant. However, the feedback resistance and gains are subject to manufacturing tolerance and thermal drift, so two reference rows consisting of precision reference resistors are included in order to calibrate with two known resistances, R₁ and R₂, on each column. In this design, R₁ and R₂ are 1.00 kOhm (DigiKey P/N RNCF1206TKY1K00CT-ND) and 10.00 kOhm (DigiKey P/N 10KADCT-ND) respectively. The reference resistors are sharing column connections with the array of sensing elements. In this implementation, R₁ and R₂ are optimized to have conductance G₁, which produces a near full-scale ADC reading, and G₂, which is the lower-bound of the resistance measured. Therefore, the variation in K between columns can be compensated since reference resistors are subject to the same K as the foam resistances being measured.

In a measurement sequence, V₁ and V₂ are the measured ADC readings when the rows consisting of R₁ and R₂ are not grounded. The V_{foam}'s, which are the ADC readings when the rows consisting of foam sensing elements are active, are also measured sequentially. In each column, the sensed voltages of each resistor are subject to the same k (Equations 47-48). Since G₁ and G₂ are known at this step, G_{foam} can be calculated without knowing K. This scanning structure makes the measured results less sensitive to variations in the feedback resistors, thermal drift, and fluctuations in the amplifiers. The factor of k lumps all the parameters together in the measurement. And taking the advantage of the self-calibration, we are able to eliminate the k in the equations and obtain Equation 50 from Equations 45-49.

$${V}_{ADC} = {G}_{foam} {V}_{d rive} {R}_{feedback} K + c \qquad Equation 45$$

$${V}_{A D C} = {G}_{foam} k + c \qquad Equation 46$$

$${V}_{1} = {G}_{1} k + c \qquad Equation 47$$

$${V}_{2} = {G}_{2} k + c \qquad Equation 48$$

$${V}_{foam} = {G}_{foam} k + c \qquad Equation 49$$

$${G}_{foam} = \frac{{V}_{foam} - {V}_{2}}{( {V}_{1} - {V}_{2} ) ( {G}_{1} - {G}_{2} ) + {G}_{2}} \qquad Equation 50$$

### Testing Protocols of the Scanning Board

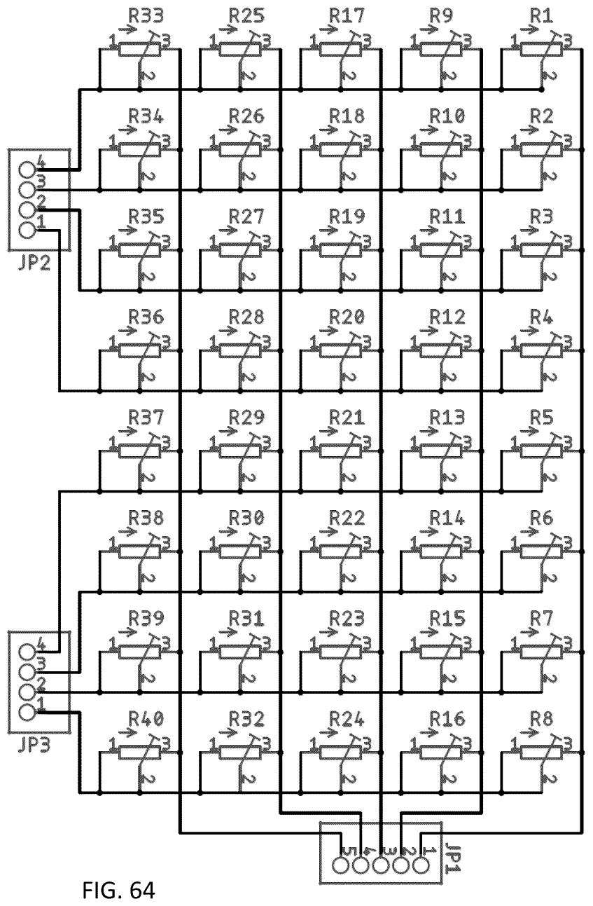

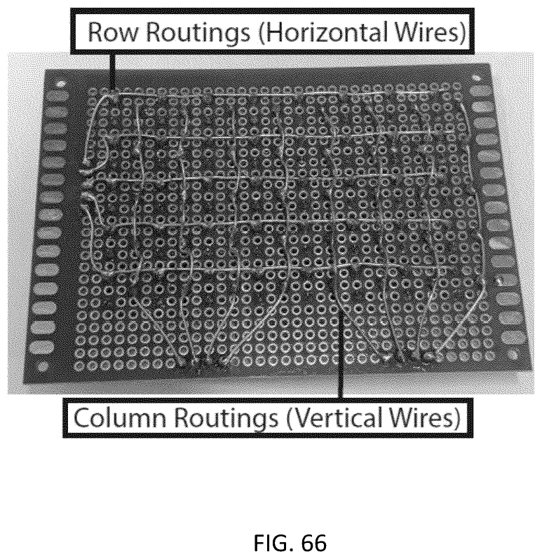

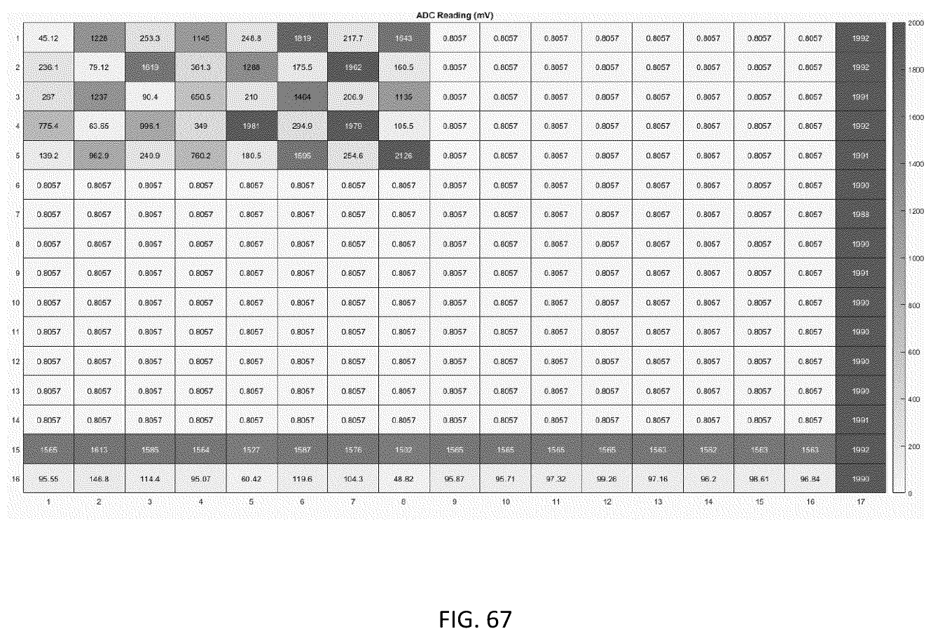

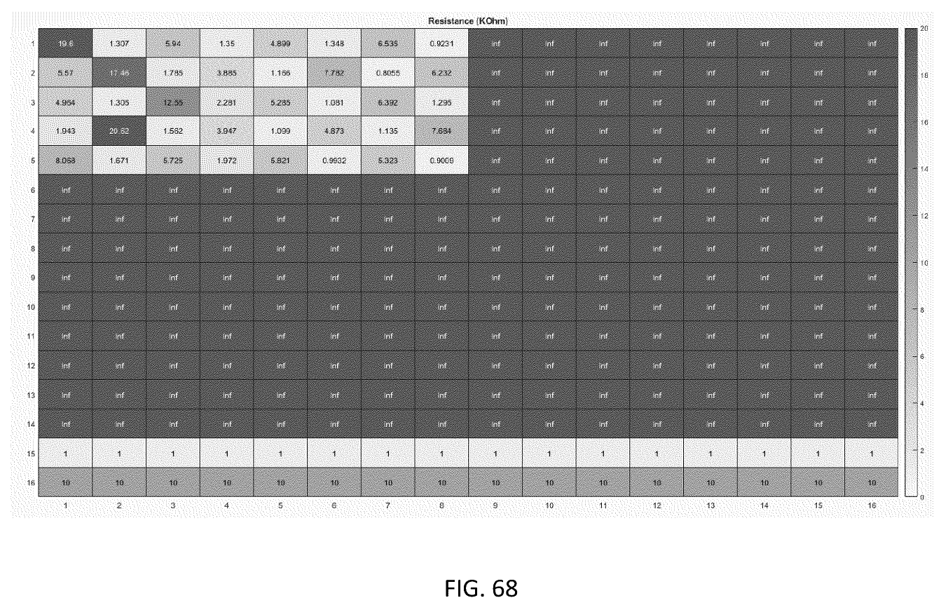

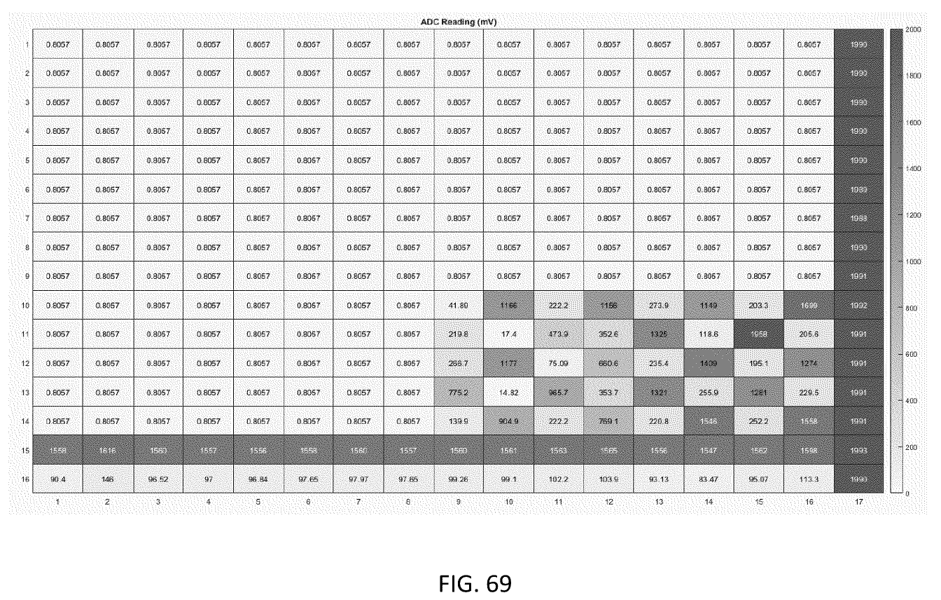

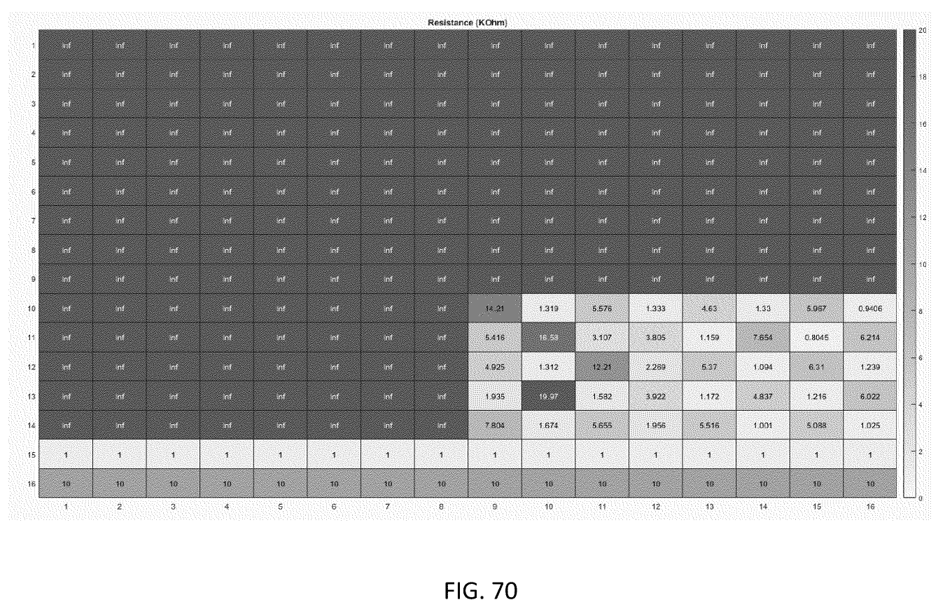

The pressure mapping system is tested by a variable resistor array and a physical test pattern on sensing elements. The circuit diagram of the variable resistor array is shown in FIG. 64, in which JP1 makes 5 row connections and JP2-3 makes 8 column connections. FIGS. 65 and 66 show the physical variable resistor array consisting of 40 trimmer potentiometers (Amazon P/N B07J4NR372). The results are shown in FIGS. 67-70. The variable resistor array is connected to row 0-4, column 0-7 for FIGS. 67 and 68, and to row 9-13, column 8-15 for FIGS. 69 and 70, with FIGS. 67 and 69 showing the voltages measured by ADC and FIGS. 68 and 70 showing the measured resistance. The variable resistor array's value is set into a checkerboard pattern with a range of resistances to reveal any cross-talks and signal degradations. The resistances were also measured by handheld multimeter (Innova 3220). The resistance measured by the pressure mapping system coincides with multimeter reading for 3 significant figures, indicating an error less than 1%.

The performance of the system was evaluated by a variable-resistor array and by a physical test pattern pressed against the sensing elements (FIGS. 65-70). FIG. 71 was used to calculate the value of the sensing element resistance from the voltage readings. A long belt of sensors was placed into a serpent-like shape under a set pattern to fit into the testing platform (FIG. 72). The physical test pattern is shown in FIG. 73 with protrusions forming the letters ‘MIT’ when stacked. The width of each protrusion is 20 mm, matching the spacing of sensing elements, and the thickness of the plate is ¼ inch (6.35 mm). A belt-shaped sensor with sensing elements arranged linearly is used in this test. The plates are held on an Instron and pressed into the belt-shaped sensing matrix with the protrusions aligned with sensing elements. The pattern is driven into the sensor with a force of 5N. The result is shown as a heatmap in FIG. 74 . . . . Such a test pattern causes alternating high-pressure and low-pressure points on the sensor array, demonstrating the MIT letters clearly while also showing the absence of cross-talk between sensing elements as observed in both evaluations (FIG. 74). This result shows the system can map complex pressure pattern thus paves way for further testing.

Fabrication of the Bolus Supporting Structure and the Integration with the Pressure Sensor

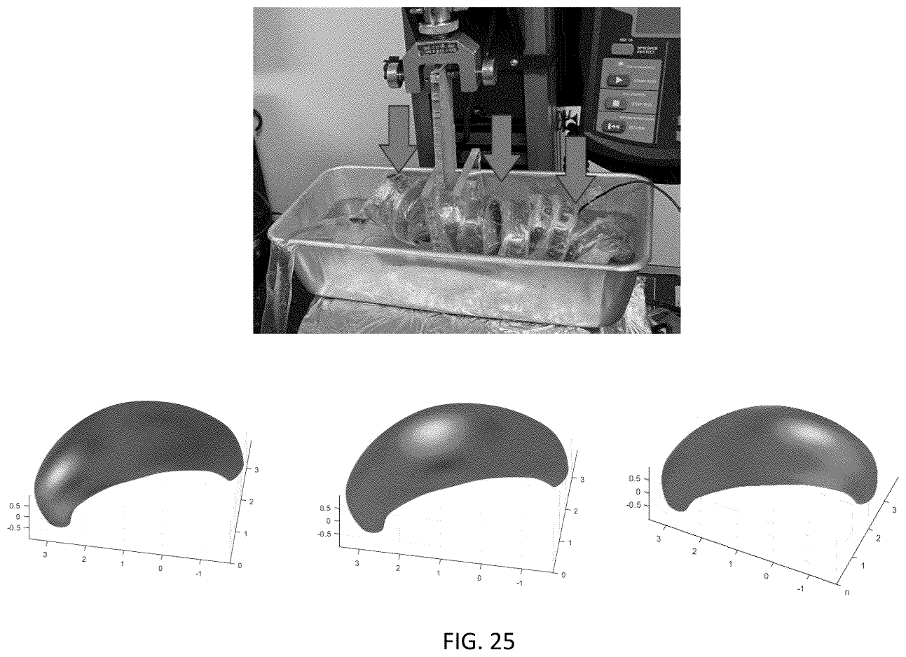

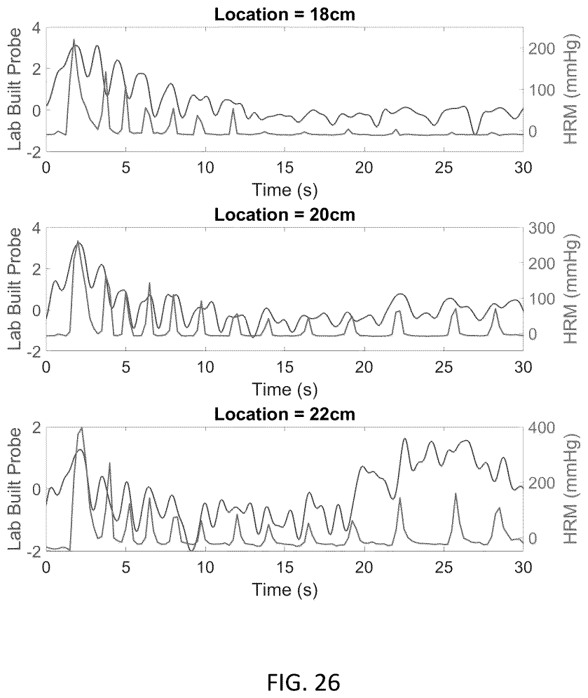

The fabrication process of the bolus supporting structure (e.g., the globular supporting structure) is shown in FIG. 24. A Nitinol sheet of 1 m long, 50 cm wide, and 0.5 mm thick was purchased from Kellogg's Research Labs. A water jetting bed (OMAX) was used to cut two strips of 95 cm long, 1 cm wide from the sheet. The two strips were then riveted together. A CAD model of the fixture was made on Fusion 360, and the manufacturing G code was exported from it. A Tormach 440 CNC mill was used to mill the fixture out of a rod of plastic. The plastic fixture was used for sand casting the bronze fixture. The bronze fixture was then drilled and tapped with thread holes. The riveted strip was fixed onto the bronze fixture via bolts. Then the entire structure was placed in the furnace at 600 C for 1 hour to program the shape of the Nitinol. The supporting structure was then inserted into ½ inch shrink tubing (WindyNation 100 Feet ½ inch). A heat gun was used to heat the tubing and make a tight cover on the nitinol supporting structure. This is to prevent the edges from tearing the sealing layer. Double sided conductive tapes (3M Feb. 5, 9719) were applied on both sides of the 16-1231 packaging foam sheet. A puncher with diameter ¼ inch was used to punch foam cylinders from the double sided packaging foam. The foam cylinders were then placed on the FPC sensor band after peeling off the cover of the double sided tapes. The assembled sensor band was placed inside a 1 inch diameter Low Density Polyethylene (LDPE) tubing (Uline S-3520). The tubing was then adhered to the supporting structure using non-conductive double sided tapes. Another layer of LDPE was then placed over the entire structure. 40 g of well mixed Ecoflex 00-20 silicone mixture was injected into the space between two layers of LDPE tubing. The probe was left in the lab overnight under room temperature to have the silicone cure. The finished probe was then placed inside a 40 C water bath. An Instron was used to press the head, body, and the tail of the probe. The corresponding results were shown in FIG. 25.

Ex vivo Calibration of Tubular System

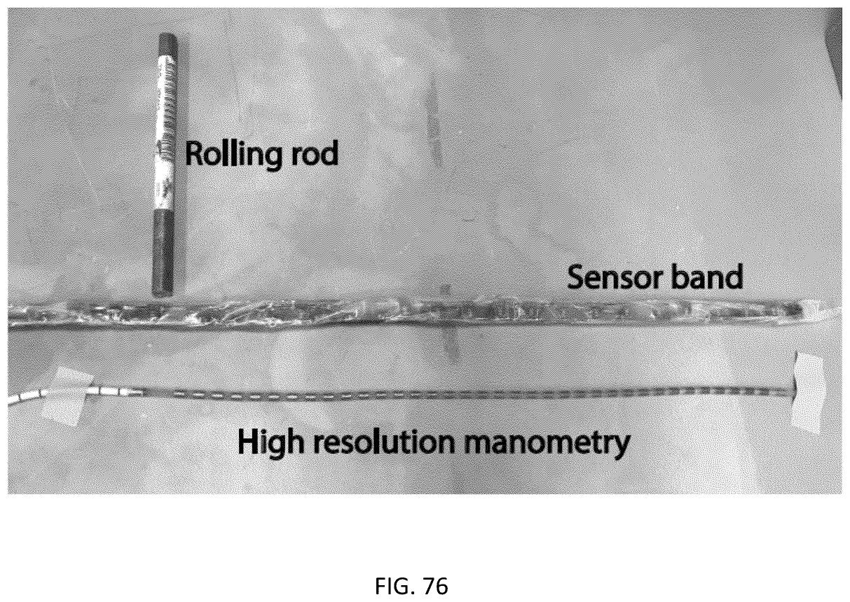

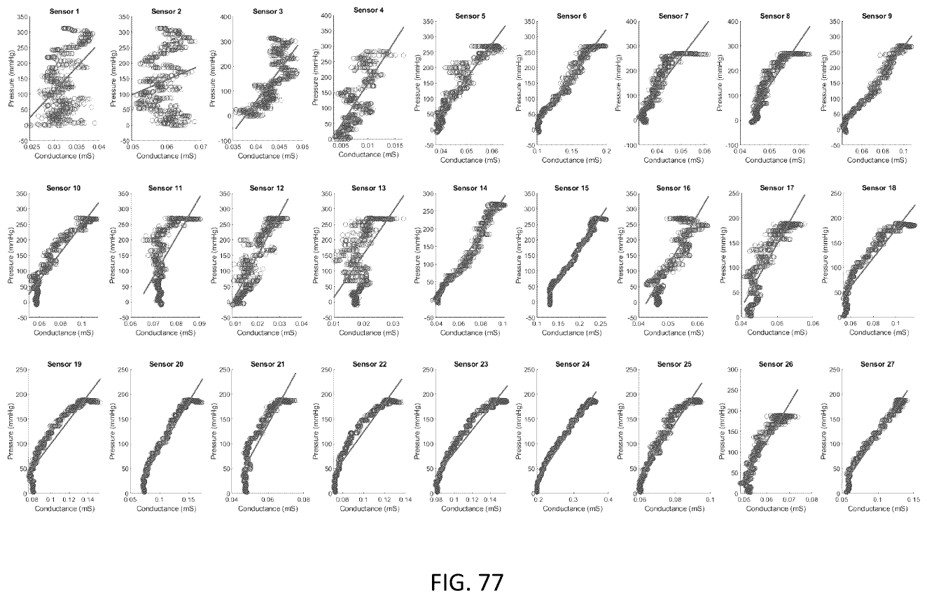

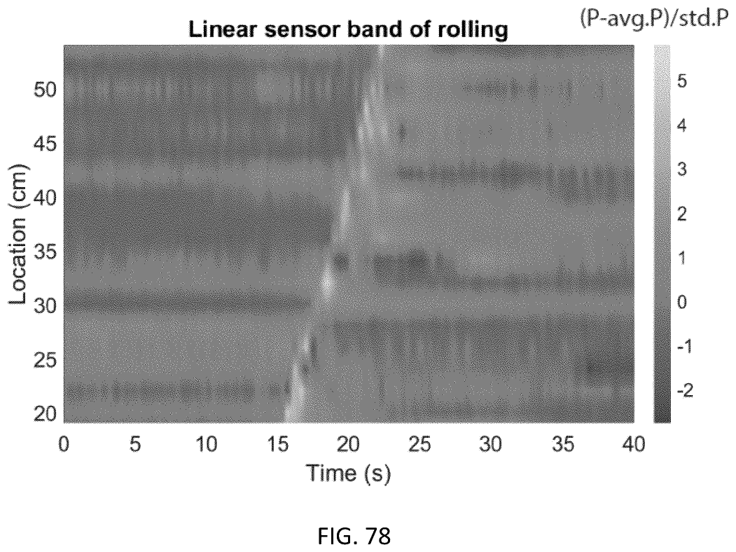

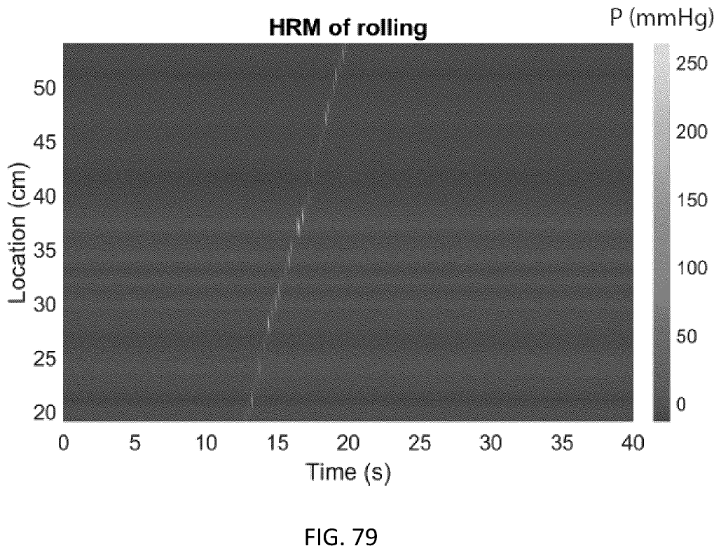

The benchtop setup of the ex vivo calibration is shown in FIG. 25. The tubular organ (e.g., linear) sensing probe and a probe of HRM were sandwiched between a pneumatic bladder and a wood board covered with foam. The pneumatic bladder was then inflated/deflated. Data obtained from the sensor band and HRM probe are synchronized and plotted in FIG. 77. The x-axis is the recorded conductance from each pressure sensor in the sensor band, and the y-axis is the pressure reading from the HRM probe. Except for two pressure sensors, the conductance and the pressure readings have an approximately linear correlation. This data is then used as calibration for future studies. To further test the time resolution of the tubular sensing probe, the tubular sensing probe was taped to the bench along with the probe of HRM. A rolling rod was then rolled over the probe and sensor band at the same time, shown in FIG. 76. The data obtained from the rolling test is plotted in FIGS. 78 and 79. The x-axis is the time, y-axis is the location of pressure sensors on the sensor band/probe, and the color shows the value of pressure. The rolling test data from sensor band and probe show great similarity.

### Ex Vivo Calibration of Bolus System

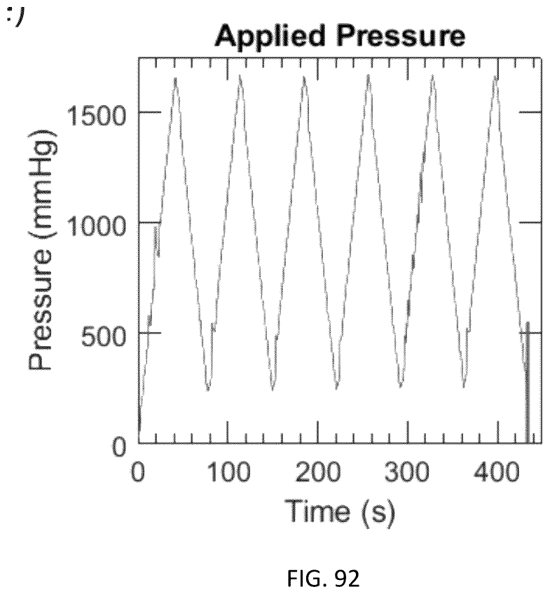

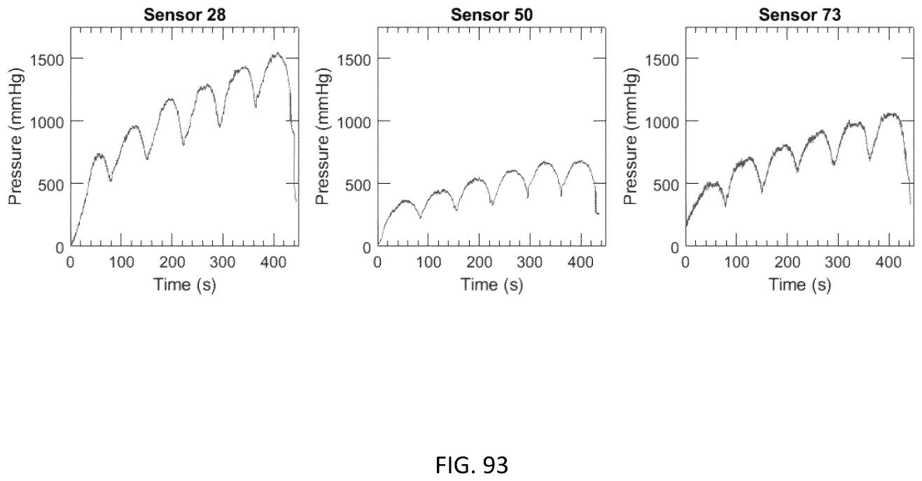

The bolus sensing probe's ability on location mapping and pressure response was validated. Then individual sensor foam pieces were calibrated in groups to compensate for variation among pieces. Localization and pressure response validation is performed as shown in FIG. 25. The sensor belt was first immersed in a water bath heated to 40 degrees Celsius to turn the sensing probe into the programmed shape. Sensor numbers 28, 50 and 73, indicated by arrows in FIG. 25 in left-to-right order, were tested. The sensing probe was mounted on a structure that provided support. Each sensor was subject to a pressure cycle shown in FIG. 92. The output of each of sensors 28, 50 and 73 is shown in FIG. 93.

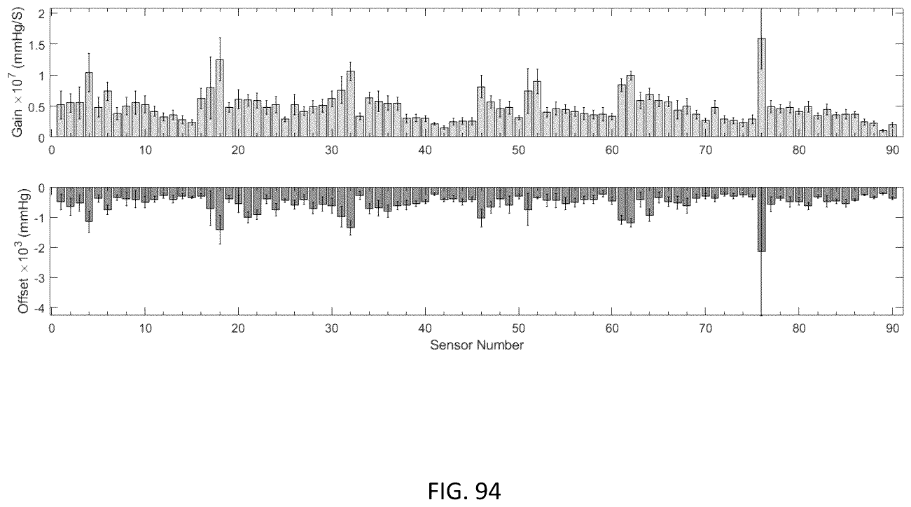

Sensor calibration was performed on an Instron platform. A flat pressure plate fixed on the Instron grasp jogged down onto the sensor belt mounted flat on a ¼ inch aluminum plate. The pressure plate was large enough to cover six pressure sensors. For each run, a ramping force with a peak amplitude of 5N was evenly distributed among the 6 sensors being calibrated. The applied force ramp consisted of 6 cycles, and the duration of each calibration was 300 seconds. The force applied to and resultant conductance of each sensor were linearly fitted using “cftools” in the MATLAB environment. The ‘gains’ (slopes of the fitted lines, representing the sensitivity of the sensor) and ‘offsets’ were subtracted for each sensor. The gain and offset of each sensor is shown in FIG. 94 with error bars representing the 95% confidence interval.

### Radial Stiffness Measuring

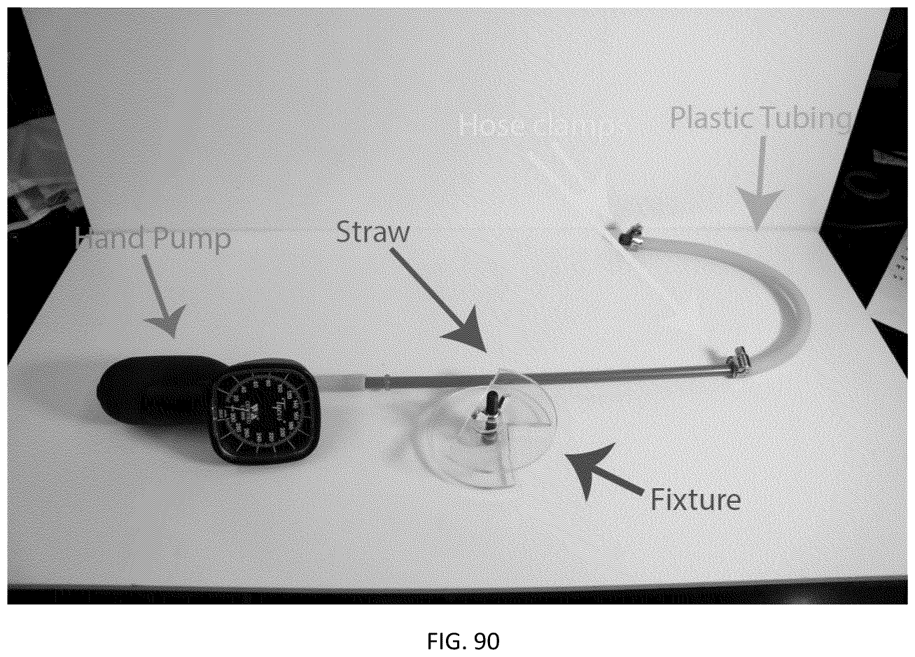

A series of experiments was conducted to measure the radial stiffness of the bolus supporting structure, and the equipment is shown in FIG. 90. The measuring device was composed of a hand pump (orange arrow), a rigid plastic straw (pink arrow), and plastic tubing (green arrow) whose ends were secured with hose clamps (yellow arrows). A laser cut a fixture (red arrow) was used to ensure only one turn was being measured during each trial. The plastic tubing was used to record the change of pressure.

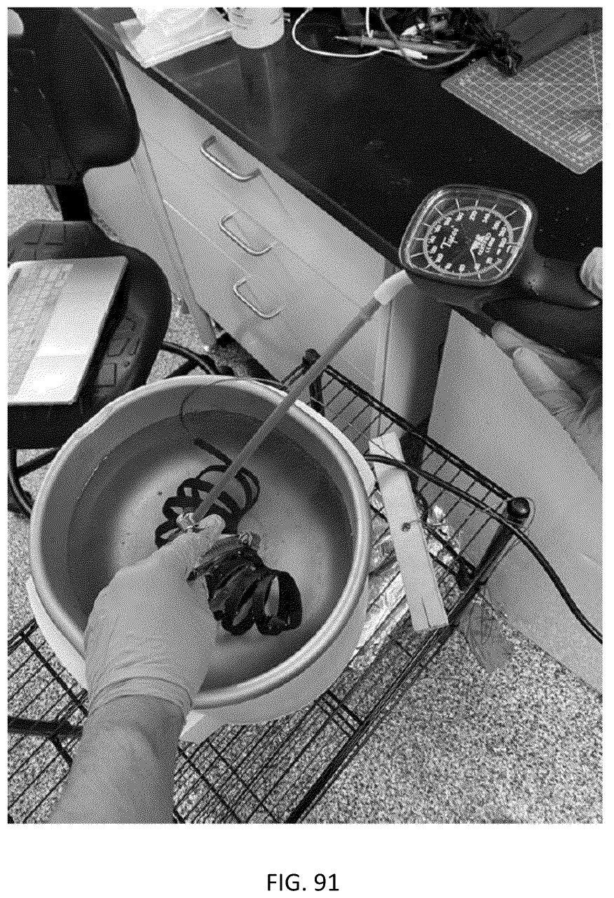

The setup is shown in FIG. 91. First, the coil was placed into a 40 degree Celsius water bath. The relationship between pressure and diameter was measured on each individual turn. Each turn was stabilized between the disks of the laser cut structure by adding a ring inside. The measuring device was wrapped around the turn and secured a hose clamp around it. The plastic tubing was inserted between the hose clamp and the coil. In each trial, the starting diameter was measured at a low pressure (See FIG. 20). The hose clamp screw was then tightened to decrease the diameter. The coil was forced to have a smaller diameter because of the pressure applied by the hose clamp. The air pressure and diameter were measured at regular intervals until the maximum pressure was reached. Then, the air was released by pressing the releasing button on the hand pump. This process was repeated three times.

### Deployment and Retrieval

The bolus probe was deployed and retrieved through the esophagus. During deployment (see FIG. 31), an overtube was placed in the pig's mouth that leads to the stomach. The overtube was lubricated with cooking oil spray. Then, the probe was then unwound and pushed through the overtube. A wire attached to the coil at the end (shown in blue). The coil was pushed through the esophagus (see FIG. 31) until the coil was fully in the stomach, which was confirmed with an endoscope. Once the nitinol was in the stomach, it coiled back into its programmed shape due to the temperature in the body (see FIG. 31). When the coil was ready to be removed, the string attached to it was pulled until all the equipment was fully out (see FIG. 31).

### In Vivo Evaluation Protocols on Swine Model

To validate the proposed concept, three Yorkshire swine, each weighing 75-100 kg, were used to test the platform. During the experiments, all three probes were deployed into the swine, and an electronic connection cable transmitted signals from the individual sensor units to the scanning interface. Data was then streamed to a PC as shown in FIG. 32. An electronic interface is developed to record the data streamed from in vivo probes.

A clinical HRM was used as the tool of calibration and comparison. The swine were anesthetized before the experiment and several stimulation methods were used to generate motility/reflex. The in vivo data collected in this study is processed in MATLAB using moving average filter to remove the noise came from heart beating in the animals. During in vivo experiments, we first deployed the sensing probes and HRM probes into the animals. We turned on the devices and observed the live-streamed baseline readings of HRM and sensing probes for 1-2 minutes. After confirming there were no motility/peristalsis/reflex patterns observed on both devices we then subsequently started applying stimulations to the animals.

Animal Sedation: Pigs were sedated with intramuscular injection of Midazolam 0.25 mg/kg with Dexdomitor 0.03 mg/kg and after intubation, anesthesia was maintained with isoflurane (1-3% in oxygen).

Recovery: Pigs are returned to their pen and sedation reversed intramuscularly with the reversal agent Atipamazole. If intubated, the pig is closely monitored until extubation and then is followed by monitoring of the recovery process until the pig is standing and considered bright, alert, and responsive (BAR).

Monitoring/Vitals throughout the study: After the pig is sedated and weighted, it is placed on a heated operating table with the additional thermal support of a heated blanket Opthalmic ointment is applied to both eyes. Once the pig is placed on isoflurane (1-2%) and oxygen (1-3%) either via a face mask or an endotracheal (ET) tube, it is then connected to an anesthesia monitoring machine in order to monitor vital signs every 15 minutes until returned to the pen.

Endoscopic Delivery: Device delivered to stomach via colonoscope placed in orgogastric tube/overtube. PENTAX EC-3870TLK (160 cm) for delivering drugs/devices to the stomach of larger pigs (>50 kg) and for retro flexing to visualize the entirety of the stomach.

Overtube specifications: Material: Urethane PVC; Inner diameter: ⅝ inches; Outer diameter: ¾ inches; Wall thickness: 1/16 inches.

### (1) Stomach

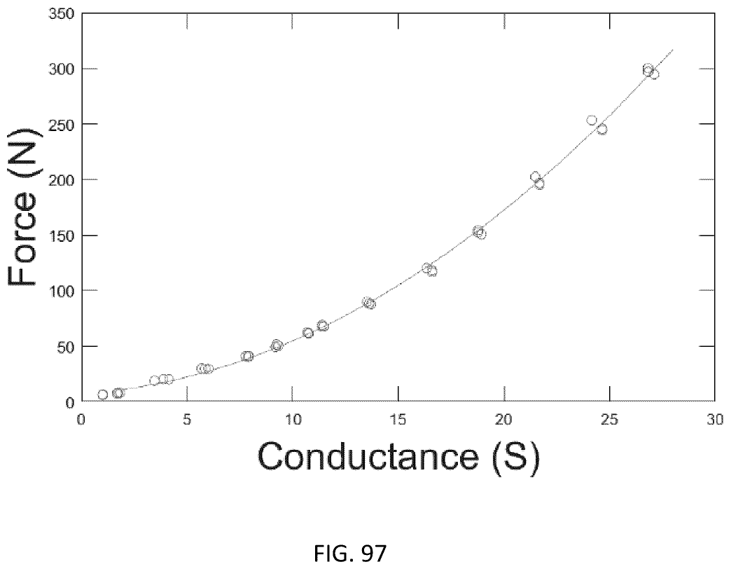

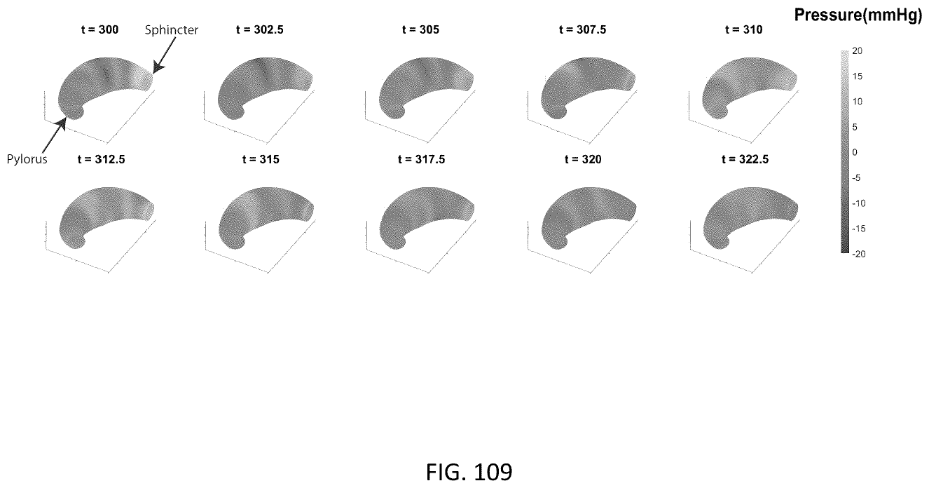

Once the nitinol coil is in the stomach, three tests were conducted: internal force, external force, and Azithromycin (USP) infusion. For internal force, a hose clamp was secured on the end of the overtube so that the stomach was a closed system. Using the endoscope, the stomach was inflated for 30 seconds then deflated for 30 seconds. External force was applied by applying palpation on the abdomen. On the abdomen was a pressure sensor, whose calibration is depicted in FIG. 97. The calibration graph was obtained with an Instron measuring force and multimeter measuring resistance simultaneously. Tests for external force were conducted for 10 minutes in cycles, where each cycle consisted of five seconds of applying and ten seconds of rest. While the sensing probe was in the animal's stomach, 500 mg of Azithromycin was infused intravenously over 30 mins using a syringe pump. The average pressure reading from the stomach sensing probe is shown in FIG. 98. We observed an increase in pressure during the first 20 mins of infusion followed by a period of relaxation. Measurements from all 90 sensors are shown in FIG. 99, as raw data, and FIG. 100, after applying a moving average filter. In FIG. 99, a very distinct respiration pattern was observed. In FIGS. 101 and 102, the height plot of the data from FIG. 100 and the 3D interface of the pressure distribution show a migration pattern. However, when comparing the peristalsis patterns in the FIGS. 101 and 102, we did not find any significant differences between the beginning of the infusion versus 25 min afterward. The pressure distribution inside stomach is shown in FIG. 109. Therefore, it does not appear the migration pattern is caused by Azithromycin.

### (2) Esophagus

A deflated bolus balloon was inserted into the pharynx along with the sensor probe and the HRM sensor catheter together. The tips of the two sensor probes were 20-30 cm behind the balloon. The balloon was inflated with 10 mL water and moved up and down the esophagus. This movement created a swallow reflex, and the data was recorded on both machines before repeating this process twice more with a five minute break in between. Once finished with the bolus balloon, tests were run with a dilation catheter. The sensor band, HRM sensor, and dilation catheter were inserted together roughly 20-30 cm into the esophagus. Using a syringe, the volume of the dilation catheter was increased to 10 mL and then decreased back to 0 mL. Each of these cycles took 30-60 seconds. Like the bolus balloon, three cycles were conducted with a five minute wait between each cycle.

### (3) Rectum

A bolus balloon was inserted into the rectum along with the HRM probe and the sensor band. The two sensing probes were 10 cm into the rectum. Data was recorded for one minute to obtain a baseline on both devices. At 80 s, the bolus balloon was inserted into the rectum. The balloon was then inflated with 20 mL water at 140 s and held for two minutes. The balloon was then deflated and held for two minutes. This process was repeated two more times.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present invention has been described in terms of one or more preferred embodiments, and it should be appreciated that many equivalents, alternatives, variations, and modifications, aside from those expressly stated, are possible and within the scope of the invention.

1. A catheter system for obtaining a pressure profile of a gastrointestinal (GI) tract, the catheter system comprising:
a catheter configured to be inserted endoluminally into the GI tract, the catheter including an outer sheath defining a first lumen and sensing unit enclosed within the first lumen, the sensing unit including:
a support structure made of a shape memory material configured to transition from a first configuration to a second configuration, wherein the second configuration includes a first region shaped to conform to a first portion of the GI tract and a second region shaped to conform to a second portion of the GI tract that is distinctly shaped from the first portion of the GI tract;
a flexible printed circuit board supported on the support structure; and
a plurality of pressure sensors spaced apart along the flexible printed circuit board,
wherein, following insertion of the catheter into the GI tract, the support structure causes the sensing unit to transition from the first configuration to the second configuration to position the plurality of pressure sensors to acquire pressure readings in three dimensions from the first portion of the GI tract and the second portion of the GI tract.

2. The catheter system of claim 1, further comprising a processor configured to receive the pressure readings in three dimensions from the plurality of pressure sensors and generate a 3D pressure mapping of the GI tract, including the first portion of the GI tract and the second portion of the GI tract.

3. The catheter system of claim 1, wherein the first configuration is a linear configuration and the second configuration is non-linear configuration such that at least one of the first region and the second region has a coiled shape.

4. The catheter system of claim 3, wherein the coiled shape is configured to position the plurality of pressure sensors along an exterior periphery of the coil to engage with walls of the GI tract.

5. The catheter system of claim 1, wherein the flexible printed circuit board and the plurality of pressure sensors are enclosed within a first inner sheath and the support structure is enclosed within a second inner sheath, which are removable from the first lumen of the outer sheath.

6. The catheter system of claim 1, wherein the plurality of pressure sensors are formed by the flexible printed circuit board.

7. The catheter system of claim 6, wherein each pressure sensor of the plurality of pressure sensors is formed by a u-shaped section of the flexible printed circuit board having a first arm and a second arm that are movable relative to one another.

8. The catheter system of claim 7, wherein each pressure sensor of the plurality of pressure sensors further includes a compression member that is disposed between the first arm and the second arm, such that relative movement of the first arm and the second arm compresses the compression member therebetween to change in an electrical property of the compression member that is indicative of a pressure being applied to the catheter.

9. The catheter system of claim 7, wherein the first arms of the plurality of pressure sensors are connected with one another to define a main body of the printed circuit board, which is configured to be supported on the support structure.

10. The catheter system of claim 1, wherein the support structure is configured to transform from the first configuration to the second configuration based on a change in temperature a support structure.

11. The catheter system of claim 10, wherein the compression member is an electrically conductive foam material.

12. A pressure sensor, comprising:
a flexible printed circuit board having at least one u-shaped section defining a first arm and a second arm; and
a compression member disposed between the first arm and the second arm so that a first end of the compression member is coupled to the first arm and a second end of the compression member is coupled to the second arm, the compression member being configured to receive a compression of the first and second arm to induce a change in an electrical resistance of the compression member correlated to a measure of the compression.

13. The pressure sensor of claim 12, wherein the compression member is an electrically conductive foam material.

14. The pressure sensor of claim 12, wherein the flexible printed circuit board is supported by a support structure made of a shape memory alloy configured to transition from a first configuration at a first temperature condition to a second configuration at a second temperature condition.

15. The pressure sensor of claim 12, further comprising a sheath that is configured to cover the flexible printed circuit board and the compression member.

16. A method of manufacturing a catheter system for obtaining a pressure profile of a gastrointestinal (GI) tract, method comprising:
securing a first end of a compression member to a first arm of a flexible printed circuit board;
folding a second arm of the flexible printed circuit board relative to the first arm so that first and second arms form a u-shape; and
securing a second end of the compression member to the second arm so that the compression member extends between the first and second arms, the compression member configured to be resiliently compressed by the first and second arms to induce a change in an electrical resistance of the compression member.

17. The method of claim 16, further comprising securing the flexible printed circuit board to a support structure made of a shape memory material configured to transition from a first configuration to a second configuration, wherein the second configuration includes a first region shaped to conform to a first portion of the GI tract and a second region shaped to conform to a second portion of the GI tract that is distinctly shaped from the first portion of the GI tract.

18. The method of claim 17, wherein at least one of the first region and the second region has a coiled shape when the support structure is in the second configuration.

19. The method of claim 18, wherein the coiled shape is configured to position the flexible printed circuit board along an exterior periphery of the coil and to position the support structure along an interior periphery of the coil.

20. The method of claim 17, further comprising:
inserting the flexible printed circuit board and the compression member into a first inner sheath;
inserting the support structure into a second inner sheath; and
inserting the first inner sheath and the second inner sheath into an outer sheath.
